(19) United States
(12) Patent Application Publication
(10) Pub. No.: US 2007/0264257 A1
(43) Pub. Date: Nov. 15, 2007

(54) Immunotherapy of autoimmune disorders

(76) Inventors: Kyriaki Dunussi-Joannopoulos, Belmont, MA (US); Anand P. Iyer, Randolph, NJ (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/246,541

(22) Filed: Oct. 11, 2005

Related U.S. Application Data
(60) Provisional application No. 60/616,647, filed on Oct. 8, 2004.
(60) Provisional application No. 60/686,001, filed on Jun. 1, 2005.

(51) Int. Cl. A61K 39/395; A61K 45/00; A61P 37/00

(52) U.S. Cl. 424/132.1; 424/130.1; 424/133.1; 424/135.1; 424/142.1; 424/144.1; 424/278.1; 514/789

(57) ABSTRACT

Compositions and methods for treating autoimmune diseases are described. In particular, the use of B cell depleting agents and cytotoxic drug/B cell depleting agent conjugates with a drug loading significantly higher than in previously reported procedures and with decreased aggregation and low conjugate fraction (LCF) in treating autoimmune diseases is described. Combination therapies and compositions for treating autoimmune diseases, including the B cell depleting agents, conjugates and/or anti-cytokine agents, are also described.

### FIELD OF THE INVENTION

The present invention relates to compounds, conjugates of compounds, compositions and combination therapies for treating autoimmune diseases, such as rheumatoid arthritis (RA), systemic lupus (SLE), immune cytopenias (e.g., idiopathic thrombocytopenic purpura and autoimmune hemolytic anemia), autoimmune vasculitis and/or associated conditions. In particular, the present invention relates to B cell depleting agents such as B cell surface antigen targeting antibodies having specificity for cell surface antigenic determinants and conjugates of such B cell depleting agents conjugated to a cytotoxic drug. For example, the invention relates to cytotoxic drug/B cell depleting agent conjugates, wherein the B cell depleting agent is an antibody having specificity for antigenic determinants on B-cells. The present invention also relates to methods for producing the conjugates and to their therapeutic use(s). In particular, the present invention relates to methods for treating autoimmune diseases involving administering to a patient a B cell depleting agent, such as B-cell surface antigen targeting antibody (e.g., anti-CD22, anti-CD20, and/or anti-CD19 antibodies), or a conjugate of a B cell depleting agent with a cytotoxic drug. The present invention also relates to treatments for autoimmune diseases using B cell depleting agents, or conjugates of B cell depleting agents with cytotoxic drugs in combination with anti-cytokine agents such as anti-TNF agents.

### BACKGROUND OF THE INVENTION

Autoimmune diseases are a family of serious chronic illnesses in which the immune system mistakenly targets the cells, tissues and organs of an individual's own body. According to the National Institutes of Health, although many of the autoimmune diseases are indeed rare, as a group these diseases afflict millions of people in the United States alone. For reasons that are not well understood, autoimmune diseases strike women more often than men with about seventy five percent of cases occurring in women. In particular, these diseases most frequently affect women of working age and during their childbearing years. In fact, autoimmune disease represent the fourth largest cause of disability among women in the United States. Clearly, the social, economic and health impacts from autoimmune diseases are far-reaching.

The pathogenesis of autoimmune diseases involves a complicated network of tissue-damaging mechanisms that are governed primarily by recognition of self-antigens and an imbalance in cytokine production. Feldmann, M., Brennan, F. M. & Maini, R. N. Role of cytokines in rheumatoid arthritis. Annu Rev Immunol 14, 397-440 (1996). Marrack, P., Kappler, J. & Kotzin, B. L. Autoimmune disease: why and where it occurs. Nat Med 7, 899-905 (2001). In rheumatoid arthritis (RA), a common and debilitating autoimmune disease of still unknown etiology, major cell types responsible for chronic inflammation and subsequent cartilage destruction and bone erosion in the joints are macrophages, synovial fibroblasts, neutrophils, and lymphocytes.

Cytokines have also been implicated in autoimmune diseases. Cytokines are protein molecules that are released by cells when activated by antigens and are believed to be involved in cell-to-cell communications, acting as enhancing mediators for immune responses through interaction with specific cell-surface receptors on leukocytes. There are various different types of cytokines, including interleukins, lymphokines, interferons and tumor necrosis factor (TNF).

Currently available treatments for autoimmune diseases, such as antibody-based therapeutics, fail to effectively treat a variety of autoimmune diseases. Accordingly, there remains a significant need for an improved therapeutic approach to the treatment of autoimmune diseases. To fullfill this need, it would be useful to have a therapy that overcomes the shortcomings of current antibody-based therapeutics, treats a variety of autoimmune diseases, is produced easily and efficiently, and may be used repeatedly without inducing an immune response. There is also a need for a combination therapy that provides improved efficacy in treating autoimmune diseases, such as therapies that combine the use of an immunoconjugate with an anti-cytokine agent, such as an anti-TNF agent.

### SUMMARY OF THE INVENTION

An embodiment of the present invention provides a method for treating an autoimmune disease in a subject comprising: administering to the subject a therapeutically effective amount of: (a) a B cell depleting agent; and (b) at least one anti-cytokine agent.

A further embodiment of the present invention provides a method for treating an autoimmune disease in a subject comprising: administering to the subject a therapeutically effective amount of a monomeric cytotoxic drug/B cell depleting agent conjugate with reduced low conjugated fraction (LCF) having the formula,
Pr(—X—W)m
wherein:
Pr is a B cell depleting agent,
X is a linker that comprises a product of any reactive group that can react with a B cell depleting agent,
W is a cytotoxic drug;
m is the average loading for a purified conjugation product such that the cytotoxic drug constitutes 7-9% of the conjugate by weight; and (—X—W)m is a cytotoxic drug derivative.

An even further embodiment of the present invention provides a method of treating an autoimmune disease in a subject comprising: administering to the subject with the autoimmune disease a therapeutically effective amount of a monomeric calicheamicin derivative/anti-CD22 antibody conjugate having the formula,
Pr(—X—S—S—W)m
wherein:
Pr is an anti-CD22 antibody;
X is a hydrolyzable linker that comprises a product of any reactive group that can react with an antibody;
W is a calicheamicin radical;
m is the average loading for a purified conjugation product such that the calicheamicin constitutes 4-10% of the conjugate by weight; and (—X—S—S—W)m is a calicheamicin derivative.

A still further embodiment of the present invention provides a method of treating an autoimmune disease in a subject comprising administering a therapeutically effective amount of a stable lyophilized composition of a monomeric cytotoxic drug/B cell depleting agent conjugate, said conjugate being prepared by a method comprising: dissolving the monomeric cytotoxic drug/B cell depleting agent conjugate to a final concentration of 0.5 to 2 mg/mL in a solution comprising a cryoprotectant at a concentration of 1.5%-5% by weight, a polymeric bulking agent at a concentration of 0.5-1.5% by weight, electrolytes at a concentration of 0.01 M to 0.1 M, a solubility facilitating agent at a concentration of 0.005-0.05% by weight, buffering agent at a concentration of 5-50 mM such that the final pH of the solution is 7.8-8.2, and water; dispensing the above solution into vials at a temperature of +5° C. to +10° C.; freezing the solution at a freezing temperature of −35° C. to −50° C.; subjecting the frozen solution to an initial freeze drying step at a primary drying pressure of 20 to 80 microns at a shelf-temperature at −10° C. to −40° C. for 24 to 78 hours; and subjecting the freeze-dried product of step (d) to a secondary drying step at a drying pressure of 20 to 80 microns at a shelf temperature of +10° C. to +35° C. for 15 to 30 hours.

Another embodiment of the present invention provides a method for treating an autoimmune disease in a subject comprising: administering to the subject a therapeutically effective amount of a cytotoxic drug/B cell depleting agent conjugate, wherein said B cell depleting agent is an antibody.

Yet another embodiment of the present invention provides a method for treating an autoimmune disease in a subject comprising: administering to the subject a therapeutically effective amount of a B cell depleting agent, wherein the B cell depleting agent is a humanized antibody against CD22, CD19 or CD20.

A further embodiment of the present invention provides the use of a conjugate as described herein in the preparation of a medicament for the treatment of autoimmune disease in a subject comprising administering a therapeutically effective amount of said conjugate to a subject.

An even further embodiment of the present invention provides a composition comprising: (a) a cytotoxic drug/B cell depleting agent conjugate comprising at least one cytotoxic drug conjugated to at least one B cell depleting agent; and (b) at least one anti-cytokine agent.

### BRIEF DESCRIPTION OF THE DRAWINGS

FIG. 1 shows the amino acid sequence of the CDRs of mouse monoclonal antibody 5/44 (SEQ ID NOS:1 to 6).

FIG. 2 shows that Cy34.1 mAb conjugated to calicheamicin (CD22/cal) binds on B cells and inhibits proliferative responses following LPS stimulation. (a) structure of CD22/cal, a CD22-targeted immunoconjugate of calicheamicin. (b) A20 mouse B cell lymphoma cells were stained with Cy34.1 or CD22/cal immunoconjugate. (c) Proliferation of primary mouse B cells stimulated with LPS and incubated for 48 hr with increasing concentrations of Cy34.1 or CD22/cal. (d) Proliferation of primary mouse B cells stimulated with LPS and incubated for 48 hr with increasing concentrations of CD22/cal or control J110/cal antibody. (e) Proliferation of primary mouse T cells to TCR costimulation after incubation for 48 hr with increasing concentrations of CD22/cal or control J110/cal Ab.

FIG. 3 illustrates the in-vivo cytotoxic effect of CD22/cal immunocomjugate. (a) Percentages of CD22⁺ B cells in PB, spleen, BM, and LN before (Pre) and 12 days after (After) two injections with CD22/cal. (b) Day 12 samples were also stained for CD19 expression. (c) The indicated tissue samples from untreated wt B6 mice were double-stained for the expression of CD22 and CD19.

FIG. 4 illustrates the in-vivo effect of CD22/cal immunoconjugate on CD3⁺ T cells and Gr-1⁺ myeloid cells. Percentages of CD3⁺ T cells (a) and Gr-1⁺ myeloid cells (b) in PB, spleen, BM, and LN samples before (Pre) and 12 days after (After) two injections with CD22/cal. (c) The indicated tissues from mice injected with CD22/cal on days 0 and 5 were collected on day 50 and stained for CD22 expression.

FIG. 5 illustrates that B cell depletion with CD22/cal immunoconjugate inhibits the development of clinical arthritis. Groups of B6 IFN-γ KO mice were immunized on day 0 with collagen II in CFA and injected on days 5 and 10 with PBS (a) or CD22/cal (b). Paws were evaluated for clinical arthritis using a semi-quantitative scoring system. A representative experiment of two performed is shown.

FIG. 6 illustrates that B cell depletion with CD22/cal immunoconjugate inhibits histological signs of arthritis. Groups of B6 IFN-γ KO mice were immunized on day 0 with collagen II in CFA and injected on days 5 and 10 with PBS (untreated) or CD22/cal (B-cell depleted). Paws for histopathological evaluation were collected from two different experiments on day 25 (a, b) or day 75 (c, d) after immunization with collagen II.

FIG. 7 demonstrates that administration of CD22/cal does not alter anti-F protein antibody titers in B6 mice immunized with the F protein of RSV. (a) Serum IgM and (b) serum IgG titers in B6 mice (F/AIPO) immunized on week 0 and 2 (black arrows) with F protein. Control mice (PBS) were not immunized. On weeks 4 and 4 plus 5 days (white arrows) F/AIPO and PBS mice received CD22/cal or were administered PBS alone. All mice were administered infectious RSV (*) on week 12.

### DETAILED DESCRIPTION OF THE INVENTION

For purposes of this disclosure, the terms “illness,” “disease,” medical disorder,” “medical condition,” “abnormal condition” and the like are used interchangeably.

The term “B cell depleting agent,” as used herein, refers to any agent (e.g., antibody, antagonist, etc.) that reduces B cell circulating levels in an organism or that reduces or interferes with the activity of B cells in an organism.

As used herein, the term “cytotoxic drug/B cell depleting agent conjugate” describes any construct comprising any cytotoxic drug, cytotoxic drug derivative and the like conjugated to any B cell depleting agent and the like in any manner as known to persons skilled in the art. As used in this expression, the term “cytotoxic drug” is used interchangeably with the term “cytotoxic drug derivative”. This contemplates that the cytotoxic drug in the conjugate may be a derivatized version of the cytotoxic drug used to prepare the conjugate.

The term “anti-cytokine agent,” as used herein, refers to any agent that reduces the activity of a cytokine, e.g., tumor necrosis factors (TNF), interleukins, lymphokines, interferons, and especially an agent that binds to a cytokine.

The term “isolated” or “purified”, as used in the context of this specification to define the purity of compositions, such as protein compositions, means that the composition is substantially free of other components of natural or endogenous origin and contains less than about 1% by mass of contaminants residual of production processes. Such compositions, however, can contain other proteins added as stabilizers, carders, excipients or co-therapeutics. For example, TNFR is considered isolated if it is detectable as a single protein band in a polyacrylamide gel by silver staining.

“Recombinant,” as used herein, means that a protein is derived from recombinant (e.g., microbial or mammalian) expression systems. “Microbial” refers to recombinant proteins made in bacterial or fungal (e.g., yeast) expression systems. As a product, “recombinant microbial” defines a protein produced in a microbial expression system which is essentially free of native endogenous substances. Protein expressed in most bacterial cultures, e.g., E. coli, will be free of glycan. Protein expressed in yeast may have a glycosylation pattern different from that expressed in mammalian cells.

“Biologically active,” as used throughout the specification as a characteristic of protein receptors, e.g., TNF receptors, means that a particular molecule shares sufficient amino acid sequence similarity with the embodiments of the present invention disclosed herein to be capable of binding detectable quantities of protein e.g., TNF, transmitting a protein stimulus to a cell, for example, as a component of a hybrid receptor construct, or cross-reacting with antibodies against the protein, e.g., anti-TNFR antibodies raised against TNFR, from natural (i.e., nonrecombinant) sources. Preferably, biologically active TNF receptors within the scope of the present invention are capable of binding greater than 0.1 nmoles TNF per nmole receptor, and most preferably, greater than 0.5 nmole TNF per nmole receptor in standard binding assays.

As used herein, the term “antigen binding region” refers to that portion of an antibody molecule which contains the amino acid residues that interact with an antigen and confer on the antibody its specificity and affinity for the antigen. The antibody region includes the “framework” amino acid residues necessary to maintain the proper conformation of the antigen-binding residues.

As used herein, the term “chimeric antibody” includes monovalent, divalent or polyvalent immunoglobulins. A monovalent chimeric antibody is a dimer (HL)) formed by a chimeric H chain associated through disulfide bridges with a chimeric L chain. A divalent chimeric antibody is tetramer (H₂L₂) formed by two HL dimers associated through at least one disulfide bridge. A polyvalent chimeric antibody can also be produced, for example, by employing a C_{H} region that aggregates (e.g., from an IgM H chain, or .mu. chain).

The phrase “therapeutically effective amount,” as used herein, refers to the amount to be administered to a subject (preferably human) in each single dose or as part of a series of doses to at least cause the individual treated to generate a response that reduces the clinical impact of the condition being treated. The dosage amount can vary depending upon specific conditions of the individual. The specific amount to administer can be determined in routine trials or otherwise by means known to those skilled in the art, based upon the guidance provided herein.

As used herein, the phrase “administering a therapeutically effective amount” of a therapeutic agent means that the patient is treated with the agent in an amount and for a time sufficient to induce a sustained improvement over baseline in at least one indicator that reflects the severity of the disorder. An improvement is considered “sustained” if the patient exhibits the improvement on at least two occasions separated by one or more weeks. The degree of improvement is determined based on signs or symptoms, and determinations may also employ questionnaires that are administered to the patient, such as quality-of-life questionnaires.

As used herein, the terms “tumor necrosis factor” or “TNF” refer to TNF-alpha and/or TNF-beta.

The terms “TNF receptor” and “TNFR” refer to proteins having amino acid sequences which are substantially similar to the native mammalian TNF receptor or TNF binding protein amino acid sequences, and which are capable of binding TNF molecules and inhibiting TNF from binding to cell membrane bound TNFR.

A novel mouse B cell-targeted cytotoxic immunoconjugate (anti-CD22 mAb antibody conjugated to calicheamicin) was developed to study by flow cytometric analysis the characteristics of B cell depletion and recovery in peripheral blood (PB), spleen, bone marrow (BM), and lymph node (LN) samples from naïve mice. The study showed the effects of B cell depletion on the development of clinical and histological arthritis in a mouse collagen-induced arthritis (CIA) model and on humoral immune responses in the mouse model of RSV infection. The results of these studies show that depletion of B cells with two injections of immunoconjugate inhibits clinical and histological arthritis in the CIA model, whereas the same protocol does not adversely affect memory antibody responses after challenge and clearance of infectious virus from lungs in the RSV vaccination model. These results provide novel insights into the role of CD22-targeted B cell depletion in mouse autoimmunity and vaccination models.

The present invention is directed to compositions and methods that are effective in treating autoimmune diseases. In particular, the present invention provides B cell depleting agents (e.g., humanized antibodies), cytotoxic drug/B cell depleting agent conjugates, anti-cytokine agents (e.g., anti-TNF agents), and combinations thereof.

The conjugates of the present invention comprise a B cell depleting agent, such as an antibody or preferably a humanized antibody. The invention relates to conjugates of antibodies and cytotoxic drugs, wherein the antibody has specificity for antigenic determinants on B-cells. The present invention also relates to methods for producing immunoconjugates and to their therapeutic use(s).

Anti-cytokine agents may be used in combination with the B cell depleting agents and/or cytotoxic drugs of the present invention. The present invention contemplates the use of anti-cytokine agents in combination with the cytotoxic drug/B cell depleting agent conjugates of the present invention. The present invention provides compositions comprising therapeutically effective amounts of an anti-cytokine agent, alone or in combination with the B cell depleting agent, cytotoxic drug or conjugates of same, preferably in a suitable drug delivery system, such as a pharmaceutically acceptable diluent. The present invention provides methods of using said compositions for treating autoimmune diseases. Based upon the guidance provided herein, a person of skill in the art would readily be able to identify such a compound or composition, in accordance with an implementation of the invention.

The conjugates of the present invention can be administered alone or in combination with one or more compounds of the invention or other agents, such as anti-cytokine agents, as described herein. The agents can be formulated as separate compositions that are administered at the same time or sequentially at different times, or the agents can be given in a single composition, as described herein.

The conjugates of the present invention preferably comprise a cytotoxic drug derivatized with a linker that includes any reactive group that reacts with a B cell depleting agent to form a cytotoxic drug/B cell depleting agent conjugate. Specifically, the conjugates of the present invention comprise a cytotoxic drug derivatized with a linker that includes any reactive group which reacts with an antibody used as a B cell depleting agent to form a cytotoxic drug/antibody conjugate. Specifically, the antibody reacts against a cell surface antigen expressed on certain B-cells. Described below is an improved process for making and purifying such conjugates. The use of particular cosolvents, additives, and specific reaction conditions together with the separation process results in the formation of a monomeric cytotoxic drug/antibody conjugate with a significant reduction in the low conjugated fraction (LCF). The monomeric form as opposed to the aggregated form has significant therapeutic value, and minimizing the LCF and substantially reducing aggregation results in the utilization of the antibody starting material in a therapeutically meaningful manner by preventing the LCF from competing with the more highly conjugated fraction (HCF).

B Cell Depleting Agents

The present invention provides B cell depleting agents having specificity for cell surface antigenic determinants. The B cell depleting agents may be administered as part of a composition in combination with other agents, such as cytotoxic drugs and/or anti-cytokine agents, or alone, and optionally with a pharmaceutically acceptable diluent. The B cell depleting agents may be administered as part of a monotherapy or a combination therapy with cytotoxic drugs, anti-cytokine agents and/or other agents.

B cell depleting agents include hormones, growth factors, antibodies, antibody fragments, antibody mimics, and their genetically or enzymatically engineered counterparts, hereinafter referred to singularly or as a group as “B cell depleting agents”. Preferably, the B cell depleting agent has the ability to recognize and bind to an antigen or receptor associated with certain cells and to be subsequently internalized. Examples of B cell depleting agents that are applicable in the present invention are disclosed in U.S. Pat. No. 5,053,394, which is incorporated herein in its entirety. Preferred B cell depleting agents for use in the present invention are antibodies and antibody mimics.

The antibodies contemplated by the present invention include effector antibodies which do not need to bind to an internalizing receptor to destroy or interfere with a target cell and antibodies that do need to bind to an internalizing receptor to destroy or interfere with the cell. Preferably, antibodies that need to bind to an internalizing receptor are conjugated to a cytotoxic agent.

The present invention provides humanized antibodies as B cell depleting agents, and compositions comprising the humanized antibodies. Also contemplated are methods of administering to a patient a therapeutically effective amount of the humanized antibodies described herein for treatment of autoimmune diseases.

A number of non-immunoglobulin protein scaffolds have been used for generating antibody mimics that bind to antigenic epitopes with the specificity of an antibody (PCT publication No. WO 00/34784). For example, a “minibody” scaffold, which is related to the immunoglobulin fold, has been designed by deleting three beta strands from a heavy chain variable domain of a monoclonal antibody (Tramontano et al., J. Mol. Recognit. 7:9, 1994). This protein includes 61 residues and can be used to present two hypervariable loops. These two loops have been randomized and products selected for antigen binding, but thus far the framework appears to have somewhat limited utility due to solubility problems. Another framework used to display loops is tendamistat, a protein that specifically inhibits mammalian alpha-amylases and is a 74 residue, six-strand beta-sheet sandwich held together by two disulfide bonds, (McConnell and Hoess, J. Mol. Biol. 250:460, 1995). This scaffold includes three loops, but, to date, only two of these loops have been examined for randomization potential.

Other proteins have been tested as frameworks and have been used to display randomized residues on alpha helical surfaces (Nord et al., Nat. Biotechnol. 15:772, 1997; Nord et al., Protein Eng. 8:601, 1995), loops between alpha helices in alpha helix bundles (Ku and Schultz, Proc. Natl. Acad. Sci. USA 92:6552, 1995), and loops constrained by disulfide bridges, such as those of the small protease inhibitors (Markland et al., Biochemistry 35:8045, 1996; Markland et al., Biochemistry 35:8058, 1996; Rottgen and Collins, Gene 164; 243, 1995; Wang et al., J. Biol. Chem. 270:12250, 1995).

Examples of B cell depleting agents that may be used in the present invention include monoclonal antibodies, chimeric antibodies, humanized antibodies, human antibodies and biologically active fragments thereof. Preferably, such antibodies are directed against cell surface antigens expressed on target cells. Examples of specific antibodies directed against cell surface antigens on target cells include without limitation, antibodies against CD22 antigen which is over-expressed on most B-cell lymphomas; G5/44, a humanized form of a murine anti-CD22 monoclonal antibody. In addition, there are several commercially available antibodies such as rituximab (Rituxan™), which may also be used as B cell depleting agent.

Exemplified herein for use as a B cell depleting agent in the present invention is a CDR-grafted humanized antibody molecule directed against cell surface antigen CD22, designated G5/44. This antibody is a humanized form of a murine anti-CD22 monoclonal antibody that is directed against the cell surface antigen CD22, which is prevalent on certain human lymphomas. The term “a CDR-grafted antibody molecule” as used herein refers to an antibody molecule wherein the heavy and/or light chain contains one or more complementarity determining regions (CDRs) including, if desired, a modified CDR (hereinafter CDR) from a donor antibody (e.g., a murine monoclonal antibody) grafted into a heavy and/or light chain variable region framework of an acceptor antibody (e.g., a human antibody). Preferably, such a CDR-grafted antibody has a variable domain comprising human acceptor framework regions as well as one or more of the donor CDRs referred to above.

When the CDRs are grafted, any appropriate acceptor variable region framework sequence may be used having regard to the class/type of the donor antibody from which the CDRs are derived, including mouse, primate and human framework regions. Examples of human frameworks, which can be used in the present invention are KOL, NEWM, REI, EU, TUR, TEI, LAY and POM (Kabat et al. Seq. of Proteins of Immunol. Interest, 1:310-334 (1994)). For example, KOL and NEWM can be used for the heavy chain, REI can be used for the light chain and EU, LAY and POM can be used for both the heavy chain and the light chain.

In a CDR-grafted antibody of the present invention, it is preferred to use as the acceptor antibody one having chains which are homologous to the chains of the donor antibody. The acceptor heavy and light chains do not necessarily need to be derived from the same antibody and may, if desired, comprise composite chains having framework regions derived from different chains.

Also, in a CDR-grafted antibody of the present invention, the framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody or to a residue that is a conservative substitution for the residue found at the same position in the donor antibody. Such changes should be kept to the minimum necessary to recover the affinity of the donor antibody. A protocol for selecting residues in the acceptor framework regions which may need to be changed is set forth in PCT Publication No. WO 91/09967, which is incorporated herein in its entirety.

Donor residues are residues from the donor antibody, i.e., the antibody from which the CDRs were originally derived.

The antibody of the present invention may comprise a heavy chain wherein the variable domain comprises as CDR-H2 (as defined by Kabat et al., (supra)) an H2′ in which a potential glycosylation site sequence has been removed in order to increase the affinity of the antibody for the antigen.

Alternatively or additionally, the antibody of the present invention may comprise a heavy chain wherein the variable domain comprises as CDR-H2 (as defined by Kabat et al., (supra)) an H2″ in which a lysine residue is at position 60. This lysine residue, which is located at an exposed position within CDR-H2, and is considered to have the potential to react with conjugation agents resulting in a reduction of antigen binding affinity, is substituted with an alternative amino acid.

Additionally, the antibody of the present invention may comprise a heavy chain wherein the variable domain comprises as CDR-H2 (as defined by Kabat et al., (supra)) an H2′″ in which both the potential glycosylation site sequence and the lysine residue at position 60, are substituted with alternative amino acids.

The antibody of the present invention may comprise: a complete antibody having full length heavy and light chains; a biologically active fragment thereof, such as a Fab, modified Fab, Fab′, F(ab′)₂ or Fv fragment; a light chain or heavy chain monomer or dimer; or a single chain antibody, e.g., a single chain Fv in which the heavy and light chain variable domains are joined by a peptide linker. Similarly, the heavy and light chain variable regions may be combined with other antibody domains as appropriate.

The antibody of the present invention may also include a modified Fab fragment wherein the modification is the addition of one or more amino acids to allow for the attachment of an effector or reporter molecule to the C-terminal end of its heavy chain. Preferably, the additional amino acids form a modified hinge region containing one or two cysteine residues to which the effector or reporter molecule may be attached.

The constant region domains of the antibody of the present invention, if present, may be selected having regard to the proposed function of the antibody, and in particular the effector functions which may or may not be required. For example, the constant region domains may be human IgA, IgD, IgE, IgG or IgM domains. In particular, human IgG constant region domains may be used, especially of the IgG1 and IgG3 isotypes when the antibody is intended for therapeutic uses and antibody effector functions are required. Alternatively, IgG2 and IgG4 isotypes may be used or the IgG1 Fc region may be mutated to abrogate the effector function when the antibody is intended for therapeutic purposes and antibody effector functions are not required or desired.

The antibody of the present invention has a binding affinity of at least 5×10⁸ M, preferably at least 1×10⁻⁹ M, more preferably at least 0.75×10⁻¹⁰ M, and most preferably at least 0.5×10⁻¹⁰ M.

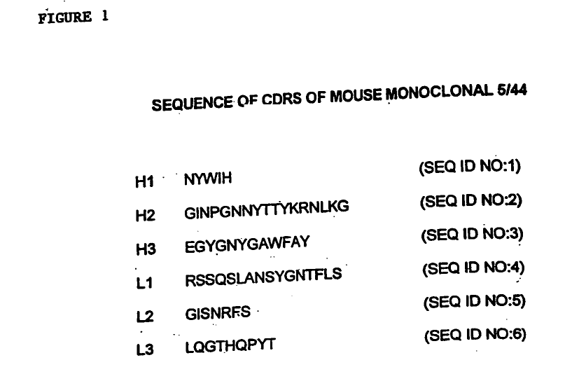

Nonlimiting exemplary B cell depleting agents of the present invention include the following: an anti-CD22 antibody that has specificity for human CD22, and comprises a heavy chain wherein the variable domain comprises a CDR having at least one of the sequences given as H1 in FIG. 1 (SEQ ID NO:1) for CDR-H1, as H2 in FIG. 1 (SEQ ID NO:2) or H2′ (SEQ ID NO:13) or H2″ (SEQ ID NO:15) or H2′″ (SEQ ID NO:16) for CDR-H2, or as H3 in FIG. 1 (SEQ ID NO:3) for CDR-H3, and comprises a light chain wherein the variable domain comprises a CDR having at least one of the sequences given as L1 in FIG. 1 (SEQ ID NO:4) for CDR-L1, as L2 in FIG. 1 (SEQ ID NO:5) for CDR-L2, or as L3 in FIG. 1 (SEQ ID NO:6) for CDR-L3; an anti-CD22 antibody comprising a heavy chain wherein the variable domain comprises a CDR having at least one of the sequences given in SEQ ID NO:1 for CDR-H1, SEQ ID NO:2 or SEQ ID NO:13 or SEQ ID NO:15 or SEQ ID NO:16 for CDR-H2, or SEQ ID NO:3 for CDR-H3, and a light chain wherein the variable domain comprises a CDR having at least one of the sequences given in SEQ ID NO:4 for CDR-L1, SEQ ID NO:5 for CDR-L2, or SEQ ID NO:6 for CDR-L3; an anti-CD22 antibody comprising SEQ ID NO:1 for CDR-H1, SEQ ID NO: 2 or SEQ ID NO:13 or SEQ ID NO:15 or SEQ ID NO:16 for CDR-H2, SEQ ID NO:3 for CDR-H3, SEQ ID NO:4 for CDR-L1, SEQ ID NO:5 for CDR-L2, and SEQ ID NO:6 for CDR-L3; a humanized anti-CD22 antibody that is a CDR-grafted anti-CD22 antibody and comprises a variable domain comprising human acceptor framework regions and non-human donor CDRs; a humanized anti-CD22 antibody that has a human acceptor framework wherein regions of the variable domain of the heavy chain of the antibody are based on a human sub-group I consensus sequence and comprise non-human donor residues at positions 1, 28, 48, 71 and 93; a humanized antibody as described above that further comprises non-human donor residues at positions 67 and 69; a CDR-grafted humanized antibody comprising a variable domain of the light chain comprising a human acceptor framework region based on a human sub-group I consensus sequence and further comprising non-human donor residues at positions 2, 4, 37, 38, 45 and 60; a CDR-grafted antibody as previously described further comprising a non-human donor residue at position 3; a CDR-grafted antibody as previously described comprises a light chain variable region 5/44-gL1 (SEQ ID NO:19) and a heavy chain variable region 5/44-gH7 (SEQ ID NO:27); a CDR-grafted antibody comprising a light chain having the sequence as set forth in SEQ ID NO: 28 and a heavy chain having the sequence as set forth in SEQ ID NO:30; a CDR-grafted antibody comprising a light chain having the sequence as set forth in SEQ ID NO: 28 and a heavy chain having the sequence as set forth in SEQ ID NO: 30; an anti-CD22 CDR-grafted antibody that is a variant antibody obtained by an affinity maturation protocol and has increased specificity for human CD22; an anti-CD22 antibody that is a chimeric antibody comprising the sequences of the light and heavy chain variable domains of the monoclonal antibody set forth in SEQ ID NO:7 and SEQ ID NO:8, respectively; an anti-CD22 antibody comprising a hybrid CDR with a truncated donor CDR sequence wherein the missing portion of the donor CDR is replaced by a different sequence and forms a functional CDR.

Preferably, the humanized anti-CD22 antibodies of the present invention is a CDR-grafted antibody comprising a light chain variable region 5/44-gL1 (SEQ ID NO:19), and a heavy chain variable region 5/44-gH7 (SEQ ID NO:27), a CDR-grafted antibody comprising a light chain having a sequence set forth in SEQ ID NO: 28, a CDR-grafted antibody comprising a heavy chain having a sequence set forth in SEQ ID NO:30, a CDR-grafted antibody comprising a light chain having a sequence set forth in SEQ ID NO: 28 and a heavy chain having a sequence set forth in SEQ ID NO: 30, or a CDR-grafted antibody that is a variant antibody obtained by an affinity maturation protocol and has increased specificity for human CD22.

The present invention contemplates the use of recombinant (or recombinantly prepared) proteins or polypeptides, as B cell depleting agents. For example, a recombinant polypeptide or protein of the invention may be a recombinant that is identical to the reference sequence herein that is, 100% identical, or it may include a number of amino acid alterations as compared to the reference sequence such that the % identity is less than 100%. Such alterations include at least one amino acid deletion, substitution, including conservative and non-conservative substitution, or insertion. The alterations may occur at the amino- or carboxy-terminal positions of the reference polypeptide sequence or anywhere between those terminal positions, interspersed either individually among the amino acids in the reference amino acid sequence or in one or more contiguous groups within the reference amino acid sequence.

Thus, the invention also provides proteins having sequence identity to the amino acid sequences contained in the Sequence Listing. Depending on the particular sequence, the degree of sequence identity is preferably greater than 60% (e.g., 60%, 70%, 80%, 90%, 95%, 97%, 99%, 99.9% or more). These homologous proteins include mutants and allelic variants. The polypeptide may be any fragment or biological equivalent of the listed polypeptides.

This invention also relates to allelic or other variants of the polypeptides, which are biological equivalents. Suitable biological equivalents have at least about 60%, preferably at least about 70%, more preferably at least about 75%, even more preferably about 80%, even more preferably about 85%, even more preferably about 90%, even more preferably 95% or even more preferably 98%, or even more preferably 99% similarity to one of the proteins or polypeptides specified herein (i.e., provided the equivalent is capable of eliciting substantially the same immunogenic properties as one of the proteins of this invention).

The biological equivalents are obtained by generating variants and modifications to the proteins of this invention. These variants and modifications to the proteins are obtained by altering the amino acid sequences by insertion, deletion or substitution of one or more amino acids. The amino acid sequence is modified, for example by substitution in order to create a polypeptide having substantially the same or improved qualities. A preferred means of introducing alterations comprises making predetermined mutations of the nucleic acid sequence of the polypeptide by site-directed mutagenesis.

Modifications and changes can be made in the structure of a protein or polypeptide of the present invention (e.g., carrier, antibody, humanized antibody, etc.) while retaining functional equivalency (such as immunogenicity, therapeutic benefit, binding affinity, etc). Such modifications and changes are fully contemplated by the present invention. For example, without limitation, certain amino acids can be substituted for other amino acids, including nonconserved and conserved substitution, in a sequence without appreciable loss of functionality/utility (e.g., immunogenicity, therapeutic benefit, etc.). Because it is the interactive capacity and nature of a polypeptide that defines that polypeptide's biological functional activity, a number of amino acid sequence substitutions can be made in a polypeptide sequence (or, of course, its underlying DNA coding sequence) and nevertheless obtain a polypeptide with like properties. The present invention contemplates any changes to the structure of the polypeptides herein, as well as the nucleic acid sequences encoding said polypeptides, wherein the polypeptide retains its functionality or a biologically equivalent functionality. A person of ordinary skill in the art would be readily able to routinely modify the disclosed polypeptides and polynucleotides accordingly, based upon the guidance provided herein, while remaining consistent with the inventive concept and the purposes of the present invention.

In making such changes, any techniques known to persons of skill in the art may be utilized. For example, without intending to be limited thereto, the hydropathic index of amino acids can be considered. The importance of the hydropathic amino acid index in conferring interactive biologic function on a polypeptide is generally understood in the art. Kyte et al. 1982. J. Mol. Bio. 157:105-132.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its functionality, i.e. with a biological property of the polypeptide.

Biological equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. Such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the polypeptide sequence selected. An oligonucleotide primer bearing the desired mutated sequence is prepared (e.g., synthetically). This primer is then annealed to the single-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

The polypeptides of the invention include any protein or polypeptide comprising substantial sequence similarity and/or biological equivalence to a protein having an amino acid sequence from one of the specifically identified sequences herein. In addition, the polypeptides of the invention are not limited to a particular source. Also, the polypeptides can be prepared recombinantly using any such technique in accordance with the purpose of the invention as described herein, as is well within the skill in the art, based upon the guidance provided herein, or in any other synthetic manner, as known in the art.

It is contemplated in the present invention, that a polypeptide may advantageously be cleaved into fragments for use in further structural or functional analysis, or in the generation of reagents such as related polypeptides and specific antibodies. This can be accomplished by treating purified or unpurified polypeptides with a peptidase such as endoproteinase glu-C (Boehringer, Indianapolis, Ind.). Treatment with CNBr is another method by which peptide fragments may be produced from polypeptides. Recombinant techniques also can be used to produce specific fragments of a protein.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical (i.e., biologically equivalent). A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al 1984), BLASTP, BLASTN, and FASTA (Altschul, S. F., et al., 1990). The BLASTX program is publicly available from NCBI and other sources (BLAST Manual, Altschul, S., et al., NCBI NLM NIH Bethesda, Md. 20894; Altschul, S., et al., 1990). The well known Smith Waterman algorithm may also be used to determine identity.

By way of example, without intending to be limited thereto, an amino acid sequence of the present invention may be identical to any specifically identified sequence provided herein; that is be 100% identical, or it may include a number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids by the numerical percent of the respective percent identity (divided by 100) and then subtracting that product from said total number of amino acids, or:
nₐ=xₐ−(xₐ·y),
wherein nₐ is the number of amino acid alterations, xₐ is the total number of amino acids, and y is, for instance 0.70 for 70%, 0.80 for 80%, 0.85 for 85% etc., and wherein any non-integer product of xₐ and y is rounded down to the nearest integer prior to subtracting it from xₐ.

In one embodiment, the present invention relates to immunotoxin conjugates and methods for making these conjugates using antibody variants or antibody mimics. In a preferred embodiment, variants of the antibody of the present invention are directed against CD22 and display improved affinity for CD22. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998).

Any suitable host cell/vector system may be used for expression of the DNA sequences encoding the B cell depleting agent including antibodies of the present invention. Bacterial, for example E. coli, and other microbial systems may be used, in part, for expression of antibody fragments such as Fab and F(ab′)₂ fragments, and especially Fv fragments and single chain antibody fragments, for example, single chain Fvs. Eukaryotic, e.g. mammalian, host cell expression systems may be used for production of larger antibody, including complete antibody molecules. Suitable mammalian host cells include CHO, myeloma, yeast cells, insect cells, hybridoma cells, NSO, VERO or PER C6 cells. Suitable expression systems also include transgenic animals and plants.

Cytotoxic Drugs

The present invention provides cytotoxic drugs, compositions comprising cytotoxic drugs, such as cytotoxic drug/B cell depleting agent conjugates, and therapies involving administration of cytotoxic drugs for the treatment of autoimmune diseases.

The cytotoxic drugs suitable for use in the present invention are cytotoxic drugs that inhibit or disrupt tubulin polymerization, alkylating agents that bind to and disrupt DNA, and agents which inhibit protein synthesis or essential cellular proteins such as protein kinases, enzymes and cyclins. Examples of such cytotoxic drugs include, but are not limited to thiotepa, taxanes, vincristine, daunorubicin, doxorubicin, epirubicin, actinomycin, authramycin, azaserines, bleomycins, tamoxifen, idarubicin, dolastatins/auristatins, hemiasterlins, calicheamicins, esperamicins and maytansinoids. Preferred cytotoxic drugs are the calicheamicins, which are an example of the methyl trisulfide antitumor antibiotics. Examples of calicheamicins suitable for use in the present invention are disclosed, for example, in U.S. Pat. No. 4,671,958; U.S. Pat. No. 4,970,198, U.S. Pat. No. 5,053,394, U.S. Pat. No. 5,037,651; and U.S. Pat. No. 5,079,233, which are incorporated herein in their entirety. Preferred calicheamicins are the gamma-calicheamicin derivatives or the N-acetyl gamma-calicheamicin derivatives.

Cytotoxic Drug/B Cell Depleting Agent Conjugates

The present invention provides cytotoxic drug/B cell depleting agent conjugates comprising a cytotoxic drug and a B cell depleting agent. The present invention contemplates the use and preparation of any suitable conjugate of a B cell depleting agent and cytotoxic drug as would be known to persons skilled in the art. Exemplary B cell depleting agents, cytotoxic drug/B cell depleting agent conjugates and methods for preparing same are described in U.S. Patent Application No. US 2004/0082764 and PCT publication WO 03/092623 which are herein incorporated by reference in their entirety.

Preferably, the cytotoxic drug/B cell depleting agent conjugates of the present invention have the formula:
Pr(—X—W)m
wherein:
Pr is a B cell depleting agent,
X is a linker that comprises a product of any reactive group that can react with a B cell depleting agent,
W is the cytotoxic drug;
m is the average loading for a purified conjugation product such that the calicheamicin constitutes 4-10% of the conjugate by weight; and
(—X—W)m is a cytotoxic drug

Preferably, X has the formula
(CO-Alk¹-Sp¹-Ar-Sp²-Alk²-C(Z¹)=Q-Sp)
wherein
Alk¹ and Alk² are independently a bond or branched or unbranched (C₁-C₁₀) alkylene chain;
Sp¹ is a bond, —S—, —O—, —CONH—, —NHCO—, —NR′—, —N(CH₂CH₂)₂N—, or —X—Ar′—Y—(CH₂)ₙ-Z wherein X, Y, and Z are independently a bond, —NR′—, —S—, or —O—, with the proviso that when n=0, then at least one of Y and Z must be a bond and Ar′ is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR′, —CONHR′, —(CH₂)ₙCOOR′, —S(CH₂)ₙCOOR′, —O(CH₂)ₙCONHR′, or —S(CH₂)ₙCONHR′, with the proviso that when Alk¹ is a bond, Sp¹ is a bond;
n is an integer from 0 to 5;
R′ is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined or a 1,2-, 1,3-, 1,4-, 1,5-, 1,6-, 1,7-, 1,8-, 2,3-, 2,6-, or 2,7-naphthylidene or 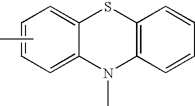
with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, or —S(CH₂)ₙCONHR′ wherein n and R′ are as defined above, with the proviso that when Ar is phenothiazine, Sp¹ is a bond only connected to nitrogen;
Sp² is a bond, —S—, or —O—, with the proviso that when Alk² is a bond, Sp² is a bond;
Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR′, —ONHR′, —O(CH₂)ₙCOOR′, —S(CH₂)ₙCOOR′, —O(CH₂)ₙCONHR′, or —S(CH₂)ₙCONHR′ wherein n and R′ are as defined above;
Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp can be additionally substituted by lower (C₁-C₅) dialkylamino, lower (C₁-C₅) alkoxy, hydroxy, or lower (C₁-C₅) alkylthio groups; and

Q is ═NHNCO—, ═NHNCS—, ═NHNCONH—, ═NHNCSNH—, or ═NHO—.

Preferably, Alk¹ is a branched or unbranched (C₁-C₁₀) alkylene chain; Sp¹ is a bond, —S—, —O—, —CONH—, —NHCO—, or —NR′ wherein R′ is as hereinbefore defined, with the proviso that when Alk¹ is a bond, Sp¹ is a bond;

Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′.

Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′; Alk² and Sp² are together a bond; and Sp and Q are as immediately defined above.

U.S. Pat. No. 5,773,001, incorporated herein in its entirety, discloses linkers that can be used with nucleophilic derivatives, particularly hydrazides and related nucleophiles, prepared from the calicheamicins. These linkers are especially useful in those cases where better activity is obtained when the linkage formed between the drug and the linker is hydrolyzable. These linkers contain two functional groups. One group typically is a carboxylic acid that is utilized to react with the B cell depleting agent. The acid functional group, when properly activated, can form an amide linkage with a free amine group of the B cell depleting agent, such as, for example, the amine in the side chain of a lysine of an antibody or other B cell depleting agent. The other functional group commonly is a carbonyl group, i.e., an aldehyde or a ketone, which will react with the appropriately modified therapeutic agent. The carbonyl groups can react with a hydrazide group on the drug to form a hydrazone linkage. This linkage is hydrolyzable, allowing for release of the therapeutic agent from the conjugate after binding to the target cells.

A most preferred bifunctional linker for use in the present invention is 4-(4-acetylphenoxy) butanoic acid (AcBut), which results in a preferred product wherein the conjugate consists of β-calicheamicin, γ-calicheamicin or N-acetyl γ-calicheamicin functionalized by reacting with 3-mercapto-3-methyl butanoyl hydrazide, the AcBut linker, and a human or humanized IgG antibody targeting B cell depleting agent.

Monomeric Conjugation

The natural hydrophobic nature of many cytotoxic drugs including the calicheamicins creates difficulties in the preparation of monomeric drug conjugates with good drug loadings and reasonable yields which are necessary for therapeutic applications. The increased hydrophobicity of the linkage provided by linkers, such as the AcBut linker, disclosed in U.S. Pat. No. 5,773,001, as well as the increased covalent distance separating the therapeutic agent from the B cell depleting agent (antibody), exacerbate this problem.

Aggregation of cytotoxic drug/B cell depleting agent conjugates with higher drug loadings occurs due to the hydrophobic nature of the drugs. The drug loading often has to be limited to obtain reasonable quantities of monomeric product. In some cases, such as with the conjugates in U.S. Pat. No. 5,877,296, it is often difficult to make conjugates in useful yields with useful loadings for therapeutic applications using the reaction conditions disclosed in U.S. Pat. No. 5,053,394 due to excessive aggregation. These reaction conditions utilized DMF as the co-solvent in the conjugation reaction. Methods which allow for higher drug loadings/yield without aggregation and the inherent loss of material are therefore needed.

Improvements to reduce aggregation are described in U.S. Pat. Nos. 5,712,374 and 5,714,586, which are incorporated herein in their entirety. Disclosed in those patents are B cell depleting agents including, but not limited to, proteins such as human or humanized antibodies that are used to target the cytotoxic therapeutic agents, such as, for example, hP67.6 and the other humanized antibodies disclosed therein. In those patents, the use of a non-nucleophilic, protein-compatible, buffered solution containing (i) propylene glycol as a cosolvent and (ii) an additive comprising at least one C₆-C₁₈ carboxylic acid was found to generally produce monomeric cytotoxic drug derivative derivative/B cell depleting agent conjugates with higher drug loading/yield and decreased aggregation having excellent activity. Preferred acids described therein were C₇ to C₁₂ acids, and the most preferred acid was octanoic acid (such as caprylic acid) or its salts. Preferred buffered solutions for conjugates made from N-hydroxysuccinimide (OSu) esters or other comparably activated esters were phosphate-buffered saline (PBS) or N-2-hydroxyethyl piperazine-N′-2-ethanesulfonic acid (HEPES buffer). The buffered solution used in those conjugation reactions cannot contain free amines or nucleophiles. For other types of conjugates, acceptable buffers can be readily determined. Alternatively, the use of a non-nucleophilic, protein-compatible, buffered solution containing t-butanol without the additional additive was also found to produce monomeric calicheamicin derivative/B cell depleting agent conjugates with higher drug loading/yield and decreased aggregation.

The amount of cosolvent needed to form a monomeric conjugate varies somewhat from protein to protein and can be determined by those of ordinary skill in the art without undue experimentation. The amount of additive necessary to effectively form a monomeric conjugate also varies from antibody to antibody. This amount can also be determined by one of ordinary skill in the art without undue experimentation. In U.S. Pat. Nos. 5,712,374 and 5,714,586, additions of propylene glycol in amounts ranging from 10% to 60%, preferably 10% to 40%, and most preferably about 30% by volume of the total solution, and an additive comprising at least one C₆-C₁₈ carboxylic acid or its salt, preferably caprylic acid or its salt, in amounts ranging from 20 mM to 100 mM, preferably from 40 mM to 90 mM, and most preferably about 60 mM to 90 mM were added to conjugation reactions to produce monomeric cytotoxic drug/B cell depleting agent conjugates with higher drug loading/yield and decreased aggregation. Other protein-compatible organic cosolvents other than propylene glycol, such as ethylene glycol, ethanol, DMF, DMSO, etc., could also be used. Some or all of the organic cosolvent was used to transfer the drug into the conjugation mixture.

Alternatively, in those patents, the concentration of the C₆-C₁₈ carboxylic acid or its salt could be increased to 150-300 mM and the cosolvent dropped to 1-10%. In one embodiment, the carboxylic acid was octanoic acid or its salt. In a preferred embodiment, the carboxylic acid was decanoic acid or its salt. In another preferred embodiment, the carboxylic acid was caprylic acid or its salt, which was present at a concentration of 200 mM caprylic acid together with 5% propylene glycol or ethanol.

In another alternative embodiment in those patents, t-butanol at concentrations ranging from 10% to 25%, preferably 15%, by volume of the total solution could be added to the conjugation reaction to produce monomeric cytotoxic drug/B cell depleting agent conjugates with higher drug loading/yield and decreased aggregation.

These established conjugation conditions were applied to the formation of CMA-676 (Gemtuzumab Ozogamicin), which is now commercially sold as Mylotarg™. Since introduction of this treatment for acute myeloid leukemia (AML), it has been learned through the use of ion-exchange chromatography that the calicheamicin is not distributed on the antibody in a uniform manner. Most of the calicheamicin is on approximately half of the antibody, while the other half exists in a LCF that contains only small amounts of calicheamicin. Consequently, there is a critical need to improve the methods for conjugating cytotoxic drugs such as calicheamicins to B cell depleting agents which minimize the amount of aggregation and allow for a higher uniform drug loading with a significantly improved yield of the conjugate product.

A specific example is that of the G5/44-NAc-gamma-calicheamicin DMH AcBut conjugate, which is generically shown in FIG. 17. The reduction of the amount of the LCF to <10% of the total antibody was desired for development of the conjugate, and various options for reduction of the levels of the LCF were considered. Other attributes of the immunoconjugate, such as antigen binding and cytotoxicity, must not be affected by the ultimate solution. The options considered included genetic or physical modification of the antibody, the chromatographic separation techniques, or the modification of the reaction conditions.

Reaction of the G5/44 antibody with NAc-gamma-calicheamicin DMH AcBut OSu using the old reaction conditions resulted in a product with similar physical properties (drug loading, LCF, and aggregation) as with conditions described above. However, the high level (50-60%) of LCF present after conjugation was deemed undesirable. Optimal reaction conditions were determined through statistical experimental design methodology in which key reaction variables such as temperature, pH, calicheamicin derivative input, and additive concentration, were evaluated. Analysis of these experiments demonstrated that calicheamicin input and additive concentration had the most significant effects on the level of the low conjugated fraction, LCF, and aggregate formation, while temperature and pH exerted smaller influences. In additional experiments, it was also shown that the concentrations of protein B cell depleting agent (antibody) and cosolvent (ethanol) were similarly of lesser importance (compared to calicheamicin input and additive concentration) in controlling LCF and aggregate levels. In order to reduce the LCF to <10%, the calicheamicin derivative input was increased from 3% to 8.5% (w/w) relative to the amount of antibody in the reaction. The additive was changed from octanoic acid or its salt at a concentration of 200 mM to decanoic acid or its salt at a concentration of 37.5 mM. The conjugation reaction proceeded better at slightly elevated temperature (30-35° C.) and pH (8.2-8.7). The reaction conditions incorporating these changes reduced the LCF to below 10 percent while increasing calicheamicin loading, and is hereinafter referred to as “new” process conditions. A comparison of the results obtained with the new and old process conditions is shown in Table 1.

TABLE 1
Comparison of the old and new process conditions

| | Old Process | New Process |
|---|---|---|
| Conditions/Results | Conditions | Conditions |
| Calicheamicin Input | 3.0% (w/w powder | 8.5% (w/w) |
| | weight basis) | |
| Additive Identity and | Octanoic | Decanoic acid/Sodium |
| Concentration | acid/Sodium | decanoate; 37.5 mM |
| | octanoic; 200 mM | |
| Temperature | 26° C. | 31-35° C. |
| PH | 7.8 | 8.2-8.7 |
| Calicheamicin Loading | 2.4-3.5 | 7.0-9.0 |
| (percent by weight; | | |
| by UV assay) | | |
| LOW CONJUGATED | 45-65 HPLC Area % | <10% |
| FRACTION (LCF) | | |
| (BEFORE | | |
| PURIFICATION) | | |
| Aggregation (before | ˜5% | <5% |
| purification) | | |
| Aggregation (after | ≦2% | <2% |
| purification) | | |

The increase in calicheamicin input increased the drug loading from 2.5-3.0 weight percent to 7.0-9.0 (most typically 7.5-8.5) weight percent, and resulted in no increase in protein aggregation in the reaction. Due to reduction of aggregate and LCF, the New Process Conditions resulted in a more homogeneous product. New process conditions have been reproducibly prepared by this new conjugation procedure at the multi-gram antibody scale.

In the foregoing reactions, the concentration of antibody can range from 1 to 15 mg/ml and the concentration of the calicheamicin derivative, e.g., N-Acetyl gamma-calicheamicin DMH AcBut OSu ester (used to make the conjugates shown in FIG. 17), ranges from about 4.5-11% by weight of the antibody. The cosolvent was ethanol, for which good results have been demonstrated at concentrations ranging from 6 to 11.4% (volume basis). The reactions were performed in PBS, HEPES, N-(2-Hydroxyethyl)piperazine-N′-(4-butanesulfonic acid) (HEPBS), or other compatible buffer at a pH of 8 to 9, at a temperature ranging from 30° C. to about 35° C., and for times ranging from 15 minutes to 24 hours. Those who are skilled in the art can readily determine acceptable pH ranges for other types of conjugates. For various antibodies the use of slight variations in the combinations of the aforementioned additives have been found to improve drug loading and monomeric conjugate yield, and it is understood that any particular protein B cell depleting agent may require some minor alterations in the exact conditions or choice of additives to achieve the optimum results.

Conjugate Purification and Separation

Following conjugation, the monomeric conjugates may be separated from the unconjugated reactants (such as B cell depleting agent and free cytotoxic drug/calicheamicin) and/or the aggregated form of the conjugates by conventional methods, for example, size exclusion chromatography (SEC), hydrophobic interaction chromatography (HIC), ion exchange chromatography (IEC), or chromatofocusing (CF). The purified conjugates are monomeric, and usually contain from 4 to 10% by weight cytotoxic drug/calicheamicin. In a preferred embodiment, the conjugates are purified using hydrophobic interaction chromatography (HIC). In the processes previously used for the production-scale manufacturing of cytotoxic drug/calicheamicin-antibody conjugates, the sole post-conjugation separation step employed was size exclusion chromatography (SEC). While this step is quite effective at both removing aggregated conjugate and in accomplishing buffer exchange for formulation, it is ineffective at reducing the LCF content. Consequently, the SEC-based process relies entirely on the chemistry of the conjugation reaction to control the LCF content of the final product. Another disadvantage of SEC is the limitation of the volume of conjugate reaction mixture applied to the column (typically not exceeding 5 percent of the process column bed volume). This severely limits the batch size (and therefore production capacity) that can be supported in a given production space. Finally, the SEC purification process also results in significant dilution of the conjugate solution, which places constraints on the protein concentration that can be dependably achieved in formulation.

When a cytotoxic drug has a highly hydrophobic nature, such as a calicheamicin derivative, and is used in a conjugate, hydrophobic interaction chromatography (HIC) is a preferred candidate to provide effective separation of conjugated and unconjugated antibody. HIC presents three key advantages over SEC: (1) it has the capability to efficiently reduce the LCF content as well as the aggregate; (2) the column load capacity for HIC is much higher; and (3) HIC avoids excessive dilution of the product.

A number of high-capacity HIC media suitable for production scale use, such as Butyl, Phenyl and Octyl Sepharose 4 Fast Flow (Amersham Biosciences, Piscataway, N.J.), can effectively separate unconjugated components and aggregates of the conjugate from monomeric conjugated components following the conjugation process.

Anti-Cytokine Agents

The present invention contemplates the use of anti-cytokine agents for the treatment of autoimmune diseases. In particular, the present invention provides anti-cytokine agents in combination with a B cell depleting agent or conjugate of the present invention. Preferably, an anti-cytokine is provided in combination with a cytotoxic drug/B cell depleting agent conjugates. The anti-cytokine agents are provided for administration to patients with an autoimmune condition or at risk of developing an autoimmune condition. The anti-cytokine agents of the present invention include any agent effective against a cytokine and the like. The present invention contemplates the use of any type of anti-cytokine agent, as known to persons skilled in the art, for example, soluble recombinant cytokine receptors, antibodies to cytokines, small molecules that effects the activity of cytokines, antisense oligonucleotides or combinations thereof, without limitation.

Preferably, the anti-cytokine agent of the present invention is an anti-TNF agent. Any effective anti-TNF agent is contemplated by the present invention. For example, without limitation, the anti-TNF agent may be a soluble recombinant receptor, a chimeric protein, a small molecule, an anti-TNF antibody, an antisense oligonucleotide, an anti-TNF immunoreceptor peptide, an anti-idiotype antibody, a structural analog of an anti-TNF antibody or peptide or any combination thereof.

Soluble Recombinant Receptors and Chimeric Proteins

The anti-cytokine agent may include a soluble receptor such as a TNF receptor and a TNFR-Ig. Two distinct types of TNFR are known to exist: Type I TNFR (TNFRI) and Type II TNFR (TNFRII). The mature full-length human TNFRII is a glycoprotein having a molecular weight of about 75-80 kilodaltons (kDa). The mature full-length human TNFRH is a glycoprotein having a molecular weight of about 55-60 kitodaltons (kDa). The preferred TNFRs of the present invention are soluble forms of TNFRI and TNFRII, as well as soluble TNF binding proteins.

Soluble anti-cytokine molecules include, for example, analogs or subunits of native proteins having at least 20 amino acids. Soluble TNFR, for example, exhibits at least some biological activity in common with TNFRI, TNFRII or TNF binding proteins. Soluble TNFR constructs are devoid of a transmembrane region (and are secreted from the cell) but retain the ability to bind TNF. Various bioequivalent protein and amino acid analogs have an amino acid sequence corresponding to all or part of the extracellular region of a native receptor.

Equivalent soluble TNFRs include polypeptides which vary from these sequences by one or more substitutions, deletions, or additions, and which retain the ability to bind TNF or inhibit TNF signal transduction activity via cell surface bound TNF receptor proteins. Analogous deletions may be made to muTNFR. Inhibition of TNF signal transduction activity can be determined by transfecting cells with recombinant TNFR DNAs to obtain recombinant receptor expression. The cells are then contacted with TNF and the resulting metabolic effects examined. If an effect results which is attributable to the action of the ligand, then the recombinant receptor has signal transduction activity. Exemplary procedures for determining whether a polypeptide has signal transduction activity are disclosed by Idzerda et. al., J. Exp. Med. 171:861 (1990); Curtis et al., Proc. Natl. Acad. Sci. U.S.A. 86:3045 (1989); Prywes et al. EMBO J. 5:2179 (1986) and Chou et al., J. Biol. Chem. 262:1842 (1987). Alternatively, primary cells or cell lines which express an endogenous TNF receptor and have a detectable biological response to “INF could also be utilized.

The nomenclature for TNFR analogs as used herein follows the convention of naming the protein (e.g., TNFR) preceded by either hu (for human) or mu (for murine) and followed by a Δ (to designate a deletion) and the number of the C-terminal amino acid. For example, huTNFRΔ 235 refers to human TNFR having Asp235 as the C-terminal amino acid. In the absence of any human or murine species designation, TNFR refers generically to mammalian TNFR. Similarly, in the absence of any specific designation for deletion mutants, the term TNFR means all forms of TNFR, including routants and analogs which possess TNFR biological activity.

In a preferred embodiment, the TNFR-Ig is TNFR:Fc, which may be administered in the form of a pharmaceutically acceptable composition as described herein. The diseases described herein may be treated by administering TNFR:Fc one or more times per week by subcutaneous injection, although other routes of administration may be used if desired. In one exemplary regimen for treating adult human patients, 25 mg of TNFR:Fc is administered by subcutaneous injection two times per week or three times per week for one or more weeks, and preferably for four or more weeks. Alternatively, a dose of 5-12 mg/m.sup.2 or a flat dose of 50 mg is injected subcutaneously one time or two times per week for one or more weeks. In other embodiments, psoriasis is treated with TNFR:Fc in a sustained-release form, such as TNFR:Fc that is encapsulated in a biocompatible polymer, TNFR:Fc that is admixed with a biocompatible polymer (such as topically applied hydrogels), and TNFR:Fc that is encased in a semi-permeable implant.

Various other medicaments may also be administered concurrently with compositions comprising anti-cytokine agents. Such medicaments include: NSAIDs; DMARDs; analgesics; topical steroids; systemic steroids (e.g., prednisone); cytokine; antagonists of inflammatory cytokines; antibodies against T cell surface proteins; oral retinoids; salicylic acid; and hydroxyurea. Suitable analgesics for such combinations include: acetaminophen, codeine, propoxphene napsylate, oxycodone hydrochloride, hydrocodone bitartrate and tramadol. DMARDs suitable for such combinations include: azathioprine, cyclophosphamide, cyclosporine, hydroxychloroquine sulfate, methotrexate, leflunomide, minocycline, penicillamine, sulfasalazine, oral gold, gold sodium thiomalate and aurothioglucose. In addition, the anti-cytokine agent may be administered in combination with antimalarials or colchicine. NSAIDs suitable for the subject combination treatments include: salicylic acid (aspirin) and salicylate derivatives; ibuprofen; indomethacin; celecoxib (CELEBREX); rofecoxib (VIOXX); ketorolac; nambumetone; piroxicam; naproxen; oxaprozin; sulindac; ketoprofen; diclofenac; and other COX-1 and COX-2 inhibitors, propionic acid derivatives, acetic acid derivatives, carboxylic acid derivatives, carboxylic acid derivatives, butyric acid derivatives, oxicams, pyrazoles and pyrazolones, including newly developed anti-inflammatories.

If an antagonist against an inflammatory cytokine is administered concurrently with TNFR:Fc, suitable targets for such antagonists include TGF-beta, IL-6 and IL-8.

In addition, the anti-cytokine may be used in combination with topical steroids, systemic steroids, antagonists of inflammatory cytokines, antibodies against T cell surface proteins, methotrexate, cyclosporine, hydroxyurea and sulfasalazine.

An appropriate dose of the anti-cytokine agent may be determined according to the animal's body weight. For example, a dose of 0.2-1 mg/kg may be used. Alternatively, the dose is determined according to the animal's surface area, an exemplary dose ranging from 0.1-20 mg/m.sup.2, or more preferably, from 5-12 mg/m.sup.2. For small animals, such as dogs or cats, a suitable dose is 0.4 mg/kg. In a preferred embodiment, TNFR:Fc (preferably constructed from genes derived from the same species as the patient) or another soluble TNFR mimic is administered by injection or other suitable route one or more times per week until the animal's condition is improved, or it may be administered indefinitely.

Anti-cytokine agents such as TNF antagonist proteins may be administered to a mammal, preferably a human, for the purpose treating autoimmune diseases. Because of the primary roles, interlukens, for example IL-1, IL-2 and IL-6, play in the production of TNF, combination therapy using TNFR in combination with IL-1R and/or IL-2R is contemplated. In the treatment of humans, soluble human TNFR is preferred. Either Type I IL-1R or Type II IL-1R, or a combination thereof, may be used in accordance with the present invention. Other types of TNF binding proteins may be similarly used.

The subject methods may involve administering to the patient a soluble TNF antagonist that is capable of reducing the effective amount of endogenous biologically active TNF, such as by reducing the amount of TNF produced, or by preventing the binding of TNF to its cell surface receptor. Antagonists capable of inhibiting this binding include receptor-binding peptide fragments of TNF, antisense oligonucleotides or ribozymes that inhibit TNF production, antibodies directed against TNF, and recombinant proteins comprising all or portions or receptors for TNF or modified variants thereof, including genetically-modified muteins, multimeric forms and sustained-release formulations.

Preferred embodiments of the invention utilize soluble TNFRs as the anti-cytokine agent. Soluble forms of TNFrs may include monomers, fusion proteins (also called “chimeric proteins), dimers, trimers or higher order multimers. In certain embodiments of the invention, the soluble TNFR derivative is one that mimics the 75 kDa TNFR or the 55 kDa TNFR and that binds to TNF in the patient's body. The soluble TNFR mimics may be derived from TNFRs p55 or p75 or fragments thereof. TNFRs other than p55 and p75 also are useful in the present invention, such as for example the TNFR that is described in WO 99/04001. Soluble TNFR molecules used to construct TNFR mimics include, for example, analogs or fragments of native TNFRs having at least 20 amino acids, that lack the transmembrane region of the native TNFR, and that are capable of binding TNF. Antagonists derived from TNFRs compete for TNF with the receptors on the cell surface, thus inhibiting TNF from binding to cells, thereby preventing it from manifesting its biological activities. Binding of soluble TNFRs to TNF or LT can be assayed using ELISA or any other convenient assay.

The soluble TNFR polypeptides or fragments of the invention may be fused with a second polypeptide to form a chimeric protein. The second polypeptide may promote the spontaneous formation by the chimeric protein of a dimer, trimer or higher order multimer that is capable of binding a TNF or a LT molecule and preventing it from binding to cell-bound receptors. Chimeric proteins used as antagonists include, for example, molecules derived from the constant region of an antibody molecule and the extracellular portion of a TNFR. Such molecules are referred to herein as TNFR-Ig fusion proteins, A preferred TNFR-Ig fusion protein suitable for treating diseases in humans and other mammals is recombinant TNFR:Fc, also known as etanercept and available from Immunex Corporation, a subsidiary of Amgen, under the trade name ENBREL. Because the p75 receptor protein of etanercept binds not only to TNF-α but also to the inflammatory cytokine LT-α, etanercept can act as a competitive inhibitor not only of TNF-α, but also of LT-α. This is in contrast to antibodies directed against TNF-α which cannot inhibit LT-α.

Anti-cytokines of the present invention include a compound that comprises the extracellular portion of the 55 kDa TNFR fused to the Fc portion of IgG, as well as compositions and combinations containing such a molecule. Encompassed also are soluble TNFRs derived from the extracellular regions of TNF-α receptor molecules other than the p55 and p75 TNFRs, such as for example the TNFR described in WO 99/04001, incorporated by reference in its entirety, including TNFR-Ig's derived from this TNFR. Other suitable TNF-α inhibitors include the humanized anti-TNF-α, antibody D2E7 (Knoll Pharmaceutical/BASF AG).

Sustained-release forms of anti-cytokine agents are contemplated by the present invention, including sustained-release forms of TNFR:Fc. Sustained-release forms suitable for use in the disclosed methods include, but are not limited to, agents that are encapsulated in a slowly-dissolving biocompatible polymer (such as the alginate microparticles described in U.S. Pat. No. 6,036,978 or the polyethylene-vinyl acetate and poly(lactic-glucolic acid) compositions described in U.S. Pat. No. 6,083,534), admixed with such a polymer (including topically applied hydrogels), and or encased in a biocompatible semi-permeable implant. In addition, a soluble TNFR type 1 or type II for use in the herein described therapies may be conjugated with polyethylene glycol (pegylated) to prolong its serum half-life or to enhance protein delivery.

Small Molecules

Other suitable anti-cytokine agents of the present invention include small molecules such as thalidomide or thalidomide analogs, pentoxifylline, or matrix metalloproteinase (MMP) inhibitors or other small molecules. Suitable MMP inhibitors include, for example, those described in U.S. Pat. Nos. 5,883,131, 5,863,949 and 5,861,510 as well as the mercapto alkyl peptidyl compounds described in U.S. Pat. No. 5,872,146, each of which is incorporated by reference in its entirety. Small molecules capable of reducing TNF production include, for example, the molecules described in U.S. Pat. Nos. 5,508,300, 5,596,013 and 5,563,143, any of which can be administered in combination with Anti-TNF agents such as soluble TNFRs or antibodies against TNF. Additional small molecules useful for treating the TNF-mediated diseases described herein include the MMP inhibitors that are described in U.S. Pat. No. 5,747,514, U.S. Pat. No. 5,691,382, as well as the hydroxamic acid derivatives described in U.S. Pat. No. 5,821,262. The diseases described herein also may be treated with small molecules that inhibit phosphodiesterase IV and TNF production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291 422 A). Also useful are thiazole derivatives that suppress TNF and IFNδ (WO 99/15524), as well as xanthine derivatives that suppress TNF and other proinflammatory cytokines (see. for example, U.S. Pat. No. 5,118,500, U.S. Pat. No. 5,096,906 and U.S. Pat. No. 5,196,430). Additional small molecules suitable as anti-cytokine agents include those disclosed in U.S. Pat. No. 5,547,979. Each foregoing reference is incorporated herein in its entirety.

Antisense Oligonucleotides

Also included among the anti-cytokine agents, such as anti-TNF agents, of the present invention are antisense oligonucleotides that act to directly block the translation of mRNA by hybridizing to targeted mRNA and preventing polypeptide translation. Antisense oligonucleotides are suitable for the present invention, either alone or in combination with other anti-cytokine agents or in combination with other agents. For example, antisense molecules of the invention may interfere with the translation of TNF, a TNF receptor, or an enzyme in the metabolic pathways for the synthesis of TNF. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). The ability to hybridize will depend on both the degree of complementarity and the length of the antisense nucleic acid. Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′-non-translated, non-coding regions of the targeted transcript can be used. Oligonucleotides complementary to the 5′ untranslated region of the mRNA should include the complement of the AUG start codon.

Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at feast 50 nucleotides. Most preferably, they will contain 18-21 nucleotides.

The backbone of antisense oligonucleotides may be chemically modified to prolong the hall-life of the oligonucleotide in the body. Suitable modifications for this purpose are known in the art, such as those disclosed, tot example, in U.S. Pat. No. 114,517, which describes the use for this purpose of phosphorothioates, phosphorodithioates, phospholriesters, aminoalkylphosphotriesters, methyl and other alkyl phosphonates, various phosphonates, phosphinates, and phosphoramidates and so on.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc, Natl. Acad. Sci. U.S.A. 86:6553-6556; Lemaitre et. al., 1987, Proc. Natl. Acad. Sci. 84:648-652; PCT Publication No. WO88/09810, published Dec. 15, 1988), or hybridization-triggered cleavage agents or intercalating agents. (See, e.g., Zon, 1988, Pharm. Res. 5:539-549). The antisense molecules should be delivered to cells which express the targeted transcript.

Antisense oligonucleotides can be administered parenterally, including by intravenous or subcutaneous injection, or they can be incorporated into formulations suitable for oral administration. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the targeted mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vestors can be constructed by recombinant DNA technology methods standard in the art. Vectors can be plasmid, viral, or others known in the art, used for replication and expression in mammalian cells. Antisense oligonucleotides for suitable for treating diseases associated with elevated TNF include, for example, the anti-TNF oligonucleotides described in U.S. Pat. No. 6,080,580, incorporated herein by reference in its entirety.

Ribozyme molecules designed to catalytically cleave mRNA transcripts can also be used to prevent the translation of mRNAs encoding TNF, TNF receptors, or enzymes involved in synthesis of TNF or TNFRs (see. e.g., PCT WO90/11,364; U.S. Pat. No. 5,824,519). Ribozymes useful for this purpose include hammerhead ribozymes (Haseloff and Gerlach, 1988, Nature, 334:585-591), RNA endoribonucleases (hereinafter “Cech-type ribozymes”) such as the one that occurs naturally in Tetrahymena thermophila (known as the IVS, or L-19 IVS RNA) (see, for example, WO 88/04300; Been and Cech, 1986, Cell, 47:207-216). Ribozymes can be composed of modified oligonucleotides (e.g. for improved stability, targeting, etc.) and should be delivered to cells which express the target peptide in vivo. A preferred method of delivery involves using a DNA construct encoding the ribozyme under the control of a strong constitutive pol III or pol II promoter, so that transfected cells will produce sufficient quantities of the ribozyme to destroy endogenous target mRNA, thereby inhibiting its translation.

Alternatively, expression of genes involved in TNF or TNFR production can be reduced by targeting deoxyribonucleotide sequences complementary to the regulatory region of the target gene (i.e., the target gene promoter and/or enhancers) to form triple helical structures that prevent transcription of the target gene. (See, for example, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Antisense RNA and DNA, ribozyme, and triple helix molecules of the invention may be prepared by any method known in the art for the synthesis of DNA and RNA molecules, including, for example, solid phase phosphoramidite chemical synthesis. Oligonucleotides can be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209, and methylphosphonate oligonucleotides can be prepared as described by Sarin et al., 1988, Proc. Natl. Acad. Sci. U.S.A. 85:7448-7451. Alternatively, RNA molecules may generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capeechi, 1987, Cell 51, 503-512; Thompson, et al., 1989, Cell 5, 313-321). For example, a mutant, nonfunctional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok A, Tabara H, and Mello C C, 2000, Science 287 (5462): 2494-2497), or the introduction of transgenes (Dernburg et al., 2000, Genes Dev. 14 (13): 1578-1583) are used to inhibit the expression of specific target genes. This approach can be adapted for use in humans provided the recombinant DNA constructs are directly administered or targeted to the required site in vivo using appropriate vectors such as viral vectors.

Anti-Cytokine Antibodies

The anti-cytokine agents suitable for the present invention include polyclonal antibodies, monoclonal antibodies (mAbs), chimeric antibodies, anti-idiotypic (anti-Id) antibodies to antibodies that can be labeled in soluble or bound form, as well as fragments, regions or derivatives thereon, provided by any known technique, such as, but not limited to enzymatic cleavage, peptide synthesis or recombinant techniques are contemplated by the present invention. For example, anti-TNF antibodies of the present invention include those capable of binding portions of TNF that inhibit the binding of TNF to TNF receptors.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. mAbs may be obtained by methods known to those skilled in the art. See, for example Kohler and Milstein. Nature 256:495-497 (1975); U.S. Pat. No. 4,376,110; Ausubel et al., eds., CURRENT PROTOCOLS IN MOLECULAR BIOLOGY, Greene Publishing Assoc. and Wiley Interscience, N.Y., (1987, 1992); and Harlow and Lane ANTIBODIES: A LABORATORY MANUAL Cold Spring Harbor Laboratory (1988); Colligan et al., eds., Current Protocols in Immunology, Greene Publishing Assoc. and Wiley Interscience, N.Y., (1992, 1993), the contents of which references are incorporated entirely herein by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of mAbs in vivo or in situ makes this the presently preferred method or production.

Chimeric antibodies are molecules different portions of which are derived from different animal species, such as those having variable region derived from a murine mAb and a human immunoglobulin constant region, which are primarily used to reduce immunogenicity in application and to increase yields in production, for example, where murine mAbs have higher yields from hybridomas but higher immunogenicity in humans, such that human murine chimeric mAbs are used. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (194), Boulianne et al., Nature 312: 643-646 (1984); Cabilly et al., European Patent Application 125023 (published Nov. 14, 1984); Neuberger et al., Nature 314:268-270 (1985); Taniguchi et al., European Patent Application 17/496 (published Feb. 19, 1985); Morrison et al., European Patent Application 173494 (published Mar. 5, 1986); Neuberger et al., PCT Application WO 86/01533, (published Mar. 13, 1986); Kudo et al., European Patent Application 184187 (published Jun. 11, 1986); Morrison et al., European Patent Application (published Mar. 5, 1986); Sahagan et al., J. Immunol. 137:1066-1074 (1986): Robinson et al., International Patent Publication #PCT/US86/02269 (published May 7, 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane ANTIBODIES: A LABORATORY MANUAL Cold Spring Harbor Laboratory (1988)).

Polyclonal murine antibodies to TNF are disclosed by Cerami et al (EPO Patent Publication 0212489, Mar. 4, 1987).

Rubin et al. (EPO Patent Publication 0218868, Apr. 22, 1987) discloses murine monoclonal antibodies to human TNF, the hybridomas secreting such antibodies, methods of producing such murine antibodies, and the use of such murine antibodies in immunoassay of TNF.

Yone et al. (EPO Patent Publication 0288088, Oct. 26, 1988) discloses anti-TNF murine antibodies, including mAbs, and their utility in immunoassay diagnosis of pathologies, in particular Kawasaki's pathology and bacterial infection.

Other investigators have described rodent or routine mAbs specific for recombinant human TNF which had neutralizing activity in vitro (Liang, et al., (Biochem. Biophys. Res. Comm. 137:847-854 (1986); Meager, et al., Hybridoma 6:305-311 (1987); Fendly et al., Hybridoma 6:359-369 (1987); Bringman, et al. Hybridoma 6:489-507 (1987); Hirai, et al., J. Immunol. Meth. 96:57-62. (1987) Moiler, et al., (Cytokine 2:162-169 (1990)).

Neutralizing antisera or mAbs to TNF have been shown in mammals other than man to abrogate adverse physiological changes and prevent death after lethal challenge in experimental endotoxemia and bacteremia. This effect has been demonstrated, e.g., in rodent lethality assays and in primate pathology model systems (Mathison, et al., J. Clin. Invest. 81:1925-1937 (1988); Beutler, et al., Science 229:869-871 (1985); Tracey, et al., Nature 330:662-664 (1987); Shimamoto, et al., Immunol. Lett. 17:311-318 (1988); Silva, et al., J. Infect. Dis. 162:421-427 (1990); Opal et al., J. Infect. Dis. 161:1148-1152 (1990); Hinshaw, et al., Circ. Shock 30:279-292 (1990)).

An anti-idiotypic (anti-id) antibody is an antibody which recognizes unique determinants generally associated with the antigen-binding site of an antibody. An Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). See for example, U.S. Pat. No. 4,699,880, which is herein entirely incorporated by reference.

The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb it is possible to identify other clones expressing antibodies of identical specificity.

Anti-TNF antibodies of the present invention can include at least one of a heavy chain constant region (H_{c}) a heavy chain variable region (Hᵥ), a light chain variable region (Lᵥ) and a light chain constant regions (L_{c}), wherein a polyclonal Ab, monoclonal Ab, fragment and/or regions thereof include at least one heavy chain variable region (Hᵥ) or light chain variable region (Lᵥ) which binds a portion of a TNF and inhibits and/or neutralizes at least one TNF biological activity.

An antigen is a molecule or a potion of a molecule capable of being bound by an antibody which is additionally capable of inducing an animal to produce antibody capable of binding to an epitope of that antigen. An antigen can have one or more than one epitope.

The specific reaction referred to above is meant to indicate that the antigen will react, in a highly selective manner, with its corresponding antibody and not with the multitude of other antibodies which can be evoked by other antigens. Preferred, antigens that bind antibodies, fragments and regions of anti-TNF antibodies of the present invention include at least 5 amino acids comprising at least one of amino acids residues 87-108 or both residues 59-80 and 8-108 of hTNF-α (SEQ ID NO:52). Preferred antigens that bind antibodies, fragments and regions of anti-TNF antibodies of the present invention do not include amino acids of amino acids 11-13, 37-42, 49-57 or 155-157 of hTNF-α (SEQ ID NO: 52).

The epitope is that portion of any molecule capable of being recognized by and bound by an antibody at one or more of the Ab's antigen binding region. Epitopes usually consist of chemically active surface groupings of molecules such as amino acids or sugar side chains and have specific three dimensional structural characteristics as well as specific charge characteristics. By “inhibiting and/or neutralizing epitope” is intended an epitope, which, when bound by an antibody, results in loss of biological activity of the molecule or organism containing the epitope, in vivo, in vitro or in site, more preferably in vivo, including, for example, binding of TNF to a TNF receptor. For instance, those disclosed in U.S. Pat. No. 6,277,969 which is incorporated herein by reference in its entirety.

Murine and chimeric antibodies, fragments and regions of the present invention comprise individual heavy (H) and/or light (L) immunoglobulin chains. A chimeric H chain comprises an antigen binding region derived from the H chain of a non-human antibody specific for TNF, which is linked to at least a portion of a human H chain C region (C_{H}), such as CH₁ or CH₂.

A chimeric L chain according to the present invention, comprises an antigen binding region derived from the L chain of a ram-human antibody specific for TNF linked to at least a portion of a human L chain C region (C_{L}).

Antibodies, fragments or derivatives having chimeric H chains and L chains of the same or different variable region binding specificity, can also be prepared by appropriate association of the individual polypeptide chains, according to known method steps, e.g., according to Ausubel infra, Harlow infra, and Colligan infra.

Anti-Cytokine Immunoreceptor Peptides

Immunoreceptor peptides of this invention can bind to cytokines, such as TNF-α and/or TNF-β. The immunoreceptor comprises covalently attached to at least a portion of the receptor at least one immunoglobulin heavy or light chain. In certain preferred embodiments, the heavy chain constant region comprises at least a portion of CH₁. Specifically, where a light chain is included with an immunoreceptor peptide, the heavy chain must include the area of CH₁ responsible for binding a light chain constant region.

An immunoreceptor peptide of the present invention can preferably comprise at least one heavy chain constant region and in certain embodiments, at least one light chain constant region, with a receptor molecule covalently attached to at least one of the immunoglobulin chains. Light chain or heavy chain variable regions are included in certain embodiments. Since the receptor molecule can be linked within the interior of an immunoglobulin chain, a single chain can have a variable region and a fusion to a receptor molecule.

The portion of the TNF receptor linked to the immunoglobulin molecule is capable of binding TNF-α and/or TNF-β. Since the extracellular region of the TNF receptor binds TNF, the portion attached to the immunoglobulin molecule of the immunoreceptor consists of at least a portion of the extracellular region of the TNF receptor.

The immunoglobulin gene can be from any vertebrate source, such as murine, but preferably, it encodes immunoglobulin having a substantial humor of sequences that are of the same origin as the eventual recipient of the immunoreceptor peptide. For example, if a human is treated with a molecule of the invention, preferably the immunoglobulin is of human origin.

TNF receptor constructs for lining to the heavy chain can be synthesized, for example, using DNA encoding amino acids present in the cellular domain of the receptor. Putative receptor binding loci of hTNF have been presented by Eck and Sprange, J. Biol. Chem. 264(29), 17595-17605 (1989), who identified the receptor binding loci of TNF-α as consisting of amino acids 11-13, 37-42, 49-57 and 155-157. PCT application WO91/02078 (priority date of Aug. 7, 1989) discloses TNF ligands which can bind to monoclonal antibodies having the following epitopes of at least one of 1-20, 56-77, and 108-127; at least two of 1-20, 56-77, 108-127 and 138-149; all of 1-18, 58-65, 115-125 and 138-149; all of 1-18, and 108-128; all of 56-79, 110-127 and 135- or 136-155; all of 1-30 and 117-128 and 141-153; all of 1-26, 117-128 and 141-153; all of 22-40, 49-96 or -97, 11-127 and 136-153; all of 12-22, 36-45, 96-105 and 132-157; all of both of 1-20 and 76-90; all of 22-40, 69-97, 105-128 and 135-155; all of 22-31 and 146-157; all of 22-40 and 49-98; at least one of 22-40, 9-98 and 69-97, both of 22-40 and 70-87. Thus, one skilled in the art once armed with the present disclosure, would be able to create TNF receptor fusion proteins using portions of the receptor that are known to bind TNF.

Advantages of using an immunoglobulin fusion protein (immunoreceptor peptide) of the present invention include one or more of (1) possible increased avidity for multivalent ligands due to the resulting bivalency of dimeric fusion proteins, (2) longer serum half-life, (3) the ability to activate effector cells via the Fc domain, (4) ease of purification (for example, by protein A chromatography), (5) affinity for TNF-α and TNF-β and (6) the ability to block TNF-α or TNF-β cytotoxicity.

While this generally permits secretion of the fusion protein in the absence of an Ig light chain, a major embodiment of the present invention provides for the inclusion of the CH₁ domain, which can confer advantages such as (1) increased distance and/or flexibility between two receptor molecules resulting in greater affinity for TNF, (2) the ability to create a heavy chain fusion protein and a light chain fusion protein that would assemble with each other and dimerize to form a tetravalent (double fusion) receptor molecule, and (3) a tetravalent fusion protein can have increased affinity and/or neutralizing capability for TNF compared to a bivalent (single fusion) molecule.

Anti-Idiotype ABS

In addition to monoclonal or chimeric anti-cytokine antibodies, such as anti-TNF antibodies, the present invention also contemplates an anti-idiotypic (anti-Id) antibody specific for the anti-cytokine, e.g., anti-TNF antibody, of the invention. An anti-id antibody is an antibody which recognizes unique determinants generally associated with the antigen-binding region of another antibody. For example, the antibody specific for TNF is termed the idiotypic or Id antibody. The anti-Id can be prepared by immunizing an animal of the same species and genetic type (e.g. mouse strain) as the source of the Id antibody with the Id antibody or the antigen-binding region thereof. The immunized animal will recognize and respond to the idiotypic determinants of the immunizing antibody and produce an anti-Id antibody. The anti-Id antibody can also be used as an “immunogen” to induce an immune response in yet another animal, producing a so-called anti-anti-Id antibody. The anti-anti-Id can be epitopically identical to the original antibody which induced the anti-Id. Thus, by using antibodies to the idiotypic determinants of a mAb, it is possible to identify other clones expressing antibodies of identical specificity.

Accordingly, mAbs generated against cytokines such as TNF according to the present invention can be used to induce anti-Id antibodies in suitable animals, such as BALB/c mice. Spleen cells from such immunized mice can be used to produce anti-id hybridomas secreting anti-Id mAbs. Further, the anti-Id mAbs can be coupled to a B cell depleting agent such as keyhole limit hemocyanin (KLH) and used to immunize additional BALB/c mice. Sera from these mice will contain anti-anti-Id antibodies that have the binding properties of the original mAb specific for a TNF epitope.

Accordingly, any suitable cytokine neutralizing compound can be used in methods according to the present invention. For example, TNF neutralizing compound can be selected from the group consisting of antibodies or portions thereof specific to neutralizing epitopes of TNF, p55 receptors, p75 receptors, or complexes thereof, portions of TNF receptors which bind TNF, peptides which bind TNF, peptido mimetic drugs which bind TNF and any organo mimetic drugs that block TNF.

Such TNF neutralizing compounds can be determined by routine experimentation based on the teachings and guidance presented herein, by those skilled in the relevant arts.

Structural Analogs of Anti-TNF Antibodies and Anti-TNF Peptides

Structural analogs of anti-TNF Abs and peptides of the present invention are provided by known method steps based on the teaching and guidance presented herein.

Knowledge of the three-dimensional structures of proteins is crucial in understanding how they function. The three-dimensional structures of more than 400 proteins are currently available in protein structure databases (in contrast to around 15,000 known protein sequences in sequence databases). Analysis of these structures shows that they fall into recognizable classes of motifs. It is thus possible to model a three-dimensional structure of a protein based on the proteins homology to a related protein of known structure. Many examples are known where two proteins that have relatively low sequence homology, can have very similar three dimensional structures or motifs.

In recent years it has become possible to determine the three dimensional structures of proteins of up to about 15 kDa by nuclear magnetic resonance (NMR). The technique only requires a concentrated solution of pure protein. No crystals or isomorphous derivatives are needed. The structures of a number proteins have been determined by this method. The details of NMR structure determination are well-known in the art. (See, e.g., Wuthrich., NMR of Proteins and Nucleic Acids, Wiley, New York, 1986; Wuthrich, K. Science 243:45-50 (1989); Clore et al. Crit, Rev. Bioch, Molec. Biol. 24:479-564 (1989); Cooke et al., Bioassays 8:52-56 (1988)).

In applying this approach, a variety of ¹H NMR 2D data sets are collected for anti-TNF Abs and/or anti-TNF peptides of the present invention. These are of two main types. One type, COSY (Conetated Spectroscopy) identifies proton resonances that are linked by chemical bonds. These spectra provide information on protons that are linked by three or less covalent bonds. NOESY (nuclear Overhauser enhancement spectroscopy) identifies protons which are close in space (less than 0.5 nm). Following assignment of the complete spin system, the secondary structure is defined by NOESY. Cross peaks (nuclear Overhauser effects or NOE's) are found between residues that are adjacent in the primary sequence of the peptide and can be seen for protons less than 0.5 nm apart. The data gathered from sequential NOE's combined with amide proton coupling constants and NOE's from non-adjacent amino acids, that are adjacent to the secondary structure, are used to characterize the secondary structure of the polypeptides. Aside from predicting secondary structure, NOE's indicate the distance that protons are in space in both the primary amino acid sequence and the secondary structures. Tertiary structure predictions are determined, after all the data are considered, by a “best fit” extrapolation.

Types of amino acid are first identified using through-bond connectivities. The second step is to assign specific amino acids using through-space connectivities to neighboring residues, together with the known amino acid sequence. Structural information is then tabulated and is of three main kinds: The NOE identifies pairs of protons which are close in space, coupling constants give information on dihedral angles and slowly exchanging amide protons give information on the position of hydrogen bonds. The restraints are used to compute the structure using a distance geometry type of calculation followed by refinement using restrained molecular dynamics. The output of these computer programs is a family of structures which are compatible with the experimental data (i.e. the set of pairwise <0.5 nm distance restraints). The better that the structure is defined by the data, the better the family of structures can be superimposed, (i.e., the better the resolution of the structure). In the better defined structures using NMR, the position of much of backbone (i.e. the amide. C-α and carbonyl atoms) and the side chains of those amino acids that lie buried in the core of the molecule can be defined as clearly as in structures obtained by crystallography. The side chains of amino acid residues exposed on the surface are frequently less well defined, however. This probably reflects the fact that these surface residues are more mobile and can have no fixed position. (In a crystal structure this might be seen as diffuse electron density).

Thus, according to the present invention, use of NMR spectroscopic data is combined with computer modeling to arrive structural analogs of at least portions of anti-TNF Abs and peptides based on a structural understanding of the topography. Using this information, one of ordinary skill in the art will know how to achieve structural analogs of anti-TNF Abs and/or peptides, such as by rationally-based amino acid substitutions allowing the production of peptides in which the TNF binding affinity is modulated in accordance with the requirements of the expected therapeutic or diagnostic use of the molecule, preferably, the achievement of greater specificity for TNF binding.

Alternatively, compounds having the structural and chemical features suitable as anti-TNF therapeutics and diagnostics provide structural analogs with selective TNF affinity. Molecular modeling studies of TNF binding compounds, such as TNF receptors, anti-TNF antibodies, or other TNF binding molecules, using a program such as MACROMODEL, INSIGHT, and DISCOVER, provide such spatial requirements and orientation of the anti-TNF Abs and/or peptides according to the present invention. Such structural analogs of the present invention thus provide selective qualitative and quantitative anti-TNF activity in vitro, in situ and/or in vivo.

Additional Active Agents

The compositions and method of the invention may include additional active agents. The additional agents may serve, for example, as (1) adjuvants to enhance the effectiveness of the cytotoxic drug, B cell depleting agent, conjugates of same, and/or anti-cytokine agents, (2) additional actives effective against autoimmune conditions, and/or (3) actives against other conditions that the patient is suffering, including conditions that may aggravate the autoimmune condition.

The present invention contemplates agents, such as recombinant forms of a naturally occurring human protein that regulates IL-1, monoclonal antibodies that block the action of IL-1, human monoclonal antibodies directed against IL-15, small molecules that inhibits p38 MAP kinase, antagonists that reduce the production of abnormally functioning B cells, ribonucleases and combinations thereof. Such agents are described, for example, in U.S. Pat. Nos. 5,075,222 and 6,599,873; U.S. Patent Application Nos. 2002/0077294, 2002/0009454, 2003/0072756, 2003/0236193, 2004/0044001, 2004/0097712, 2003/0138421, 2004/0039029, and 2004/0044044; and published international applications WO 00/40716 and WO 01/060397, which are herein incorporated by reference in their entirety.

The present invention also contemplates the use of the conjugates and/or anti-cytokine agents of the present invention in combination with anti-viral agents, anti-bacterial agents, anti-fungal agents, anti-osteoporotic agents, immunogenic compounds, skin/sun protective agents, any agents that may treat conditions believed to aggravate autoimmune conditions or any agents that are believed to directly or indirectly aggravate autoimmune conditions.

Anti-Viral Agents

The present invention contemplates the use of anti-viral agents in combination therapies. Preferably, the anti-viral compound is an inhibitor of viral RNA-dependent RNA polymerase, an inhibitor of a virus-encoded protease that effects processing of a viral RNA-dependent RNA polymerase, an inhibitor of budding or release from infected cells, inhibitor of coronavirus budding or release from infected cells, such as one that effects the activity of hemagglutinin-esterase, an inhibitor of virus binding to a specific cell surface receptor (e.g., an inhibitor of the binding of hAPN to HCoV-229E), an inhibitor of receptor-induced conformational changes in virus spike glycoprotein that are associated with virus entry and combinations thereof.

Anti-viral compounds include nucleoside/nucleotide reverse transcriptase inhibitors (NRTIs), non-nucleoside reverse transcriptase inhibitors (NNRTIs), and/or protease inhibitors (P is), fusion inhibitors/gp41 binders, fusion inhibitors/chemokine receptor antagonists, CCR2B, CCR3, and CCR6 antagonists, chemokine receptor agonists may also inhibit fusion, integrase inhibitors, hydroxyurea-like compounds.

Other antiretroviral agents include inhibitors of viral integrase, inhibitors of viral genome nuclear translocation such as arylene bis(methylketone) compounds: inhibitors of HIV entry, soluble complexes of RANTES and glycosaminoglycans (GAG), and AMD-3100; nucleocapsid zinc finger inhibitors such as dithiane compounds: targets of HIV Tat and Rev; mid pharmacoenhancers.

According to an embodiment, the compositions of the invention may comprise other antiretroviral compounds including lymphokines.

In other embodiments, compositions of the invention additionally comprise anti-opportunistic infection agents.

Antibacterial Agents

In a further embodiment, compositions of the invention comprise an antibiotic agent. Antibiotic agents that may be administered include, but are not limited to, amoxicillin, beta-lactamases, aminoglycosides, betalactam (glycopeptide), betalactamases, Clindamycin, chloramphenicol, cephalosporins, ciprofloxacin, erythromycin, fluoroquinolones, macrolides, metronidazole, penicillins, quinolones, rapamycin, rifampin, streptomycin, sultonamide, tetracyclines, trimtethoprim, trimethoprim-sulfamethoxazole, and vancomycin.

Immunosuppressive Agents

The present invention contemplates the use of immunosuppressive agents. Immunosuppressive agents that may be administered include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells. Other immunosuppressive agents that may be administered in combination with the Therapeutics of the invention include, but are not limited to, prednisolone, methotrexate, thalidomide, methoxsalen, rapamycin, leflunomide, mizoribine (BREDNIN), brequinar, deoxyspergualin, and azaspirane (SKF 105685), ORTHOCLONE OKT 3 (muromonab-CD3), SANDIMMUNE, NEORAL, SANGDYA (cyclosporine), PROGRAF (FK506, tacrolimus), CELLCEPT (mycophenolate motefil, of which the active metabolite is mycophenolic acid), IMURAN (azathioprine), glucocorticosteroids, adrenocortical steroids such as DELTASONE (prednisone) and HYDELTRASOL (prednisolone), FOLEX and MEXATE (methotrxate), OXSORALEN-ULTRA (methoxsalen) and RAPAMUNE (sirolimus). In a specific embodiment, immunosuppressants may be used to prevent rejection of organ or bone marrow transplantation.

Immune Globulin

The compositions of the invention may comprise intravenous immune globulin preparations. Intravenous immune globulin preparations that may be administered include, but are not limited to, GAMMAR, IVEEGAM, SANDOGLOBULIN, GAMMAGARD S/D, ATGAM, (antithymocyte glubulin), and GAMIMUNE. In a specific embodiment, therapeutics of the invention are administered in combination with intravenous immune globulin preparations in transplantation therapy (e.g., bone marrow transplant).

Anti-Inflammatory Agents

In certain embodiments, the compositions of the invention comprise an anti-inflammatory agent. Anti-inflammatory agents that may be administered include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Oligonucleotides

These antisense molecules contain oligodeoxynucleotide structures complementary to gene sequences in the target virus. Phosphorothioate oligonucleotides that are complementary to viral RNA have demonstrated inhibition of viral replication in cell cultures. ISIS 2922 is a phosphorothioate oligonucleotide with potent antiviral activity against CMV; it is complementary to the RNA of region 2 of the immediate early transcription unit of CMV and inhibits protein synthesis.

Interferons

Interferons are natural cellular products released from infected host cells in response to viral or other foreign nucleic acids. They are detectable as early as 2 h after infection. Their complex mechanism of action has not been fully established, but interferon selectively blocks translation and transcription of viral RNA stopping viral replication without disturbing normal host cell function.

Immunogens

The present inventions contemplates the use of the compositions and methods of the invention in combination with immunogenic compounds. The immunogenic or therapeutic agents, including proteins, polynucleotides and equivalents of the present invention may be administered as a sole active immunogen in an immunogenic composition or active in a therapeutic composition, or alternatively, the composition may include other active immunogens and/or therapeutics, including other immunogenic polynucleotides, polypeptides, or immunologically-active proteins of one or more other microbial pathogens (e.g. virus, prion, bacterium, or fungus, without limitation) or capsular polysaccharide. The compositions may comprise one or more desired proteins, fragments or pharmaceutical compounds as desired for a chosen indication. In the same manner, the compositions of this invention which employ one or more nucleic acids in the composition may also include nucleic acids which encode the same diverse group of proteins, as noted above.

The present invention contemplates the use of vector delivery and vector expression. For example, a vector or plasmid which expresses a protein or polypeptide of the present invention (e.g., B cell depleting agent, anti-cytokine agent, etc.) may be used to administer such protein or polypeptide to a patient. The protein or polypeptide of the present invention can be delivered in any suitable manner as known to persons skilled in the art. For example, the protein or polypeptide may be delivered using a live vector, in particular using live recombinant bacteria, viruses or other live agents, containing the genetic material necessary for the expression of the polypeptide or immunogenic portion as a foreign polypeptide.

Therapeutic Compositions and Administration

The present invention provides compositions and methods for treating patients with autoimmune conditions and those at risk of developing autoimmune conditions. The compositions of the present invention include therapeutic compositions for administration to subjects, preferably human subjects, as well as diagnostic and assay compositions. The compositions should preferably comprise a therapeutically effective amount of a conjugate of the invention. A suitable therapeutically effective amount for the purposes of the present invention as used is an amount of a therapeutic agent needed to treat, ameliorate or prevent a targeted disease or condition, or to exhibit a detectable therapeutic or preventative effect. For any B cell depleting agent, conjugate thereof and/or anti-cytokine agent, the therapeutically effective dose can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The methods of the present invention involve administering to the patient the agents and compositions of the present invention, such as B cell depleting agents, conjugates of B cell depleting agents and cytotoxic drugs and/or anti-cytokine agents. Preferably, the methods involve administering to the patient a cytotoxic drug/B cell depleting agent conjugate. More preferably, the conjugate is administered in combination with an anti-cytokine agent. Even more preferably, the conjugated B cell depleting agent is a humanized anti-CD22 antibody, the conjugated cytotoxic drug is calicheamicin and the anti-cytokine agent is an anti-TNF agent, such as etanercept.

The individual active agents can be administered either as part of the same composition, as separate compositions or in any combination. Preferably, when a B cell depleting agent or conjugate thereof is administered to the patient, the anti-cytokine is administered separately. The anti-cytokine agent may be administered at the same time or at different times as the B cell depleting agent or conjugate. The active agents may be administered alone, but are generally administered with a pharmaceutically acceptable diluent selected on the basis of the chosen route of administration and standard pharmaceutical practice.

Once formulated, the compositions of the invention can be administered directly to the subject. The subjects to be treated can be animals. However, it is preferred that the compounds and compositions are adapted for administration to human subjects.

The compositions of the present invention may be administered by any number of routes including, but not limited to, oral, intravenous, intramuscular, intra-arterial, intramedullary, intrathecal, intraventricular, transdermal, transcutaneous (see PCT Publication No. WO98/20734), subcutaneous, intraperitoneal, intranasal, enteral, topical, sublingual, intravaginal or rectal routes. Hyposprays may also be used to administer the compositions of the invention. Typically, the compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

The precise effective amount for administration to a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgment of the clinician. For example, an effective dose of a conjugate of the present invention will generally be from 0.1 mg/m² to 50 mg/m², preferably 0.4 mg/m² to 30 mg/m², more preferably 2 mg/m² to 9 mg/m², which dose is calculated on the basis of the B cell depleting agent of the conjugate.

Compositions may be administered individually to a patient or may be administered in combination with other agents, drugs or hormones. The dose at which the monomeric cytotoxic drug derivative/antibody conjugate of the present invention is administered depends on the nature of the condition to be treated, and on whether the conjugate is being used prophylactically or to treat an existing condition.

The frequency of dose will depend on the half-life of the conjugate and the duration of its effect. If the conjugate has a short half-life (e.g., 2 to 10 hours) it may be necessary to give one or more doses per day. Alternatively, if the conjugate molecule has a long half-life (e.g., 2 to 15 days) it may only be necessary to give a dosage once per day, once per week or even once every 1 or 2 months.

Preferably, the compositions contain a pharmaceutically acceptable diluent for administration of the antibody conjugate. The diluent should not itself induce the production of antibodies harmful to the individual receiving the composition and should not be toxic. Suitable diluents may be large, slowly metabolized macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulfates, or salts of organic acids, such as acetates, propionates, malonates and benzoates.

Pharmaceutically acceptable diluents in these compositions may additionally contain liquids such as water, saline, glycerol, and ethanol. Additionally, auxiliary substances, such as wetting or emulsifying agents or pH buffering substances, may be present in such compositions. Such diluents enable the compositions to be formulated as tablets, pills, dragees, capsules, liquids, gels, syrups, slurries or suspensions, for administration to the patient.

Preferred forms for administration include forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preserving, stabilizing and/or dispersing agents.

Although the stability of the buffered conjugate solutions is adequate for short-term stability, long-term stability is poor. To enhance stability of the conjugate and to increase its shelf life, the antibody-drug conjugate may be lyophilized to a dry form, for reconstitution before use with an appropriate sterile liquid. The problems associated with lyophilization of a protein solution are well documented. Loss of secondary, tertiary and quaternary structure can occur during freezing and drying processes. Consequently, cryoprotectants may have to be included to act as an amorphous stabilizer of the conjugate and to maintain the structural integrity of the protein during the lyophilization process. In one embodiment, the cryoprotectant useful in the present invention is a sugar alcohol, such as alditol, mannitol, sorbitol, inositol, polyethylene glycol, and combinations thereof. In another embodiment, the cryoprotectant is a sugar acid, including an aldonic acid, an uronic acid, an aldaric acid, and combinations thereof.

The cryoprotectant of this invention may also be a carbohydrate. Suitable carbohydrates are aldehyde or ketone compounds containing two or more hydroxyl groups. The carbohydrates may be cyclic or linear and include, for example, aldoses, ketoses, amino sugars, alditols, inositols, aldonic acids, uronic acids, or aldaric acids, or combinations thereof. The carbohydrate may also be a mono-, a di-, or a poly-carbohydrate, such as for example, a disaccharide or polysaccharide. Suitable carbohydrates include for example, glyceraldehydes, arabinose, lyxose, pentose, ribose, xylose, galactose, glucose, hexose, idose, mannose, talose, heptose, glucose, fructose, gluconic acid, sorbitol, lactose, mannitol, methyl α-glucopyranoside, maltose, isoascorbic acid, ascorbic acid, lactone, sorbose, glucaric acid, erythrose, threose, arabinose, allose, altrose, gulose, idose, talose, erythrulose, ribulose, xylulose, psicose, tagatose, glucuronic acid, gluconic acid, glucaric acid, galacturonic acid, mannuronic acid, glucosamine, galactosamine, sucrose, trehalose or neuraminic acid, or derivatives thereof. Suitable polycarbohydrates include, for example, arabinans, fructans, fucans, galactans, galacturonans, glucans, mannans, xylans (such as, for example, inulin), levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, or starch. Among particularly useful carbohydrates are sucrose, glucose, lactose, trehalose, and combinations thereof. Sucrose is a particularly useful cryoprotectant.

Preferably, the cryoprotectant of the present invention is a carbohydrate or “sugar” alcohol, which may be a polyhydric alcohol. Polyhydric compounds are compounds that contain more than one hydroxyl group. Preferably, the polyhydric compounds are linear. Suitable polyhydric compounds include, for example, glycols such as ethylene glycol, polyethylene glycol, and polypropylene glycol, glycerol, or pentaerythritol; or combinations thereof.

In some preferred embodiments, the cryoprotectant agent is sucrose, trehalose, mannitol, or sorbitol.

It will be appreciated that an active ingredient in certain embodiments of invention is a cytotoxic drug/B cell depleting agent conjugate. As such, it will be susceptible to degradation in the gastrointestinal tract. Thus, if the composition is to be administered by a route using the gastrointestinal tract, the composition will need to contain agents which protect the conjugate from degradation, but which release the conjugate once it has been absorbed from the gastrointestinal tract.

A thorough discussion of pharmaceutically acceptable carriers, diluents, etc is available in Remington's Pharmaceutical Sciences (Mack Publishing Company, N.J. 1991). The use of any such suitable carriers, diluents, etc. as would be known to persons skilled in the art is contemplated by the present invention.

The present invention in particular provides a monomeric calicheamicin derivative/humanized anti-CD22 antibody (G5/44) for use in treating proliferative disorders characterized by cells expressing CD22 antigen on their surface.

The present invention further provides the use of the monomeric calicheamicin derivative/humanized anti-CD22 antibody (G5/44) in the manufacture of a composition or a medicament for the treatment of a proliferative disorder characterized by cells expressing CD22.

The monomeric calicheamicin derivative/humanized anti-CD22 antibody (G5/44) may also be utilized in any therapy where it is desired to target cells expressing CD22 that are present in the subject being treated with the composition or a medicament disclosed herein. Specifically, the composition or medicament is used to treat humans or animals with an autoimmune disease. The CD22-expressing cells may be circulating in the body or be present in an undesirably large number localized at a particular site in the body.

Bioactive agents contemplated for use in the present invention include growth factors, cytokines, and cytotoxic drugs. Cytotoxic drugs which may be used together with the monomeric calicheamicin derivative/humanized anti-CD22 antibody (G5/44) include an anthracycline such as doxorubicin, daunorubicin, idarubicin, aclarubicin, zorubicin, mitoxantrone, epirubicin, carubicin, nogalamycin, menogaril, pitarubicin, and valrubicin for up to three days; and a pyrimidine or purine nucleoside such as cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin. Other chemotherapeutic/antineoplastic agents that may be administered in combination with the conjugate include adriamycin, cisplatin, carboplatin, cyclophosphamide, dacarbazine, vinblastine, vincristine, mitoxantrone, bleomycin, mechlorethamine, prednisone, procarbazine, methotrexate, fluorouracils, etoposide, taxol and its various analogs, and mitomycin. The monomeric calicheamicin derivative/humanized anti-CD22 antibody (G5/44) may be administered concurrently with one or more of these therapeutic agents. Alternatively, the conjugate may be administered sequentially with one or more of these therapeutic agents.

The B cell depleting agent, cytotoxic drug, cytotoxic drug/B cell depleting agent conjugate and/or anti-cytokine agent may be administered alone, concurrently, or sequentially with a combination of other bioactive agents such as growth factors, cytokines, steroids, antibodies such as anti-CD20 antibody, rituximab (Rituxan™), and chemotherapeutic agents as a part of a treatment regimen. Treatment regimens are contemplated by the present invention, such as CHOPP (cyclophosphamide, doxorubicin, vincristine, prednisone, and procarbazine), CHOP (cyclophosphamide, doxorubicin, vincristine, and prednisone), COP (cyclophosphamide, vincristine, and prednisone), CAP-BOP (cyclophosphamide, doxorubicin, procarbazine, bleomycin, vincristine, and prednisone), m-BACOD (methotrexate, bleomycin, doxorubicin, cyclophosphamide, vincristine, dexamethasone, and leucovorin), ProMACE-MOPP (prednisone, methotrexate, doxorubicin, cyclophosphamide, etoposide, leucovorin, mechloethamine, vincristine, prednisone, and procarbazine), ProMACE-CytaBOM (prednisone, methotrexate, doxorubicin, cyclophosphamide, etoposide, leucovorin, cytarabine, bleomycin, and vincristine), MACOP-B (methotrexate, doxorubicin, cyclophosphamide, vincristine, fixed dose prednisone, bleomycin, and leucovorin), MOPP (mechloethamine, vincristine, prednisone, and procarbazine), ABVD (adriamycin/doxorubicin, bleomycin, vinblastine, and dacarbazine), MOPP alternating with ABV (adriamycin/doxorubicin, bleomycin, and vinblastine), and MOPP (mechloethamine, vincristine, prednisone, and procarbazine) alternating with ABVD (adriamycin/doxorubicin, bleomycin, vinblastine, and dacarbazine), and ChIVPP (chlorambucil, vinblastine, procarbazine, and prednisone). Therapy may comprise an induction therapy phase, a consolidation therapy phase and a maintenance therapy phase. The B cell depleting agent, cytotoxic drug, cytotoxic drug/B cell depleting agent conjugate and/or anti-cytokine agent may also be administered alone, concurrently, or sequentially with any of the above identified therapy regimens as a part of induction therapy phase, a consolidation therapy phase and a maintenance therapy phase.

The conjugates of the present invention may also be administered together with other bioactive and chemotherapeutic agents as a part of combination regimen. Such a treatment regimen includes IMVP-16 (ifosfamide, methotrexate, and etoposide), MIME (methyl-gag, ifosfamide, methotrexate, and etoposide), DHAP (dexamethasone, high-dose cytaribine, and cisplatin), ESHAP (etoposide, methylpredisolone, high-dose cytarabine, and cisplatin), EPOCH (etoposide, vincristine, and doxorubicin for 96 hours with bolus doses of cyclophosphamide and oral prednisone), CEPP(B) (cyclophosphamide, etoposide, procarbazine, prednisone, and bleomycin), CAMP (lomustine, mitoxantrone, cytarabine, and prednisone), CVP-1 (cyclophosphamide, vincristine and prednisone), CHOP-B. (cyclophosphamide, doxorubicin, vincristine, prednisone, and Bleomycin), CEPP-B (cyclophosphamide, etoposide, procarbazine, and bleomycin), and P/DOCE (epirubicin or doxorubicin, vincristine, cyclophosphamide, and prednisone) Additional treatment regimens for may include in phase 1 a first line of treatment with CHOP (cyclophosphamide, doxorubicin, vincristine, and prednisone)-rituximab (Rituxan™)-CMC-544 (CMC-544 is described in U.S. Patent Application No. US 2004/082764 and PCT publication WO 03/092623 which are incorporated by reference in their entirety), followed in phase 2 and phase 3 with CHOP-rituximab (Rituxan™), CHOP-CMC-544 or CHOP-rituximab (Rituxan™)-CMC-544. Alternatively, phase 1 may have a first line of treatment with COP (cyclophosphamide, vincristine, and prednisone)-rituximab (Rituxan™)-CMC-544, followed in phase 2 and phase 3 with COP-rituximab (Rituxan™), COP-CMC-544 or COP-rituximab (Rituxan™)-CMC-544. In a further embodiment, treatment may include a first or second line of treatment with the antibody drug conjugate CMC-544 in phase 1, followed in phase 2 and 3 with CMC-544 and CHOP (cyclophosphamide, doxorubicin, vincristine, and prednisone), CMC-544 and COP (cyclophosphamide, vincristine, and prednisone), CMC-544 with rituximab (Rituxan™) or rituximab (Rituxan™) alone. In yet another embodiment, the treatment may include a first or line of treatment with the antibody drug conjugate CMC-544 followed in phase 2 and 3 with CMC-544 alone or in combination with other treatment regimens including, but not limited to, ESHOP (etoposide, methylpredisolone, high-dose cytarabine, vincristine and cisplatin), EPOCH (etoposide, vincristine, and doxorubicin for 96 hours with bolus doses of cyclophosphamide and oral prednisone), IMVP-16 (ifosfamide, methotrexate, and etoposide), ASHAP (Adriamycin, solumedrol, Ara-C, and cisplatin), MIME (methyl-gag, ifosfamide, methotrexate, and etoposide) and ICE (ifosfamide, cyclophosphamide, and etoposide). Details of various cytotoxic drugs can be found in Cancer Principles and Practice of Oncology, Eds. Vincent T. DeVita, Samuelo Hellman, Steven A. Rosenberg, 6^{th} Edition, Publishers: Lippincott, Williams and Wilkins (2001) and Physician's Cancer Chemotherapy Drug Manual, Eds. Edward Chu and Vincent T. DeVita, Publishers: Jones and Bartlett, (2002).

The formulation of such compositions is well known to persons skilled in this field. Compositions of the invention preferably include pharmaceutically acceptable diluents (i.e., drug delivery systems). Suitable pharmaceutically acceptable diluents include any and all conventional solvents, dispersion media, fillers, solid carriers, aqueous solutions, coatings, antibacterial and antifungal agents, isotonic and absorption delaying agents, and the like. Suitable pharmaceutically acceptable diluents include, for example, one or more of water, saline, phosphate buffered saline, dextrose, glycerol, ethanol and the like, as well as combinations thereof. Pharmaceutically acceptable diluents may further comprise minor amounts of auxiliary substances such as wetting or emulsifying agents, preservatives or buffers, which enhance the shelf life or effectiveness of the antibody. The preparation and use of pharmaceutically acceptable diluents is well known in the art. Except insofar as any conventional media or agent is incompatible with the active ingredient, use thereof in the compositions of the present invention is contemplated.

Such therapeutic compositions can be administered parenterally, e.g., by injection, either subcutaneously or intramuscularly, as well as orally or intranasally. Methods for intramuscular immunization are described by Wolff et al. and by Sedegah et al. Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium carbonate, and the like, without limitation.

The present invention also provides a process for the preparation of a therapeutic or diagnostic composition/formulation comprising admixing the monomeric cytotoxic drug/B cell depleting agent conjugate of the present invention together with a pharmaceutically acceptable excipient, diluent or carrier.

The monomeric cytotoxic drug/B cell depleting agent conjugate may be the sole active ingredient in the therapeutic or diagnostic composition/formulation or may be accompanied by other active ingredients including other antibody ingredients, for example anti-CD19, anti-CD20, anti-T cell, anti-IFNγ or anti-LPS antibodies, or non-antibody ingredients such as anti-cytokine agents, such as anti-TNF agents (e.g., etanercept), growth factors, hormones, anti-hormones, cytotoxic drugs and xanthines.

Cytokines and growth factors which may be used together with the cytotoxic drug derivative/B cell depleting agent conjugates of the present invention include interferons, interleukins such as interleukin 2 (IL-2), TNF, CSF, GM-CSF and G-CSF.

Hormones which may be used together with the cytotoxic drug derivative/B cell depleting agent conjugates of the present invention include estrogens such as diethylstilbestrol and estradiol, androgens such as testosterone and Halotestin, progestins such as Megace and Provera, and corticosteroids such as prednisone, dexamethasone, and hydrocortisone.

Antihormones such as antiestrogens, i.e., tamoxifen, antiandrogens, i.e., flutamide and antiadrenal agents may be used together with the cytotoxic drug derivative/B cell depleting agent conjugate of the present invention.

The compositions of the invention can include an immunogen. Immunogen compostions can include one or more adjuvants, including, but not limited to aluminum hydroxide; aluminum phosphate; STIMULON™ QS-21 (Aquila Biopharmaceuticals, Inc., Framingham, Mass.); MPL™ (3-O-deacylated monophosphoryl lipid A; Corixa, Hamilton, Mont.), 529 (an amino alkyl glucosamine phosphate compound, Corixa, Hamilton, Mont.), IL-12 (Genetics Institute, Cambridge, Mass.); GM-CSF (Immunex Corp., Seattle, Wash.); N-acetyl-muramyl-L-theronyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphos-phoryloxy-ethylamine) (CGP 19835A, referred to as MTP-PE); and cholera toxin. Others which may be used are non-toxic derivatives of cholera toxin, including its A subunit, and/or conjugates or genetically engineered fusions of the polypeptide with cholera toxin or its B subunit (“CTB”), procholeragenoid, fungal polysaccharides, including schizophyllan, muramyl dipeptide, muramyl dipeptide (“MDP”) derivatives, phorbol esters, the heat labile toxin of E. coli, block polymers or saponins.

As with the conjugates of the present invention, the dosage of the anti-cytokine agent administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

For example, usually the daily dosage of an anti-cytokine agent, such as the anti-TNF agent etanercept, is about 0.01 to 100 milligrams per kilogram of body weight. Ordinarily 1.0 to 5, and preferably 1 to 10 milligrams per kilogram per day given in divided doses 1 to 6 times a day or in sustained release form is effective to obtain desired results.

As a non-limiting example, treatment can be provided as a daily dosage of anti-cytokine agent, such as anti-TNF peptides, monoclonal chimeric and/or routine antibodies of the present invention of about 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every 24, 12, 8, 6, 4 or 2 hours, or any combination thereof.

Since circulating concentrations of TNF tend to be extremely low, in the range of about 10 pg/ml in non-septic individuals, and reaching about 50 pg/ml in septic patients and above 100 pg/ml in the sepsis syndrome (Hammerle, A. F. et al., 1989, infra) or can only be detectable at sites of TNF-mediated pathology, it is preferred to use high affinity and/or potent in vivo TNF-inhibiting and/or neutralizing antibodies, fragments or regions thereof, for both TNF immunoassays and therapy of TNF-mediated pathology. Such antibodies, Fragments, or regions, will preferably have an affinity for hTNF-α, expressed as Ka, of at least 10⁸ M.sup.⁻¹, more preferably, at least 10⁹ M⁻¹, such as 10⁸-10¹⁰ M⁻¹, 5×10⁸ M⁻¹, 8×10⁸ M⁻¹, 2×10⁹ M⁻¹, 4×10⁹ M⁻¹, 6×10⁹ M⁻¹, 8×10⁹ M⁻¹, or any range or value therein.

Preferred for human therapeutic use are high affinity murine and chimeric antibodies, and fragments, regions and derivatives having potent in vivo TNF-α-inhibiting and/or neutralizing activity, according to the present invention, that block TNF-induced IL-6 secretion. Also preferred for human therapeutic uses are such high affinity murine and chimeric anti-TNF-α antibodies, and fragments, regions and derivatives thereof, that block TNF-induced procoagulant activity, including blocking of TNF-induced expression of cell adhesion molecules such as ELAM-1 and ICAM-1 and blocking of TNF mitogenic activity, in vivo, in situ, and in vitro.

The compositions of the present invention preferably include a pharmaceutically acceptable carrier. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the composition. The preparation and use of pharmaceutically acceptable carriers is well known in the art. Except insofar as any conventional media or agent is incompatible with the active ingredient, use thereof in the compositions of the present invention is contemplated.

The present compositions can be administered parenterally, e.g., by injection, either subcutaneously or intramuscularly, for example, as well as orally or intranasally. Methods for intramuscular injection are described by Wolff et al., and by Sedegah et al. Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium, carbonate, and the like, without limitation

Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

For parenteral administration, anti-cytokine agents, for example, anti-TNF peptides or antibodies, can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques.

Suitable pharmaceutical carriers are described in the most recent edition of Remington's Pharmaceutical Sciences, A. Osol, a standard reference text in this field of art. The compositions and methods of the present invention may be used in combination with other therapies, such as supportive therapy, for example, in accordance with an implementation of the present invention.

According to an implementation of the present invention, a composition of the invention may be administered to a patient along with intravenous (IV) fluids. For example, the present compositions may be contained within the intravenous (IV) bag or may be injected into the lock of intravenous (IV) line.

In another implementation, the composition of the present invention may be administered to a patient along with oxygen or other such treatment. For example, a composition of the invention may be administered via a nebulizer.

For example, a parenteral composition suitable for administration by injection is prepared by dissolving 1.5% by weight of active ingredient in 0.9% sodium chloride solution.

Any efficacious route of administration may be used to therapeutically administer the active agents. If injected, the inhibitors can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, and topical preparations such as lotions, gels, sprays, ointments or other suitable techniques. Alternatively, proteinaceous anti-cytokine agents, such as a soluble TNFR, may be administered by implanting cultured cells that express the protein. When the inhibitor is administered in combination with one or more other biologically active compounds, these may be administered by the same or by different routes, and may be administered simultaneously, separately or sequentially.

Anti-cytokine agents, such as TNFR:Fc or other soluble TNFRs, preferably are administered in the form of a physiologically acceptable composition comprising purified recombinant protein in conjunction with physiologically acceptable carriers, excipients or diluents. Such carriers are nontoxic to recipients at the dosages and concentrations employed. Ordinarily, the preparation of such compositions entails combining the anti-cytokine agent, such as anti-TNF-α agents with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents. In accordance with appropriate industry standards, preservatives may also be added, such as benzyl alcohol. TNFR:Fc preferably is formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16.sup.th Ed., Mack Publishing Company, Easton, Pa., 1980.

Appropriate dosages can be determined in standard dosing trials, and may vary according to the chosen route of administration. The amount and frequency of administration will depend on such factors as the nature and severity of the indication being treated, the desired response, the age and condition of the patient, and so forth.

An anti-cytokine agent such as TNFR:Fc is preferably administered one time per week, more preferably, at least two times per week, and even more preferably at least three times per week. An adult patient is a person who is 18 years of age or older. If injected, the effective amount of TNFR:Fc per adult dose ranges from 1-20 mgm.sup.2, and preferably is about 5-12 mg/m.sup.2. Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing TNFR:Fc at 25 mg/dose, or alternatively, containing 50 mg per dose. The 25 mg or 50 mg dose may be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of TNFR:Fc one to three times per week over a period of at least three weeks, or a dose of 50 mg of TNFR:Fc one or two times per week for at least three weeks, though treatment for longer periods may be necessary to induce the desired degree of improvement. For incurable chronic conditions, the regimen may be continued indefinitely, with adjustments being made to dose and frequency if such are deemed necessary by the patient's physician.

For pediatric patients (age 4-17), a suitable regimen involves the subcutaneous injection of 0.4 mg/kg, up to a maximum dose of 25 mg of TNFR:Fc, administered by subcutaneous injection one or more times per week.

The invention further includes the administration of anti-cytokine agents concurrently with one or more other drugs that are administered to the same patient, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components may be administered in the same or in separate compositions, and by the same or different routes of administration. Examples of drugs to be administered concurrently include but are not limited to antivirals, antibiotics, analgesics, corticosteroids, DMARDs and non-steroidal anti-inflammatories. DMARDs that can be administered include azathioprine, cyclophosphamide, cyclosporine, hydroxychloroquine sulfate, methotrexate, leflunomide, minocycline, penicillamine, sulfasalazine and gold compounds such as oral gold, gold sodium thiomalate and aurothioglucose.

An anti-cytokine agent may be combined with one or more additional anti-cytokine agents. For example, TNFR:Fc may be combined with a second anti-TNF-α agent, including an antibody against TNF-α or TNFR, a TNF-α derived peptide that acts as a competitive inhibitor of TNF-α (such as those described in U.S. Pat. No. 5,795,859 or U.S. Pat. No. 6,107,273), a TNFR-IgG fusion protein other than etanercept, such as one containing the extracellular portion of the p55 TNF-α receptor, a soluble TNFR other than an IgG fusion protein, or other molecules that reduce endogenous TNF-α levels such as inhibitors of the TNF-α converting enzyme (see e.g., U.S. Pat. No. 5,594,106), or any of the small molecules or TNF-α inhibitors that are described above, including pentoxifylline or thalidomide.

If an antibody against TNF-α is used as the anti-TNF-α agent, a preferred dose range is 0.1 to 20 mg/kg, and more preferably is 1-10 mg/kg. Another preferred dose range for the anti-TNF-α antibody is 0.75 to 7.5 mg/kg of body weight. Humanized antibodies (i.e., antibodies in which only the antigen-binding portion of the antibody molecule is derived from a non-human source) are preferred. An exemplary humanized antibody for treating the hereindescribed diseases is infliximab (sold by Centocor as REMICADE) which is a chimeric IgG1-κ monoclonal antibody having an approximate molecular weight of 149,100 daltons. Infliximab is composed of human constant and murine variable regions, and binds specifically to human TNF-α. Other suitable anti-TNF-α antibodies include the humanized antibodies D2E7 and CDP571, and the antibodies described in EP 0 516 785 B1. U.S. Pat. No. 5,656,272, EP 0 492 448 A1. Such antibodies may be injected or administered intravenously.

In one preferred embodiment of the invention, the various medical disorders disclosed herein as being treatable with anti-TNF-α agent are treated in combination with another anti-cytokine agent. For example, a soluble TNFR such as TNFR:Fc may be administered in a composition that also contains a compound that inhibits the interaction of other inflammatory cytokines with their receptors. Examples of cytokine inhibitors used in combination with TNFR:Fc include, for example, antagonists of TNF-beta, IL-6 or IL-8. TNF-α inhibitors such as TNFR:Fc also may be administered in combination with the cytokines GM-CSF, IL2 and inhibitors of protein kinase A type 1 to enhance T cell proliferation in HIV-infected patients who are receiving anti-retroviral therapy. In addition, TNF-α inhibitors may be combined with inhibitors of IL-13 to treat Hodgkin's disease.

Other combinations for treating the hereindescribed diseases include TNFR:Fc administered concurrently with compounds that are antivirals.

In addition, the subject invention provides methods for treating a human patient in need thereof, the method involving administering to the patient a therapeutically effective amount of an anti-TNF agent and an IL-6 inhibitor.

The present invention also relates to the use of the disclosed anti-cytokines such as TNFR:Fc in the manufacture of a medicament for the prevention or therapeutic treatment of autoimmune diseases.

The present invention thus provides anti-TNF compounds and compositions comprising anti-TNF antibodies (Abs) and/or anti-TNF peptides which inhibit and/or neutralize TNF biological activity in vitro, in situ and/or in vivo, as specific for association with neutralizing epitopes of human tumor necrosis factor-alpha (hTNF-α) and/or human tumor necrosis factor .beta. (hTNF-beta). Such anti-TNF Abs or peptides have utilities for use in treating autoimmune diseases.

Anti-TNF peptides and/or antibodies of this invention can be adapted for therapeutic efficacy by virtue of their ability to mediate antibody-dependent cellular cytotoxicity (ADCC) and/or complement-dependent cytotoxicity (CDC) against cells having TNF associated with their surface. For these activities, either an endogenous source or an exogenous source of effector cells (for ADCC) or complement components (for CDC) can be utilized. The murine and chimeric antibodies, fragments and regions of this invention, their fragments, and derivatives can be used therapeutically as immunoconjugates (see for review: Dillman, R. O., Ann. Int. Med. 111:592-603 (1989)). Such peptides or Abs can be coupled to cytotoxic proteins, including, but not limited to ricin-A, Pseudomonas toxin and Diphtheria toxin. Toxins conjugated to antibodies or other ligands or peptides are well known in the art (see, for example, Olsnes, S. et al., Immunol. Today 10:291-295 (1989)). Plant and bacterial toxins typically kill cells by disrupting the protein synthetic machinery.

Anti-cytokines, such as anti-TNF peptides and/or antibodies, of this invention can be conjugated to additional types of therapeutic moieties including, but not limited to, radionuclides, therapeutic agents, cytotoxic agents and drugs. Examples of radionuclides which can be coupled to antibodies and delivered in vivo to sites of antigen include .sup.212 Bi.sup.132 I, .sup. 186 Re, and .sup.90 Y, which list is not intended to be exhaustive. The radionuclides exert their cytotoxic effect by locally irradiating the cells, leading to various intracellular lesions; as is known in the art of radiotherapy.

Cytotoxic drugs which can be conjugated to anti-cytokines, such as anti-TNF peptides and/or antibodies, and subsequently used for in vivo therapy include, but are not limited to, daunorubicin, doxorubicin, methotrexate, and Mitomycin C. Cytotoxie drugs interfere with critical cellular processes including DNA, RNA, and protein synthesis. For a description of these classes of drugs which are well known in the art, and their mechanisms of action, see Goodman, et al., Goodman and Gilman's THE PHARMACOLOGICAL BASIS OF THERAPEUTICS, 8th Ed., Macmillan Publishing Co., 1990.

Anti-cytokines, such as anti-TNF peptides and/or antibodies, of this invention can be advantageously utilized in combination with other monoclonal or routine mad chimeric antibodies, fragments and regions, or with lymphokines or hemopoietic growth factors etc., which serve to increase the number or activity of effector cells which interact with the antibodies.

Anti-TNF peptides and/or antibodies, fragments or derivatives of this invention can also be used in combination with TNF therapy to block undesired side effects of TNF. For example, recent approaches to cancer therapy have included direct administration of TNF to cancer patients or immunotherapy of cancer patients with lymphokine activated killer (LAK) cells (Rosenberg et al., New Eng. J. Med. 313:1485-1492 (1985)) or tumor infiltrating lymphocytes (TIL) (Kurnick et al. (Clin. Immunol Immunopath. 38:367-380 (1986); Kradin et al., Cancer Immunol. Immunother. 24:76-85 (1987); Kradin et al., Transplant. Proc. 20:336-338 (1988)). Trials are currently underway using modified LAK cells or TIL which have been transfected with the TNF gene to produce large amounts of TNF. Such therapeutic approaches are likely to be associated with a number of undesired side effects caused by the pleiotropic actions of TNF as described herein and known in the related arts. According to the present invention, these side effects can be reduced by concurrent treatment of a subject receiving TNF or cells producing large amounts of TIL with the antibodies, fragments or derivatives of the present invention. Effective doses are as described above. The dose level will require adjustment according to the dose of TNF or TNF-producing cells administered, in order to block side effects without blocking the main anti-tumor effect of TNF. A person of ordinary skill in the art would know how to determine such doses without undue experimentation.

The present invention contemplates the treatment of any autoimmune disease and the like. Non-limiting exemplary autoimmune diseases include alopecia greata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, Celiac Sprue-dermatitis, chronic fatigue syndrome immune deficiency syndrome (CFIDS), chronic inflammatory demyelinating polyneuropathy, cicatricial pemphigoid, cold agglutinin disease, CREST syndrome, Crohn's disease, Dego's disease, dermatomyositis, dermatomyositis—juvenile, discoid lupus, essential mixed cryoglobulinemia, fibromyalgia—fibromyositis, Grave's disease, Guillain-Barre, Hashimoto's thyroiditis, idiopathic pulmonary fibrosis, idiopathic thrombocytopenia purpura (ITP), IgA nephropathy, immune cytopenias, insulin dependent diabetes (Type I), juvenile arthritis, lupus, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, Stiff-Man syndrome, systemic lupus, Takayasu arteritis, temporal arteritis/giant cell arteritis, ulcerative colitis, uveitis, vasculitis, vitiligo, and Wegener's granulomatosis.

Preferably, the methods of the present invention are directed to treatment of rheumatoid arthritis (RA), systemic lupus (SLE), immune cytopenias (e.g., idiopathic thrombocytopenic purpura and autoimmune hemolytic anemia), and/or autoimmune vasculitis in humans.

Screening Methods

The present invention contemplates screening methods for identifying agents, compositions and treatments effective against autoimmune diseases. In accordance with an implementation, a screening method comprises: administering a candidate treatment to an animal model and monitoring the effectiveness of the treatment. Preferably, the animal model is a CIA mouse model. According to another implementation, a screening method comprises administering a candidate treatment to a group of patients with an autoimmune disease in a randomized placebo study; and monitoring the effectiveness of the treatment.

The description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art. A person skilled in the art would know, or be able to ascertain, using no more than routine experimentation, many equivalents to the specific embodiments of the invention described herein, based upon the guidance provided herein.

The following examples are included to demonstrate preferred embodiments of the invention. It should be appreciated by those skilled in the art that the techniques disclosed in the examples which follow represent techniques discovered by the inventors to function well in the practice of the invention, and thus can be considered to constitute preferred modes for its practice. However, those of skill in the art should, in view of the present disclosure, appreciate that many changes can be made in the specific embodiments which are disclosed and still obtain a like or similar result without departing from the spirit and scope of the invention. The following examples are offered by way of illustration and are not intended to limit the invention in any way.

### EXAMPLES

### Example 1

### B Cell Depletion with Anti-CD22/Calicheamicin Immunoconjugate Inhibits Collagen-induced Arthritis in a C57BL/6 Mouse Model

A study was conducted to test the role of B cell depletion in a mouse model of rheumatoid arthritis. The B cell depleting compound used in the study was a mouse anti-CD22 mAb (Cy34.12) conjugated to calicheamicin (“the conjugate”), a member of the enediyne antitumor antibiotics.

Because of the Cy34.12 reactivity, mice on C57BL/6 background were used. For in vitro cytotoxicity experiments, purified primary B cells from male C57BL/6 mice were cultured with the conjugate and their proliferation in response to LPS stimulation was studied 48 hours after culture initiation.

For in vivo cytotoxicity studies, male C57BL/6 mice received two (day 0 and 5) intraperitoneal (i.p.) injections with the conjugate, at a calicheamicin dose of 160 pg/kg/injection. B cell depletion was monitored with flow cytometry in bone marrow (BM), spleen, lymph node (LN), and peripheral blood (PB) serial samples. Collagen-induced arthritis (CIA) was induced in male C57BL/6 IFN-g KO mice by one (day 0) intradermal immunization with bovine type II collagen (CII) in complete Freund's adjuvant (CFA). CII immunized mice received two i.p. injections (day 5 and 10) with the conjugate, at a calicheamicin dose of 160 pg/kg/injection. The paws were evaluated for clinical signs of arthritis using a semiquantitative scoring system (0-4). Mice were sacrificed at various time points after immunization and paws were collected for histologic analysis.

The study showed that the conjugate has selective in vitro cytotoxicity for CD22+ B cells at very low concentrations (average IC 50: 0.08 p,g/ml of conjugated antibody). Two i.p. injections of naive mice with the conjugate result in selective cytotoxicity of CD22+ B cells, but not of CD3+ T cells and GR-I+ myeloid cells, in all tissues tested on days 12, 20, and 30. Numbers of B cells start to increase in depleted mice around day 35, and there is complete B cell recovery at day 50 post injections. In the CIA model, treatment of C57BL/6 IFN-7 KO with the conjugate on days 5 and 10 after immunization with CII protected them from the development of clinical and histologic arthritis. B cell depleted mice remained without clinical arthritis even after complete recovery of the B cell pool.

From the study, it can be concluded that treatment of CII immunized mice with anti-CD22/calicheamicin effectively inhibits arthritis-related clinical and histologic manifestations. The protective effect is connected with in vivo depletion of B cells and validates the pathogenic role of B cells in collagen-induced arthritis.

### Example 2

### CD22-Targeted B Cell Depletion Inhibits Clinical and Histological Arthritis in a Collagen-Induced Arthritis (CIA) Model

A study was conducted to test the role of B cell depletion in a collagen-induced arthritis (CIA) model. The B cell depleting compound (referred to herein as CD22/cal) used in the study was a conjugate of an anti-mouse CD22 monoclonal antibody (mAb) and N-acetyl gamma calicheamicin dimethyl acid, a member of the enediyne antitumor antibiotics. Anti-mouse CD22 is a mouse IgG1 mAb purified from Cy34.1 hybridoma (American Type Culture Collection, Rockville, Md.). The synthesis of antibody/calicheamicin conjugates has been previously described. Hamann, P. R. et al. An anti-CD33 antibody-calicheamicin conjugate for treatment of acute myeloid leukemia. Choice of linker. Bioconjug Chem 13, 40-6 (2002). CD22/cal has an average loading of 17 to 30 μg calicheamicin/mg antibody protein (1.2-2.6 moles calicheamicin/mol antibody). Upon binding to CD22 expressing mouse B cells, the conjugate is internalized and exhibits potent dose-dependent cytotoxicity due to DNA damage caused by calicheamicin. Thorson, J. S. et al. Understanding and exploiting nature's chemical arsenal: the past, present and future of calicheamicin research. Curr Pharm Des 6, 1841-79 (2000). Damle, N. K. & Frost, P. Antibody-targeted chemotherapy with immunoconjugates of calicheamicin. Curr Opin Pharmacol 3, 386-90 (2003). A mouse IgG1 mAb conjugated to calicheamicin (J110/cal) was used as a control in in-vitro cytotoxicity assays. Mouse A20 B cell lymphoma cells (American Type Culture Collection) were used for flow cytometry studies on binding of Cy34.1 and CD22/cal on mouse CD22.

Female and male C57BL/6 (B6) and female IFNγ^{-/-} in B6 background (B6-IFNγ-KO), 6 to 8 weeks old, were purchased from Jackson Laboratories (Bar Harbor, Me.). The animals were kept at the animal facility of Wyeth Research in accordance with the guidelines of the Committee on the Care and Use of Laboratory Animals of the Institute of Laboratory Resources, National Research Council.

In-vitro B and T cell cytotoxicity studies were conducted. Primary mouse B cells were purified from single cell splenocyte suspension using CD19 Microbeads (Miltenyi Biotec, Auburn, Calif.) according to the manufacturer's instructions. For in-vitro cytotoxicity (IC₅₀) studies, purified primary B cells (10⁵ cells/well) from male B6 mice were cultured in a 96-well plate with various concentrations of the conjugate and their proliferation in response to 50 μg/ml LPS (E coli 026:B6, L 2762; SIGMA,) stimulation was studied 48 hours after culture initiation. ³H thymidine at 1 μCi/well (PerkinElmer Life Sciences, Boston, Mass.) was added during the last 6 hours of culture. After harvesting the supernatant onto glass fiber filter mats, ³H-thymindine incorporation was determined by liquid scintillation counting. Mouse primary T cells were purified from single cell splenocyte suspension using CD3 Microbeads (Miltenyi Biotec). Purified T cells (10⁵ cells/well) were cultured in a 96-well plate with various concentrations of the conjugate and their proliferation in response to suboptimal (500 ng/ml) concentration of soluble anti-CD3 mAb (145-2C11, PharMingen, San Diego, Calif.) plus 1 μg/ml anti-CD28 mAb (clone 37.51, PharMingen) was studied 48 hours after culture initiation. ³H thymidine at 1 μCi/well was added during the last 6 hours of culture.

In-vivo B cell cytotoxicity studies were conducted. Male B6 mice were used for the establishment of the in-vivo protocol and the characterization of B cell depletion and recovery. Several CD22/cal dosing protocols were tested, and the most effective one was used for further studies. According to this protocol, male B6 mice received two (day 0 and 5) intraperitoneal (i.p.) injections with the conjugate at a calicheamicin dose of 160 μg/kg/injection. B cell depletion was monitored in individual mice by flow cytometry in bone marrow (BM), spleen, lymph node (LN), and peripheral blood (PB) samples on days 12, 20, 30, 35, and 50 after the first injection. Three mice/per time point were studied and representative flow cytometry data from individual mice are shown.

Flow cytometry was used to analyze the cells. The following fluorescein isothiocyanate (FITC) or phycoerythrin (PE) conjugated antibodies directed to mouse cell-surface antigens were from BD Pharmingen (San Jose, Calif.): CD3e (145-2C11), CD19 (1D3), CD22.2 (Cy34.1), CD45R/B220 (RA3-6B2), Gr-1 (RB6-8C5), and Mac-3 (M3/84). For staining of A20 cells with unconjugated Cy34.1 or Cy34.1/calicheamicin (CD22/cal), anti-mouse IgG1-biotin and streptavidin PE polyclonal antibody was used. Cells were analyzed by flow cytometry using FACSCalibur and CellQuest software package (BD PharMingen).

The DBA/1 strain (Lyb-8.1) murine model of CIA could not be used in our studies because Cy34.12 mAb reacts with CD22 on strains expressing the Lyb-8.2 alloantigen. Therefore, we used a CIA model on the B6 background. CIA was induced according to the protocol by Chu et al. Chu, C. Q., Song, Z., Mayton, L., Wu, B. & Wooley, P. H. IFNgamma deficient C57BL/6 (H-2b) mice develop collagen induced arthritis with predominant usage of T cell receptor Vbeta6 and Vbeta8 in arthritic joints. Ann Rheum Dis 62, 983-90 (2003). Briefly, CIA was induced in male B6 IFN-γ KO mice by one (day 0) intradermal immunization with 100 μg bovine type II collagen (CII) in complete Freund's adjuvant (CFA) (Difco Laboratory, Detroit, Mich.), containing 5 mg/ml of killed Mycobacterium tuberculosis (H37Ra). CII immunized mice received two i.p. injections (day 5 and 10) with the conjugate, at a calicheamicin dose of 160 μg/kg/injection. Control mice were immunized with CII in CFA, as described, and injected i.p. with 200 μl of phosphate buffered saline (PBS) on days 5 and 10. The paws were evaluated for clinical signs of arthritis using a semi-quantitative scoring system (0-4). Mice were sacrificed at various time points after immunization and paws were collected for histological analysis.

The paws were fixed in 10% neutral buffered formalin and decalcified in Cal-Ex II (Fisher Scientific) for 10 days. Paws were routinely processed and then embedded in paraffin blocks. Specimens were sectioned at 6 μm and stained with hematoxylin and eosin according to the manufacturer's protocol (Sigma-Aldrich). The sections were microscopically evaluated for the degree of inflammatory cell infiltration, cartilage degeneration and erosion, synovial hyperplasia and pannus formation, and bone degeneration and remodeling. The arthritis severity of the disease in each paw was graded using a scoring system from 0 to 4: 0=within normal limits; 1=slight/mild; 2=moderate; 3=marked; 4=severe. The score assigned to each paw reflected the overall extend and severity of involvement of the many joints represented on each slide.

B cell depletion in the RSV vaccination model was observed for comparison. Four groups of female B6 (age 7-9 weeks) mice were administered vaccine and/or conjugate according to the protocol depicted in Table 2. Mice from groups 1 and 2 were immunized intramuscularly (i.m.) with the RSV fusion (F) protein (1 μg/dose) adsorbed to aluminum phosphate (AIPO) adjuvant (100 μg/dose) on weeks 0 and 2. Natural F protein was purified as previously described, Hancock, G. E. et al. Generation of atypical pulmonary inflammatory responses in BALB/c mice after immunization with the native attachment (G) glycoprotein of respiratory syncytial virus. J Virol, 70, 7783-91 (1996), from Vero cells (ATCC No. CCL 81) infected with the A2 strain of RSV. The protein was greater than 95% pure as estimated by SDS-PAGE and antigen capture ELISA. Mice in groups 3 and 4 were not vaccinated. On weeks 4 and 4 plus 5 days mice in groups 1 and 3 were injected i.p. with the CD22/cal conjugate (160 μg/kg). Control mice were injected with PBS. Flow cytometric analysis was performed on peripheral blood samples collected prior to and 9 days after secondary treatment with the conjugate. On week 12 plus 4 days all mice were challenged intranasally (i.n.) with ˜10⁶ PFU RSV (A2 strain). Sera were collected on week 0, 2, 4, 8, 12, 14, 25 and ELISAs were performed to ascertain serum anti-F protein IgG and IgM titers. To accommodate frequency of bleeding, groups were composed of 10 mice such that, each data point represents geometric mean titers of 5 mice/group.

RSV infectivity was determined. The detection of infectious virus in the lungs after challenge on week 25 was assessed in a plaque assay as previously described. Hancock, G. E. et al. Generation of atypical pulmonary inflammatory responses in BALB/c mice after immunization with the native attachment (G) glycoprotein of respiratory syncytial virus. J Virol 70, 7783-91 (1996). Briefly, the lungs were removed 4 days after challenge, homogenized, clarified, snap frozen, and stored at −70° C. until assayed on Hep-2 cell monolayers.

Serum antibody determinations were made. The geometric mean serum anti-F protein IgM and IgG titers were determined by endpoint ELISA as previously described, Hancock, G. E. et al. Generation of atypical pulmonary inflammatory responses in BALB/c mice after immunization with the native attachment (G) glycoprotein of respiratory syncytial virus. J Virol 70, 7783-91 (1996), using a VersaMax ELISA plate reader (405 nm) and SoftMaxPro software (4 parameter analysis) from Molecular Devices (Sunnyvale, Calif.).

The following parameters and methods for statistical analysis were employed. Significant differences (P<0.05) were determined after log transformation by Tukey-Kramer HSD multiple comparison using JMP® statistical software (SAS Institurte Inc., Cary, N.C.). The data are expressed ±1 SDS.

Results

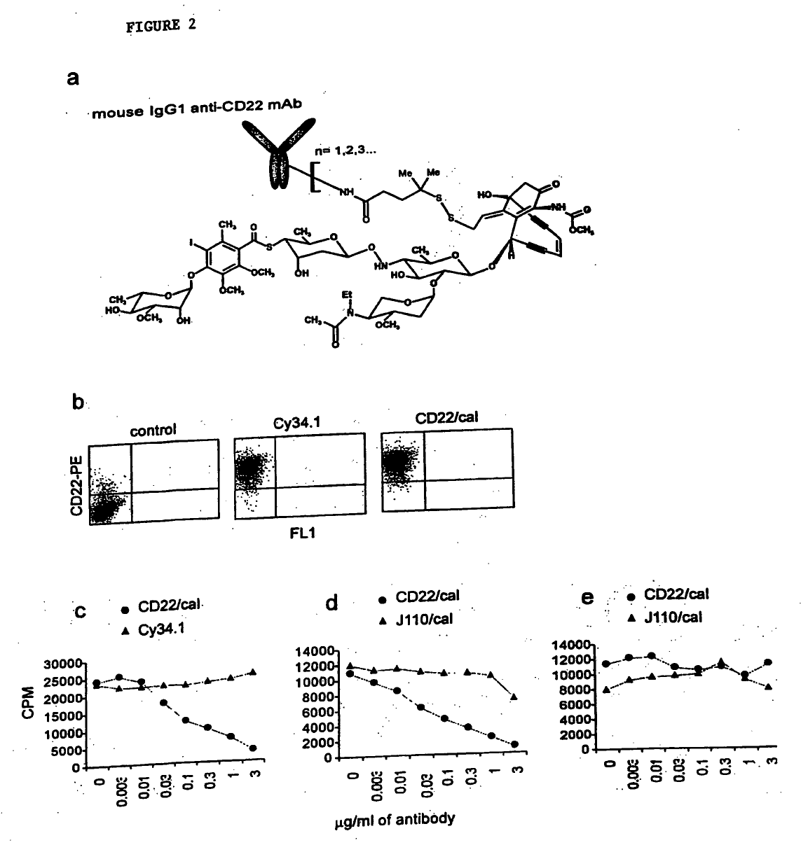

The anti-CD22/calicheamicin (CD22/cal) showed in-vitro B cell specific anti-proliferative effect. The Cy34.1 mAb binded to CD22 expressed on the surface of mouse primary B cells and B cell lines. This antibody was conjugated to calicheamicin (FIG. 2a), a DNA binding antibiotic that induces double stranded DNA breaks in cells after internalization, resulting in cell cycle arrest and apoptosis. Thorson, J. S. et al. Understanding and exploiting nature's chemical arsenal: the past, present and future of calicheamicin research. Curr Pharm Des 6, 1841-79 (2000). Whether biochemical conjugation to calicheamicin (CD22/cal) had any effect on the binding properties of Cy34.1 mAb was examined. Upon staining, both Cy34.1 and CD22/cal bound similarly to CD22 on A20 B cell lymphoma cells (FIG. 2b). To test in-vitro cytotoxicity, purified B cells from male B6 mice were cultured with various concentrations of CD22/cal and proliferation in response to stimulation with LPS was studied after 48 hours of culture. Whereas unconjugated Cy34.1 had no effect, 3 μg/ml CD22/cal completely inhibited B cell proliferation (FIG. 2c). CD22/cal was compared with control antibody conjugated to calicheamcin (J110/cal). The IC50 of CD22/cal was 0.03 μg/ml, whereas the IC50 of the control immunoconjugate J110/cal was 3 μg/ml (FIG. 2d). Thus, CD22/cal was 100-fold more selective relative to the control immunoconjugate. The cytotoxicity of CD22/cal was B cell specific, since the compound had no effect in in vitro T cell proliferation assays. (FIG. 2e).

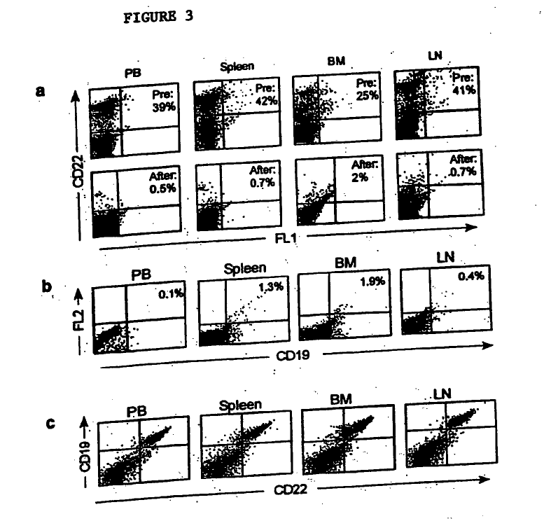

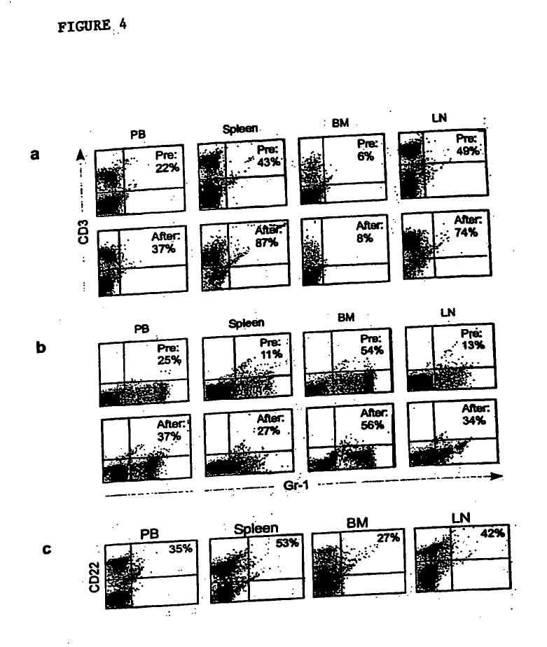

CD22/cal showed in vivo B cell specific cytotoxicity as described below. Based on observations from previous studies in xenograft models, DiJoseph, J. F. et al. Antibody-targeted chemotherapy with CMC-544: a CD22-targeted immunoconjugate of calicheamicin for the treatment of B-lymphoid malignancies. Blood 103, 1807-14 (2004), several dosing schedules were tested to assess the in-vivo cytotoxicity of CD22/cal. The schedule that showed the highest efficacy in all tissues tested (referred as schedule II) consisted of two i.p. injections (160 μg/kg/injection) with CD22/cal, 5 days apart. When B6 mice were treated with schedule II on days 0 and 5, CD22⁺ B cells were almost completely absent from peripheral blood samples as early as day 8 after the first CD22/cal injection (data not shown). Flow cytometry analysis on day 12 further revealed that the percentage of CD22⁺ B cells was decreased from 39% to 0.5% in PB, 42% to 0.7% in spleen, 25% to 2% in BM, and 41% to 0.7% in LN (FIG. 3a). Similar results were obtained when cells were stained with either B220 (data not shown) or anti-CD19 mAb (FIG. 3b). As shown, the same population of B cells in naïve B6 mice expresses both CD22 and CD19 (FIG. 3c). Interestingly, when less efficacious dosing schedules were used, the level of B cell depletion was reproducibly higher in the peripheral blood and lymph nodes as compared to bone marrow and spleen (data not shown). Collectively, these flow cytometric results demonstrate that a protocol consisting of two i.p. injections with 160 μg/CD22/cal/kg, 5 days apart, has very potent cytotoxic activity against B cells in-vivo.

The in-vivo cytotoxicity of the CD22/cal immunoconjugate against T cells and cells of myeloid lineage was also examined by flow cytometry using mAb specific for CD3 (T cell) and Gr-1 (myeloid). On day 12, the percentages of CD3+ and Gr-1+ cells were increased in all tissues tested, presumably due to the depletion of CD22+ B cells (FIGS. 4a, b). Similar results were obtained on day 20 (data not shown). By day 30, the percentage of CD22+ cells was increased in the bone marrow and spleen (9-14%, ±2%), but not in peripheral blood and lymph nodes (data not shown). Five days later, the percentages of CD22+ B cells were significantly higher (but below normal levels) in bone marrow and spleen samples, and remained less than <5% in peripheral blood and lymph node samples (data not shown). Of interest was the observation that 5-8% of CD19+ cells in day 30 and 35 bone marrow and spleen samples were negative for CD22+ expression (data not shown). The numbers of CD22+ B cells were within normal ranges in all tissues tested on day 50 of the experiment (FIG. 4c). Collectively, these results demonstrate that the cytotoxicity CD22/cal immunoconjugate is directed against B cells in-vivo. In addition, repopulation of the bone marrow and spleen with B cells begins approximately 30 days after the first CD22/cal injection and is completely reconstituted within 50 days.

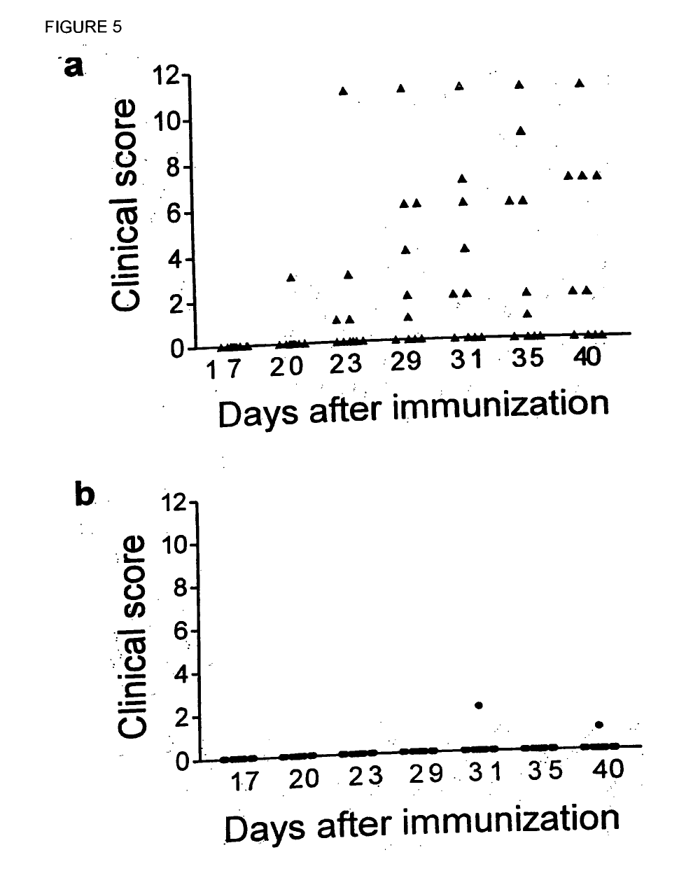

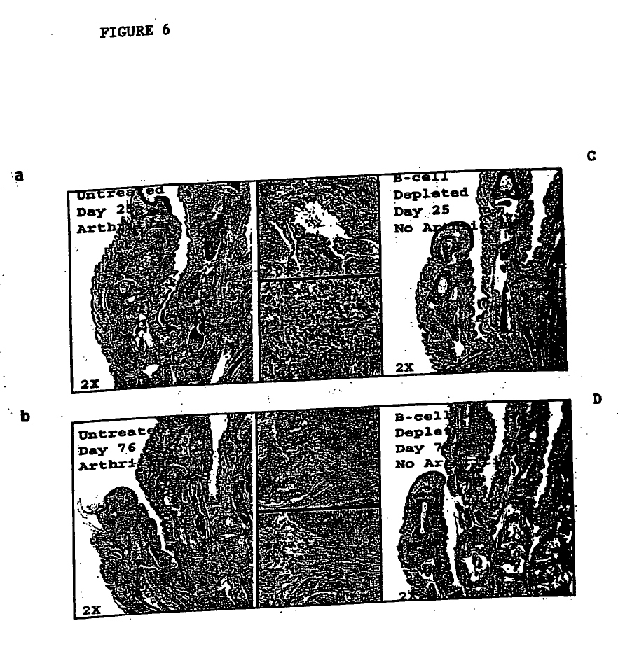

B cell depletion with CD22/cal was observed to inhibit the development of clinical and histological collagen-induced arthritis. One prominent feature of CIA is that susceptibility to disease is restricted to murine strains bearing major histocompatibility complex (MHC) II H-2^{q} or H-2^{r} haplotype, with strains bearing H-2b being amongst the least susceptible strains. Wooley, P. H., Luthra, H. S., Stuart, J. M. & David, C. S. Type II collagen-induced arthritis in mice. I. Major histocompatibility complex (I region) linkage and antibody correlates. J Exp Med 154, 688-700 (1981). Recent reports, however, demonstrated that CIA could be induced in the resistant C57BL/6 (B6) mice if IFN-γ signaling was abolished. Ortmann, R. A. & Shevach, E. M. Susceptibility to collagen-induced arthritis: cytokine-mediated regulation. Clin Immunol 98, 109-18 (2001). The B6 IFN-γ KO CIA model was further characterized by Chu et al. Chu, C. Q., Song, Z., Mayton, L., Wu, B. & Wooley, P. H. IFNgamma deficient C57BL/6 (H-2b) mice develop collagen induced arthritis with predominant usage of T cell receptor Vbeta6 and Vbeta8 in arthritic joints. Ann Rheum Dis 62, 983-90 (2003) who found that 60-80% of mice developed progressive arthritis similar in clinical course to classical CIA observed in DBA/1 mice. Furthermore, B6 IFN-γ KO mice produced significantly higher levels of IgG2b and IgG1 autoantibodies against murine collagen II compared with B6 mice. Note, although direct confirmation of diminished levels of anti-collagen antibodies in this study would have been reassuring, our focus in the CIA model was the clinical and histological scores of the B cell depleted mice. Depletion was verified by flow cytometry analysis of blood samples 6-8 days after the second CD22/cal injection. The percentages of CD22⁺ and CD19⁺ B cells ranged from 0.5 to 2% (data not shown). The paws were evaluated for clinical signs of arthritis using a semiquantitative scoring system (0-4). Whereas 60% of control immunized mice developed arthritis by day 29 (FIG. 5a), injections with CD22/cal immunoconjugate almost completely inhibited the development of clinical arthritis (FIG. 5b). Similar results were obtained in a repeat experiment (data not shown). In both the experiments, treated mice remained free of clinical arthritis beyond day 50, at which time full B cell pool recovery had occurred in peripheral blood samples (data not shown). Paws were collected from two different experiments on days 25 or 75 after immunization for histological evaluation. At day 25, paws from immunized control mice were infiltrated by large numbers of neutrophils and macrophages that surrounded and infiltrated the joints and associated connective tissues consistent with active inflammation (FIG. 6a). In contrast, paws from CD22/cal treated mice had normal joint architecture and were not infiltrated by inflammatory cells consistent with a lack of previous or ongoing arthritis (FIG. 6b). We then compared the day 75 collected paws. At this time point, the percentages of CD22⁺ cells were within normal ranges in PB, LN, and spleen samples (data not shown). Immunized control mice paws had remodeling and destruction of the joints and adjacent structures consistent with chronic arthritis, although the lack of neutrophils and edema indicated that there was no longer active inflammation (FIG. 6c). In contrast, paws from immunized and B cell depleted mice had microscopically normal joints which is consistent with there never having been an arthritic reaction in these paws (FIG. 6d). Collectively, these results demonstrate that B cell depletion with CD22/cal immunoconjugate inhibits the development of collagen-induced arthritis that persists in efficacy upon recovery of the CD22⁺ B cell pool.

B cell depletion with CD22/cal does not affect antibody responses against the F protein of RSV

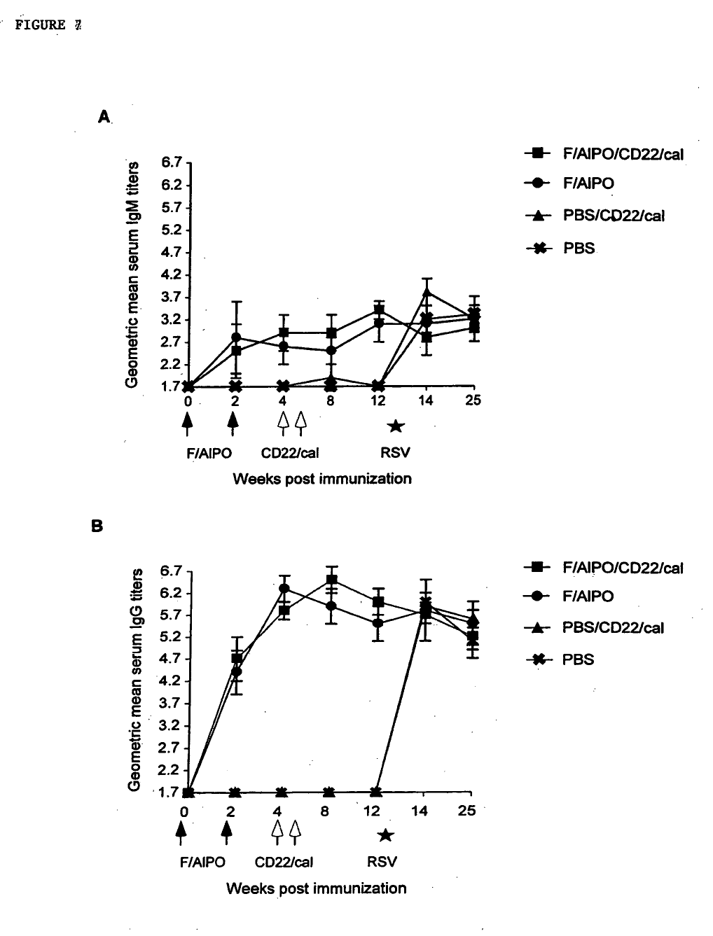

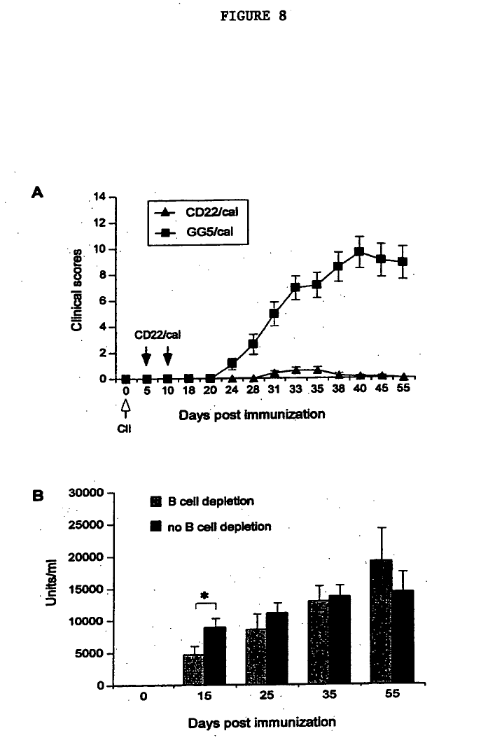

The effects of B cell depletion on serum anti-F protein antibody titers were studied in a murine RSV vaccination model using the protocol depicted in Table 2. Flow cytometry analysis of peripheral blood samples verified B cell depletion in immune mice (Table 3), whereas CD3⁺ and Gr-1⁺ cells were unaffected (data not shown). Mice vaccinated with F antigen prior to B cell depletion had robust IgM and IgG responses to F protein, exhibiting no differences in serum IgM (FIG. 7a) and IgG (FIG. 7b) titers as compared with control (PBS) mice. In addition, infection of B cell depleted naïve mice with the A2 strain of RSV on week 12 (8 weeks after B cell depletion) resulted in comparable levels of anti-F protein IgM (FIG. 7a) and IgG (FIG. 7b) serum titers, as in control (PBS) naïve mice. This suggests that the B cells that have reconstituted by the time of challenge are fully functional and capable of making normal antibody responses. Furthermore, infection of mice with the A2 strain of RSV resulted in comparable anti-F protein IgM and IgG responses in the serum at weeks 14 and 25 (FIGS. 7a, b), indicating that the memory B cell pool for protein F was not affected by treatment with CD22/cal. Finally, infectious virus was not detected in the lungs of mice 4 days after challenge on week 25 of the experiment (data not shown).

The results of the study demonstrate that a B cell depleting protocol consisting of two in-vivo injections with CD22/cal is efficacious in a CIA model, whereas the same protocol does not have an unfavorable effect on memory responses and clearance of virus after challenge in an RSV model. The study also showed that CD22/cal has B cell specific in vitro and in vivo cytotoxicity, leading to depletion of only CD22⁺, but not CD3⁺ T cells and Gr-1⁺ myeloid cells. Mice that have almost undetectable levels of CD22⁺ B cells in bone marrow and spleen start repopulating these organs around day 30-35 and have complete CD22⁺ B cell pool reconstitution 50 days after CD22/cal injections.

CD22/cal binds to CD22, a member of the Ig superfamily that serves as an adhesion receptor for sialic acid bearing ligands. Tuscano, J. M., Riva, A., Toscano, S. N., Tedder, T. F. & Kehrl, J. H. CD22 cross-linking generates B-cell antigen receptor-independent signals that activate the JNK/SAPK signaling cascade. Blood 94, 1382-92 (1999). Mouse CD22 (mCD22) is detected in the cytoplasm early in B cell development (late pro-B cell stage), is absent from the surface of newly emerging IgM⁺ B cells, present at a low density on the immature B220^{lo} IgM^{hi} B cells, and fully expressed by mature B220^{hi} IgD⁺ B cells of the bone marrow. Symington, F. W., Subbarao, B., Mosier, D. E. & Sprent, J. Lyb-8.2: A new B cell antigen defined and characterized with a monoclonal antibody. Immunogenetics 16, 381-91 (1982). In the periphery, mCD22 is expressed at high levels on all B cell subsets including follicular and marginal zone B cells of the spleen and peritoneal B1 cells. However, a minor subset of immature B cells in the spleen recently derived from bone marrow, expresses low density CD22. Tedder, T. F., Tuscano, J., Sato, S. & Kehrl, J. H. CD22, a B lymphocyte-specific adhesion molecule that regulates antigen receptor signaling. Annu Rev Immunol 15, 481-504 (1997). CD22 is constitutively endocytosed and degraded with a relatively short half-life on the cell surface. Shan, D. & Press, O. W. Constitutive endocytosis and degradation of CD22 by human B cells. J Immunol 154, 4466-75 (1995). Upon binding to anti-CD22 mAb, CD22 is rapidly internalized, Shan, D. & Press, O. W. Constitutive endocytosis and degradation of CD22 by human B cells. J Immunol 154, 4466-75 (1995), and this property makes it a suitable target for calicheamicin mediated B cell cytotoxicity. Indeed, in the study, CD22/cal conferred B cell specific in vitro and in vivo cytotoxicity. The pattern and kinetics of CD22 expression on B cells suggests that B cell depletion with CD22/cal was less effective in the bone marrow and spleen, as compared to peripheral blood and lymph nodes, when schedules with doses lower than 160 μg/kg/injection were used. Given that a rapid turnover of newly emerging IgM⁺ B cells constantly occurs in the bone marrow and the spleen, it is reasonable to assume that the concentrations that can effectively deplete B cells in these two organs are higher than the concentrations needed for the peripheral blood and lymph nodes. Of note, the absence of CD22⁺ cells in the peripheral blood did not mirror the level of depletion in the bone marrow and spleen. This observation is important, particularly in relation to B cell ablative therapies in the clinic, and indicates that caution should be paid when clinical responses in these patients are correlated to the level of B cell depletion, since the evaluation of the latter one is based on analysis of peripheral blood samples.

In a clinical study involving 22 RA patients treated with B cell depletion, peripheral blood B lymphocyte counts fell to undetectable levels in all cases and remained below normal for at least 6 months. Leandro, M. J., Edwards, J. C. & Cambridge, G. Clinical outcome in 22 patients with rheumatoid arthritis treated with B lymphocyte depletion. Ann Rheum Dis 61, 883-8 (2002). In the foregoing mouse studies, CD22⁺ B cells were severely depleted from bone marrow and spleen for about a period of 4 weeks. Evidence of repopulation was observed in both tissues between days 30-35, while B cell numbers remained low in lymph node and blood samples. Prior to depletion the same population of B cells expressed both CD22 and CD19 (FIG. 19c). However, on days 30 and 35, more CD19⁺ cells were detected than CD22⁺ cells. It is unlikely that the explanation may be attributed to CD22/cal related cytotoxicity, or to masking of the CD22 epitope on B cells by unconjugated anti-CD22 antibody, since the period after the last injection exceeded by far the expected half life of a mouse IgG antibody.

The CD22/cal studies in the B6 IFN-γ KO CIA model provide strong evidence for the pathogenic role of B cells in the development of arthritis. This is supported by prior observations, linking B cell function with disease in CIA. Cross-breeding CBA/N xid, Thomas, J. D. et al. Colocalization of X-linked agammaglobulinemia and X-linked immunodeficiency genes. Science 261, 355-8 (1993), mice onto the highly susceptible to CIA DBA/1 mice resulted in a strain that was resistant to induction of CIA and did not develop an antibody response to type II collagen. Jansson, L. & Holmdahl, R. Genes on the X chromosome affect development of collagen-induced arthritis in mice. Clin Exp Immunol 94, 459-65 (1993). In addition, mice lacking B cells due to the deletion of the IgM heavy chain gene (muMT) are resistant to CIA. Svensson, L., Jirholt, J., Holmdahl, R. & Jansson, L. B cell-deficient mice do not develop type II collagen-induced arthritis (CIA). Clin Exp Immunol 111, 521-6 (1998). In these models, however, B cells were either reduced and defective (xid) or completely absent (muMT) at the time of immunization with collagen. The development of the in vivo CD22/cal protocol using B6 IFN-γ KO mice enabled us to evaluate the role of B cell depletion on CIA initiated during priming and effector responses of T and B cells to the injected collagen II. The B6 IFN-γ KO mice not only remained free of clinical and histological signs of arthritis during the CD22⁺ B cell depletion period, but also after complete reconstitution of the CD22⁺ B cell pool, as demonstrated by day 75 paw histology. These data suggest that the CD22/cal immunoconjugate permanently inhibited the generation and expansion of pathogenic B cell clones reactive to self collagen in immunized mice. Alternatively, but not mutually exclusive, CD22/cal may have reduced pathogenic B cells to a level that, even after complete reconstitution of the B cell pool, remained insufficient for the generation of inflammatory mechanisms leading to clinical and/or histological arthritis. In this context, CD22/cal may have eliminated B cells that exhibit diverse functions and display pathogenic characteristics, other than autoantibody production. Duddy, M. E., Alter, A. & Bar-Or, A. Distinct profiles of human B cell effector cytokines: a role in immune regulation? J Immunol 172, 3422-7 (2004). Porakishvili, N. et al. Recent progress in the understanding of B-cell functions in autoimmunity. Scand J Immunol 54, 30-8 (2001). In support of this concept are data from recent trials in predominantly SLE and ITP patients showing that clinical responses to B cell depletion can occur without concomitant changes in autoantibody titers. Martin, F. & Chan, A. C. Pathogenic roles of B cells in human autoimmunity; insights from the clinic. Immunity 20, 517-27 (2004). Thus, it is likely that there are additional mechanisms by which B cell lineage depletion modulates autoimmune diseases. Martin, F. & Chan, A. C. Pathogenic roles of B cells in human autoimmunity; insights from the clinic. Immunity 20, 517-27 (2004). Anolik, J., Sanz, I. & Looney, R. J. B cell depletion therapy in systemic lupus erythematosus. Curr Rheumatol Rep 5, 350-6 (2003). Looney, R. J., Anolik, J. & Sanz, I. B cells as therapeutic targets for rheumatic diseases. Curr Opin Rheumatol 16, 180-5 (2004). In RA patients with lymphoid aggregates within the synovium, B cells may function as antigen presenting cells and provide costimulatory signals that promote expansion of effector T cells. B cells within the synovium may also secrete proinflammatory cytokines and contribute to inflammation. Weyand, C. M. & Goronzy, J. J. Ectopic germinal center formation in rheumatoid synovitis. Ann NY Acad Sci 987, 140-9 (2003). Duddy, M. E., Alter, A. & Bar-Or, A. Distinct profiles of human B cell effector cytokines: a role in immune regulation? J Immunol 172, 3422-7 (2004). Pistoia, V. Production of cytokines by human B cells in health and disease. Immunol Today 18, 343-50 (1997).

The effect of CD22/cal was also examined in the B6 mice immunized with the F protein of RSV. The primary purpose of these studies was to evaluate whether prior B cell depletion with CD22/cal immunoconjugate would adversely affect the development of anti-F protein IgM and IgG responses in naïve and F protein-educated mice. Our interest was motivated by concerns that B cell ablation might significantly diminish, if not abolish, most of the B cell pool, including previously educated “memory” B cells, and thus cause hypogammaglobulinemia and humoral immunodeficiency. The results herein demonstrate that serum immunoglobulin levels remained within normal ranges in mice that were treated with the CD22/cal protocol, and that these mice were able to exhibit normal Ig responses and clearance of virus after challenge. This observation is in agreement with data obtained from a clinical trial with anti-CD20, showing that a significant drop in autoantibodies could be achieved without a concomitant loss in specific IgG antibodies against tetanus toxoid and pneumococcal capsular polysaccharides. Cambridge, G. et al. Serologic changes following B lymphocyte depletion therapy for rheumatoid arthritis. Arthritis Rheum 48, 2146-54 (2003). This selective effect observed in the clinic can be extrapolated to the observations in the CIA and RSV models, showing that CD22/cal is effective in the CIA model of autoimmunity, in the absence of an unfavorable effect in the RSV vaccination model. As postulated for B cell depleted RA patients, Cambridge, G. et al. Serologic changes following B lymphocyte depletion therapy for rheumatoid arthritis. Arthritis Rheum 48, 2146-54 (2003), Manz, R. A. & Radbruch, A. Plasma cells for a lifetime? Eur J Immunol 32, 923-7 (2002), the B cell clones responsible for production of antiviral antibodies may reside in the spleen and experience slow turnover into CD22 negative plasma cells, whereas autoantibodies may be more dependent on the constant generation of new plasma cells from CD22-positive B lymphocytes. Also, collagen-reactive B cell clones are possibly in a more dynamic state because of constant generation, and while entering the circulation in larger numbers than normal B cells, they inevitably become more susceptible to CD22/cal. Most importantly, depletion of B cells in the RSV model allowed for the preservation of humoral immunity to preexisting memory responses, and allowed the generation of humoral immunity to new antigens upon reconstitution of the B cell compartment.

TABLE 2
The protocol for immunization and treatment of C57BI/6
mice respectively with F/AIPO and CD22/cal

| | Week | | | | | | | | | |
|---|---|---|---|---|---|---|---|---|---|---|
| Group | 0 | 2 | 4 | 4/5 d | 6 | 8 | 12 | 12/4 d | 14/4 d | 25 |
| #1 | B/V | B/V | B/CD22/cal | CD22/cal | B | B | B | C | B | B/C |
| #2 | B/V | B/V | B/PBS | PBS | B | B | B | C | B | B/C |
| #3 | B | — | B/CD22/cal | CD22/cal | B | B | B | C | B | B/C |
| #4 | B | — | B/PBS | PBS | B | B | B | C | B | B/C |

TABLE 3
B220⁺ cells in the peripheral blood samples
of C57BL/6 mice administered CD22/cal or PBS.
Relative % whole blood leukocytes staining for B220^{a}

| Vaccine | CD22/cal | Pre | Post |
|---|---|---|---|
| F/AIPO | CD22/cal | 34.4 ± 6.3 | 2.0 ± 0.7 |
| F/AIPO | PBS | 44.0 ± 1.6 | 38.2 ± 6.1 |
| PBS | CD22/cal | 38.6 ± 4.1 | 2.5 ± 0.6 |
| PBS | PBS | 40.4 ± 1.6 | 44.9 ± 2.3 |

^{a}Groups of 10 C56BI/6 mice were vaccinated on weeks 0 and 2 with F/AIPO. Immediately before (Pre) and 9 days (Post) after the second administration of CD22/cal peripheral blood leukocytes from vaccinated and naïve mice were analyzed by flow cytometry for cell surface marker B220

TABLE 4
The serum anti-F protein IgM titers of mice depleted of peripheral
blood B cells with cytotoxic drug/B cell depleting agent conjugate.
Geometric Mean Anti-F protein IgM Titers (Log₁₀)^{a}

| Vaccine | Conjugate | WK 0 | WK 2 | WK 4 | WK 8 | WK 12 | WK 14 | WK 25 |
|---|---|---|---|---|---|---|---|---|
| F/AIPO | Conjugate | <1.7 | 2.5 ± 0.6 | 2.9 ± 0.4 | 2.9 ± 0.4 | 3.4 ± 0.2 | 2.8 ± 0.4 | 3.0 ± 0.3 |
| F/AIPO | PBS | <1.7 | 2.8 ± 0.8 | 2.6 ± 0.4 | 2.5 ± 0.3 | 3.1 ± 0.4 | 3.1 ± 0.4 | 3.2 ± 0.2 |
| PBS | Conjugate | <1.7 | <1.7 | <1.7 | 1.9 ± 0.3 | <1.7 | 3.8 ± 0.3 | 3.2 ± 0.3 |
| PBS | PBS | <1.7 | <1.7 | <1.7 | <1.7 | <1.7 | 3.2 ± 0.3 | 3.3 ± 0.4 |

^{a}The geometric mean endpoint titers were determined by ELISA on serum samples of 5 mice per group. Significant differences between the groups were not observed.

TABLE 5
The serum anti-F protein IgG titers of mice depleted of B cells with CD22/cal^{a}

| Vaccine | CD22/cal | WK 0 | WK 2 | WK 4 | WK 8 | WK 12 | WK 14 | WK 25 |
|---|---|---|---|---|---|---|---|---|
| F/AIPO | CD22/cal | <1.7 | 4.7 ± 0.5 | 5.8 ± 0.2 | 6.5 ± 0.3 | 6.0 ± 0.3 | 5.7 ± 0.2 | 5.2 ± 0.3 |
| F/AIPO | PBS | <1.7 | 4.4 ± 0.5 | 6.3 ± 0.3 | 5.9 ± 0.4 | 5.5 ± 0.4 | 5.8 ± 0.7 | 5.5 ± 0.5 |
| PBS | CD22/cal | <1.7 | <1.7 | <1.7 | <1.7 | <1.7 | 5.9 ± 0.08 | 5.6 ± 0.2 |
| PBS | PBS | <1.7 | <1.7 | <1.7 | <1.7 | <1.7 | 6.0 ± 0.2 | 5.1 ± 0.4 |

^{a}The geometric mean endpoint titers were determined by ELISA on serum samples of 5 mice per group. Significant differences between the groups were not observed.

All references and patents cited above are incorporated herein by reference. Numerous modifications and variations of the present inventions are included in the above-identified specification and are expected to be obvious to one of skill in the art. Such modifications and alterations to the conjugation process, the conjugates made by the process, and to the compositions/formulations comprising conjugates are believed to be encompassed within the scope of the claims.

### BIBLIOGRAPHY

U.S. Pat. No. 4,671,958
U.S. Pat. No. 4,970,198
U.S. Pat. No. 5,037,651
U.S. Pat. No. 5,053,394
U.S. Pat. No. 5,079,233
U.S. Pat. No. 5,712,374
U.S. Pat. No. 5,714,586
U.S. Pat. No. 5,773,001
U.S. Pat. No. 5,877,296
U.S. Pat. No. 5,877,296
PCT publication No. WO 91/09967
PCT publication No. WO 98/20734
Andrews R., Singer J., Bernstein I., J. Exp. Med.; 169:1721-1731 (1989).
Anolik, J., I. Sanz, et al. (2003). “B cell depletion therapy in systemic lupus erythematosus.” Curr Rheumatol Rep 5(5): 350-6.
Benoist, C. and D. Mathis (2000). “A revival of the B cell paradigm for rheumatoid arthritis pathogenesis?” Arthritis Res 2(2): 90-4.
Berek, C. and A. E. Schroder (1997). “A germinal center-like reaction in the nonlymphoid tissue of the synovial membrane.” Ann NY Acad Sci 815: 211-7.
Berek, C. and H. J. Kim (1997). “B-cell activation and development within chronically inflamed synovium in rheumatoid and reactive arthritis.” Semin Immunol 9(4): 261-8.
Berek, C. and M. Ziegner (1993). “The maturation of the immune response.” Immunol Today 14(8): 400-4.
Berger M., Leopold L., Dowell J., Korth-Bradley J., Sherman M., Invest. New Drugs; 20: 395-406 (2002).
Bross P. F., Beitz J., Chen G., Chen X. H., Duffy E., Keiffer-Bross P., Beitz J., Chen G., Chen X., Duffy E., Kieffer L., Roy S., Sridhara R., Rahman A., Williams G., Pazdur R., Clin. Cancer Res., 7:1490-1496 (2001).
Cambridge, G., M. J. Leandro, et al. (2003). “Serologic changes following B lymphocyte depletion therapy for rheumatoid arthritis.” Arthritis Rheum 48(8): 2146-54.
Chu, C. Q., Z. Song, et al. (2003). “IFNgamma deficient C57BL/6 (H-2b) mice develop collagen induced arthritis with predominant usage of T cell receptor Vbeta6 and Vbeta8 in arthritic joints.” Ann Rheum Dis 62(10): 983-90.
Crameri et al., Nature, 391, 288-291, (1998).
Crocker P. R. and Varki A. Siglecs, Trends in Immunol.; 22:337-342 (2001).
Damle, N. K. and P. Frost (2003). “Antibody-targeted chemotherapy with immunoconjugates of calicheamicin.” Curr Opin Pharmacol 3(4): 386-90.
DiJoseph, J. F., D. C. Armellino, et al. (2004). “Antibody-targeted chemotherapy with CMC-544: a CD22-targeted immunoconjugate of calicheamicin for the treatment of B-lymphoid malignancies.” Blood 103(5): 1807-14.
Dowell J. A., Korth-Bradley J., Liu H., King S. P., Berger M. S., J. Clin. Pharmacol.; 41:1206-1214 (2001).
Dubowchik G. and Walker M., Pharmacol. & Therapeutics, 83:67-123 (1999).
Duddy, M. E., A. Alter, et al. (2004). “Distinct profiles of human B cell effector cytokines: a role in immune regulation?” J Immunol 172(6): 3422-7.
Edward Chu and Vincent T. DeVita, Physician's Cancer Chemotherapy Drug Manual, Publishers: Jones and Bartlett (2002).
Feldmann, M., F. M. Brennan, et al. (1996). “Role of cytokines in rheumatoid arthritis.” Annu Rev Immunol 14: 397-440.
G. Kohler and Milstein, C., Nature, 256:495 (1975).
Gause, A. and C. Berek (2001). “Role of B cells in the pathogenesis of rheumatoid arthritis: potential implications for treatment.” BioDrugs 15(2): 73-9.
Gibaldi M., Perrier D., Pharmacokinetics, 2^{nd} ed., Marcel-Dekker Inc., NY (1982).
H. McConnell and Hoess, J., J. Mol. Biol. 250:460 (1995).
Hamann P., Hinman L., Beyer C., Kindh D., Upeslacis J., Flowers D., Bernstein I., Choice of Linker. Bioconj. Chem.; 13:40-46 (2002).
Hamann P., Hinman L., Hollander I., Beyer C., Lindh D., Holcomb R., Hallet W., Tsou H., peslacis J., Shochat D., et al., Bioconj. Chem.; 13:47-58 (2002).
Hamann, P. R., L. M. Hinman, et al. (2002). “An anti-CD33 antibody-calicheamicin conjugate for treatment of acute myeloid leukemia. Choice of linker.” Bioconjug Chem 13(1): 40-6.
Hancock, G. E., D. J. Speelman, et al. (1996). “Generation of atypical pulmonary inflammatory responses in BALB/c mice after immunization with the native attachment (G) glycoprotein of respiratory syncytial virus.” J Virol 70(11): 7783-91.
Hancock, G. E., P. W. Tebbey, et al. (2003). “Immune responses to the nonglycosylated ectodomain of respiratory syncytial virus attachment glycoprotein mediate pulmonary eosinophilia in inbred strains of mice with different MHC haplotypes.” J Med Virol 70(2): 301-8.
Hanna R., Ong G. L., Mattes M. J., Cancer Res.; 56:3062-3068 (1996).
Hinman L. M., Hamann P. R., Wallace R., Menendez A. T., Durr F. E., Upeslacis J. Cancer Res. 53:3336-3342 (1993).
Hursey M., Newton D. L., Hansen H. J., Ruby D., Goldenberg D. M., Rybak S. M., Leukemia and Lymphoma; 43:953-959 (2002).
I. D. Bernstein et al., J. Clin. Invest. 79:1153 (1987).
I. D. Bernstein et al., J. Immunol. 128:867-881 (1992).
J. Tramontano; et al., J. Mol. Recognit. 7:9 (1994).
Jacob, J., G. Kelsoe, et al. (1991). “Intraclonal generation of antibody mutants in germinal centres.” Nature 354(6352): 389-92.
Jansson, L. and R. Holmdahl (1993). “Genes on the X chromosome affect development of collagen-induced arthritis in mice.” Clin Exp Immunol 94(3): 459-65.
Kabat et al. Sequencing. of Proteins of Immunological. Interest, 1:310-334 (1994).
Kim, H. J. and C. Berek (2000). “B cells in rheumatoid arthritis.” Arthritis Res 2(2): 126-31.
Korganow, A. S., H. Ji, et al. (1999). “From systemic T cell self-reactivity to organ-specific autoimmune disease via immunoglobulins.” Immunity 10(4): 451-61.
Kouskoff, V., A. S. Korganow, et al. (1996). “Organ-specific disease provoked by systemic autoimmunity.” Cell 87(5): 811-22.
Kreitman R. J., Current Pharmaceut. Biotech.; 2:313-325 (2001).
Kreitman R. J., Curr. Opin. Mol. Ther.; 5:44-551 (2003).
Kreitman R. J., Wilson W. H., Bergeron K., Raggio M., Stetler-Stevenson M., Fitzgerald D. J., Pastan I., N. Enql. J. Med.; 345:241-247 (2001).
Ku and Schultz, Proc. Natl. Acad. Sci., USA 92:6552 (1995).
Larson R., Boogaerts M., Estey E., Karanes C., Stadtmauer E., Sievers E., Mineur P., Bennett J., Berger M., Eten C. et al. Leukemia; 16:1627-1636 (2002).
Leandro, M. J., J. C. Edwards, et al. (2002). “An open study of B lymphocyte depletion in systemic lupus erythematosus.” Arthritis Rheum 46(10): 2673-7.
Leandro, M. J., J. C. Edwards, et al. (2002). “Clinical outcome in 22 patients with rheumatoid arthritis treated with B lymphocyte depletion.” Ann Rheum Dis 61(10): 883-8.
Lee M., Dunne T., Chang C., Siegal M., Morton G., Ellestad G., McGahren W., Borders D., J. Am. Chem. Soc. 1992; 114:985-987 (1992).
Leonard J. P. and Link B. K., Sem. Oncol. 29:81-86 (2002).
Looney, R. J., J. Anolik, et al. (2004). “B cells as therapeutic targets for rheumatic diseases.” Curr Opin Rheumatol 16(3): 180-5.
Low et al., J. Mol. Biol., 250, 359-368 (1996).
Maccioni, M., G. Zeder-Lutz, et al. (2002). “Arthritogenic monoclonal antibodies from K/BxN mice.” J Exp Med 195(8): 1071-7.
Markand et al., Biochemistry 35:8045 (1996).
Markand et al., Biochemistry 35:8098 (1996).
Marrack, P., J. Kappler, et al. (2001). “Autoimmune disease: why and where it occurs.” Nat Med 7(8): 899-905.
Martin, F. and A. C. Chan (2004). “Pathogenic roles of B cells in human autoimmunity; insights from the clinic.” Immunity 20(5): 517-27.
Matsumoto, I., A. Staub, et al. (1999). “Arthritis provoked by linked T and B cell recognition of a glycolytic enzyme.” Science 286(5445): 1732-5.
Moyron-Quiroz J. E., Partida-Sanchez S., Donis-Hernandez R., Sandoval-Montes C. and Santos-Argumedo L., Scand. J. Immunol.; 55:343-351 (2002).
Nitschke L., Floyd H., and Crocker P. R., Scand. J. Immunol.; 53:227-234 (2001).
Nord et al., Nat. Biotechnol. 15:772 (1997).
Nord et al., Protein Eng. 8:601 (1995).
Ortmann, R. A. and E. M. Shevach (2001). “Susceptibility to collagen-induced arthritis: cytokine-mediated regulation.” Clin Immunol 98(1): 109-18.
Pastan I., Kreitman R. J., Current Opin. Investig. Drugs, 3(7):1089-1091 (2002).
Patel, D. D. (2002). “B cell-ablative therapy for the treatment of autoimmune diseases.” Arthritis Rheum 46(8): 1984-5.
Patten et al., Curr. Opin. Biotechnol., 8, 724-733, (1997).
Rottgen and Collins, Gene 164:243 (1995).
Saito, K., M. Nawata, et al. (2003). “Successful treatment with anti-CD20 monoclonal antibody (rituximab) of life-threatening refractory systemic lupus erythematosus with renal and central nervous system involvement.” Lupus 12(10): 798-800.
Schindler J., Sausville E., Messmann R., Uhr J. W. & Vitetta, E. S., Clin. Cancer Res., 7, 255-258 (2001).
Shan D. and Press O.W., J. Immunol. 1995; 154:4466-4475 (1995).
Shan, D. and O.W. Press (1995). “Constitutive endocytosis and degradation of CD22 by human B cells.” J Immunol 154(9): 4466-75.
Sievers E., Larson R., Stadmauer E., Estey E., Lowenberg B., Dombret H., Karanes C., Theobald M., Bennet J., Sherman M., et al., J. Clin. Oncol.; 19:3244-3254 (2001).
Stasi, R., A. Pagano, et al. (2001). “Rituximab chimeric anti-CD20 monoclonal antibody treatment for adults with chronic idiopathic thrombocytopenic purpura.” Blood 98(4): 952-7.
Svensson, L., J. Jirholt, et al. (1998). “B cell-deficient mice do not develop type I collagen-induced arthritis (CIA).” Clin Exp Immunol 111(3): 521-6.
Symington, F. W., B. Subbarao, et al. (1982). “Lyb-8.2: A new B cell antigen defined and characterized with a monoclonal antibody.” Immunogenetics 16(5): 381-91.
Szczepanski, L., J. Szechinski, et al. (2003). Safety Data From 48 weeks Follow-Up of a Randomised Controlled Trial of Rituximab in Patients with Rheumatoid Arthritis. ACR, Arthritis & Rheumatism.
T. G. Hose and Blair, A. H. CRC Critical Rev. Drug Carrier Systems 3:263 (1987).
Takemura, S., P. A. Klimiuk, et al. (2001). “T cell activation in rheumatoid synovium is B cell dependent.” J Immunol 167(8): 4710-8.
Tedder T. F., Tuscano J., Sato S., Kehrl J. H., Ann. Rev. Immunol.; 15:481-504 (1997).
Tedder, T. F., J. Tuscano, et al. (1997). “CD22, a B lymphocyte-specific adhesion molecule that regulates antigen receptor signaling.” Annu Rev Immunol 15: 481-504.
Thomas, J. D., P. Sideras, et al. (1993). “Colocalization of X-linked agammaglobulinemia and X-linked immunodeficiency genes.” Science 261(5119): 355-8.
Thompson et al., J. Mol. Biol., 256, 77-88, (1996).
Thorson J., Sievers E., Ahlert J., Shepard E., Whitwam R., Onwueme K., Ruppen M., Current Pharmaceut. Design; 6:1841-1879 (2000).
Trail P and Bianchi A., Current Opin. Immunol., 11:584-588, (1999).
Tuscano, J. M., A. Riva, et al. (1999). “CD22 cross-linking generates B-cell antigen receptor-independent signals that activate the JNK/SAPK signaling cascade.” Blood 94(4): 1382-92.
Van Horssen P. J., Preijers, F. W., Van Oosterhout, Y. V., Eling W. M. and De Witte, T., Leukemia & Lymphoma; 39(5-6):591-599 (2000).
Vincent T. DeVita, Samuelo Hellman, Steven A. Rosenberg, Eds., Cancer Principles and Practice of Oncology, 6^{th} Edition, Publishers: Lippincott, Williams and Wilkins (2001).
Wang et al, J. Biol. Chem., 270:12250 (1995).
Weyand, C. M. and J. J. Goronzy (2003). “Ectopic germinal center formation in rheumatoid synovitis.” Ann NY Acad Sci 987: 140-9.
Wooley, P. H., H. S. Luthra, et al. (1981). “Type II collagen-induced arthritis in mice. I. Major histocompatibility complex (I region) linkage and antibody correlates.” J Exp Med 154(3): 688-700.
Yang et al., J. Mol. Biol., 254, 392-403 (1995).
Zein N., Sinha A., McGahren W., Ellestad G., Science; 240:1198-1201 (1988).

The invention now being fully described, it will be apparent to one of ordinary skill in the art that many changes and modifications can be made thereto without departing from the spirit or scope of the invention as set forth herein. The foregoing describes the preferred embodiments of the present invention along with a number of possible alternatives. These embodiments, however, are merely for example and the invention is not restricted thereto.

1. A method for treating an autoimmune disease in a subject comprising:
administering to the subject a therapeutically effective amount of: (a) a B cell depleting agent; and (b) at least one anti-cytokine agent.

2. The method of claim 1, wherein the B cell depleting agent is an antibody.

3. The method of claim 2, wherein the antibody is selected from the group consisting of a monoclonal antibody, a chimeric antibody, a human antibody, a humanized antibody, a human antibody produced in a transgenic animal, a single chain antibody, a Fab fragment and a F(ab)2 fragment.

4. The method of claim 2, wherein the antibody is selected from the group consisting of anti-CD19, anti-CD20, and anti-CD22 antibodies.

5. The method of claim 2, wherein the antibody is a humanized antibody directed against the cell surface antigen CD22.

6. The method of claim 5, wherein the humanized anti-CD22 antibody is a CDR-grafted antibody, and comprises a light chain variable region 5/44-gL1 (SEQ ID NO:19), and a heavy chain variable region 5/44-gH7 (SEQ ID NO:27).

7. The method of claim 5, wherein the humanized anti-CD22 antibody is a CDR-grafted antibody comprising a light chain having a sequence set forth in SEQ ID NO: 28.

8. The method of claim 5, wherein the humanized anti-CD22 antibody is a CDR-grafted antibody comprising a heavy chain having a sequence set forth in SEQ ID NO:30.

9. The method of claim 5, wherein the humanized anti-CD22 antibody is a CDR-grafted antibody comprising a light chain having a sequence set forth in SEQ ID NO: 28 and a heavy chain having a sequence set forth in SEQ ID NO: 30.

10. The method of claim 5, wherein the humanized anti-CD22 antibody is a CDR-grafted antibody that is a variant antibody obtained by an affinity maturation protocol and has increased specificity for human CD22.

11. The method of claim 1, wherein the B cell depleting agent is conjugated to a cytotoxic drug or cytotoxic drug derivative to form a cytotoxic drug/B cell depleting agent conjugate.

12. The method of claim 11, wherein the cytotoxic drug is calicheamicin.

13. The method of claim 12, wherein the calicheamicin is gamma calicheamicin or N-acetyl calicheamicin.

14. The method of claim 11, wherein the cytotoxic drug is functionalized with 3-mercapto-3-methyl butanoyl hydrazide.

15. The method of claim 11, wherein the cytotoxic drug is conjugated to the bioactive agent through a linker.

16. The method of claim 15, wherein the linker is a hydrolyzable linker that is capable of releasing the cytotoxic drug from the conjugate after binding and entry into target cells.

17. The method of claim 16, wherein the hydrolyzable linker is 4-(4-acetylphenoxy) butanoic acid (AcBut).

18. The method of claim 11, wherein the cytotoxic drug is a monomeric calicheamicin derivative and the bioactive agent is an anti-CD22-antibody to form a monomeric calicheamicin derivative/anti-CD22 antibody conjugate.

19. The method of claim 18, wherein the monomeric calicheamicin derivative/anti-CD22 antibody conjugate is CMC-544.

20. The method of claim 11, wherein the cytotoxic drug/B cell depleting agent conjugate comprises the formula:
Pr(—X—W)m
wherein:
Pr is a bioactive agent,
X is a linker that comprises a product of any reactive group that can react with a bioactive agent,
W is a cytotoxic drug;
m is the average loading for a purified conjugation product such that the cytotoxic drug constitutes 7-9% of the conjugate by weight; and
(—X—W)m is a cytotoxic drug derivative.

21. The method of claim 11, wherein the cytotoxic drug/B cell depleting agent conjugate is prepared by a method comprising:
(1) adding the cytotoxic drug derivative to the bioactive agent wherein the cytotoxic drug derivative is 4.5-11% by weight of the bioactive agent;
(2) incubating the cytotoxic drug derivative and a bioactive agent in a non-nucleophilic, protein-compatible, buffered solution having a pH in the range from about 7 to 9 to produce a monomeric cytotoxic drug/B cell depleting agent conjugate, wherein the solution further comprises (a) an organic cosolvent, and (b) an additive comprising at least one C₆-C₁₈ carboxylic acid or its salt, and wherein the incubation is conducted at a temperature ranging from about 30° C. to about 35° C. for a period of time ranging from about 15 minutes to 24 hours; and
(3) subjecting the conjugate produced in step (2) to a chromatographic separation process to separate monomeric cytotoxic drug derivative/bioactive agent conjugates with a loading in the range of 4-10% by weight cytotoxic drug and with low conjugated fraction (LCF) below 10 percent from unconjugated bioactive agent, cytotoxic drug derivative, and aggregated conjugates.

22. The method of claim 1, wherein the anti-cytokine agent is an anti-TNF agent.

23. The method of claim 22, wherein the anti-TNF agent is etanercept.

24. The method of claim 1, wherein the autoimmune disease is rheumatoid arthritis (RA), Systemic Lupus (SLE), an immune cytopenia, an immune vasculitis or combinations thereof.

25. The method of claim 1, wherein the autoimmune disease is collagen-induced arthritis (CIA) in an experimental animal model.

26. A method for treating an autoimmune disease in a subject comprising:
administering to the subject a therapeutically effective amount of a monomeric cytotoxic drug/B cell depleting agent conjugate with reduced low conjugated fraction (LCF) having the formula,
Pr(—X—W)m
wherein:
Pr is a B cell depleting agent,
X is a linker that comprises a product of any reactive group that can react with a B cell depleting agent,
W is a cytotoxic drug;
m is the average loading for a purified conjugation product such that the cytotoxic drug constitutes 7-9% of the conjugate by weight; and
(—X—W)m is a cytotoxic drug derivative.

27. The method of claim 26, wherein the monomeric cytotoxic drug/B cell depleting agent conjugate is prepared by a method comprising:
adding the cytotoxic drug derivative to the B cell depleting agent wherein the cytotoxic drug derivative is 4.5-11% by weight of the B cell depleting agent;
incubating the cytotoxic drug derivative and a B cell depleting agent in a non-nucleophilic, protein-compatible, buffered solution having a pH in the range from about 7 to 9 to produce a monomeric cytotoxic drug/B cell depleting agent conjugate, wherein the solution further comprises (a) an organic cosolvent, and (b) an additive comprising at least one C₆-C₁₈ carboxylic acid or its salt, and wherein the incubation is conducted at a temperature ranging from about 30° C. to about 35° C. for a period of time ranging from about 15 minutes to 24 hours; and
subjecting the conjugate produced in step (2) to a chromatographic separation process to separate monomeric cytotoxic drug derivative/B cell depleting agent conjugates with a loading in the range of 4-10% by weight cytotoxic drug and with low conjugated fraction (LCF) below 10 percent from unconjugated B cell depleting agent, cytotoxic drug derivative, and aggregated conjugates.

28. The method of claim 26, wherein the B cell depleting agent is selected from a group consisting of hormones, growth factors, antibodies, antibody fragments, antibody mimics, and their genetically or enzymatically engineered counterparts.

29. The method of claim 26, wherein the B cell depleting agent is an antibody.

30. The method of claim 29, wherein the antibody is selected from a group consisting of a monoclonal antibody, a chimeric antibody, a human antibody, a humanized antibody, a human antibody produced in a transgenic animal, a single chain antibody, a Fab fragment and a F(ab)2 fragment.

31. The method of claim 30, wherein the humanized antibody is directed against the cell surface antigen CD22.

32. The method of claim 31, wherein the humanized anti-CD22 antibody is a CDR-grafted antibody, and comprises a light chain variable region 5/44-gL1 (SEQ ID NO:19), and a heavy chain variable region 5/44-gH7 (SEQ ID NO:27).

33. The method of claim 31, wherein the humanized anti-CD22 antibody is a CDR-grafted antibody comprising a light chain having a sequence set forth in SEQ ID NO: 28.

34. The method of claim 31, wherein the humanized anti-CD22 antibody is a CDR-grafted antibody comprising a heavy chain having a sequence set forth in SEQ ID NO:30.

35. The method of claim 31, wherein the humanized anti-CD22 antibody is a CDR-grafted antibody comprising a light chain having a sequence set forth in SEQ ID NO: 28 and a heavy chain having a sequence set forth in SEQ ID NO: 30.

36. The method of claim 31, wherein the humanized anti-CD22 antibody is a CDR-grafted antibody that is a variant antibody obtained by an affinity maturation protocol and has increased specificity for human CD22.

37. The method of claim 26, wherein the additive of step (2) (b) is octanoic acid or its salt.

38. The method of claim 26, wherein the additive of step (2) (b) is decanoic acid or its salt.

39. The method of claim 26, wherein the chromatographic separation process of step (3) is size exclusion chromatography (SEC).

40. The method of claim 26, wherein the chromatographic separation process of step (3) is HPLC, FPLC or Sephacryl S-200 chromatography.

41. The method of claim 26, wherein the chromatographic separation process of step (3) is hydrophobic interaction chromatography (HIC).

42. The method of claim 41, wherein the hydrophobic interaction chromatography (HIC) is carried out using Phenyl Sepharose 6 Fast Flow chromatographic medium, Butyl Sepharose 4 Fast Flow chromatographic medium, Octyl Sepharose 4 Fast Flow chromatographic medium, Toyopearl Ether-650M chromatographic medium, Macro-Prep methyl HIC medium or Macro-Prep t-Butyl HIC medium.

43. The method of claim 41, wherein the hydrophobic interaction chromatography (HIC) is carried out using Butyl Sepharose 4 Fast Flow chromatographic medium.

44. The method of claim 26, additionally comprising administering to the subject an anti-cytokine agent.

45. The method of claim 44, wherein the anti-cytokine agent is an anti-TNF agent.

46. A method of treating an autoimmune disease in a subject comprising: administering to the subject with the autoimmune disease a therapeutically effective amount of a monomeric calicheamicin derivative/anti-CD22 antibody conjugate having the formula,
Pr(—X—S—S—W)ₘ
wherein:
Pr is an anti-CD22 antibody;
X is a hydrolyzable linker that comprises a product of any reactive group that can react with an antibody;
W is a calicheamicin radical;
m is the average loading for a purified conjugation product such that the calicheamicin constitutes 4-10% of the conjugate by weight; and
(—X—S—S—W)m is a calicheamicin derivative.

47. The method of claim 46, wherein the anti-CD22 antibody has specificity for human CD22 and comprises a heavy chain wherein the variable domain comprises a CDR having at least one of the sequences given as H1 in FIG. 1 (SEQ ID NO:1) for CDR-H1, as H2 in FIG. 1 (SEQ ID NO:2) or H2′ (SEQ ID NO:13) or H2″ (SEQ ID NO:15) or H2′″ (SEQ ID NO:16) for CDR-H2, or as H3 in FIG. 1 (SEQ ID NO:3) for CDR-H3, and comprises a light chain wherein the variable domain comprises a CDR having at least one of the sequences given as L1 in FIG. 1 (SEQ ID NO:4) for CDR-L1, as L2 in FIG. 1 (SEQ ID NO:5) for CDR-L2, or as L3 in FIG. 1 (SEQ ID NO:6) for CDR-L3.

48. The method of claim 46, wherein the antibody comprises a heavy chain wherein the variable domain comprises a CDR having at least one of the sequences given in SEQ ID NO:1 for CDR-H1, SEQ ID NO:2 or SEQ ID NO:13 or SEQ ID NO:15 or SEQ ID NO:16 for CDR-H2, or SEQ ID NO:3 for CDR-H3, and a light chain wherein the variable domain comprises a CDR having at least one of the sequences given in SEQ ID NO:4 for CDR-L1, SEQ ID NO:5 for CDR-L2, or SEQ ID NO:6 for CDR-L3.

49. The method of claim 46, wherein the antibody molecule comprises SEQ ID NO:1 for CDR-H1, SEQ ID NO: 2 or SEQ ID NO:13 or SEQ ID NO:15 or SEQ ID NO:16 for CDR-H2, SEQ ID NO:3 for CDR-H3, SEQ ID NO:4 for CDR-L1, SEQ ID NO:5 for CDR-L2 and SEQ ID NO:6 for CDR-L3.

50. The method of claim 46, wherein the antibody is a humanized antibody.

51. The method of claim 50, wherein the humanized antibody comprises a variable domain comprising human acceptor framework regions and non-human donor CDRs.

52. The method of claim 51, wherein the human acceptor framework regions of the variable domain of the heavy chain of the antibody are based on a human sub-group I consensus sequence and comprise non-human donor residues at positions 1, 28, 48, 71 and 93.

53. The method of claim 52, wherein the humanized antibody further comprises non-human donor residues at positions 67 and 69.

54. The method of claim 52, wherein the humanized antibody comprises a variable domain of the light chain comprising a human acceptor framework region based on a human sub-group I consensus sequence and further comprising non-human donor residues at positions 2, 4, 37, 38, 45 and 60.

55. The method of claim 52, wherein the humanized antibody further comprises a non-human donor residue at position 3.

56. The method of claim 52, wherein the humanized antibody comprises a light chain variable region 5/44-gL1 (SEQ ID NO:19) and a heavy chain variable region 5/44-gH7 (SEQ ID NO:27).

57. The method of claim 52, wherein the humanized antibody comprises a light chain having the sequence as set forth in SEQ ID NO: 28.

58. The method of claim 52, wherein the humanized antibody comprises a heavy chain having the sequence as set forth in SEQ ID NO:30.

59. The method of claim 52, wherein the humanized antibody comprises a light chain having the sequence as set forth in SEQ ID NO: 28 and a heavy chain having the sequence as set forth in SEQ ID NO: 30.

60. The method of claim 52, wherein the humanized antibody is a variant antibody obtained by an affinity maturation protocol and has increased specificity for human CD22.

61. The method of claim 60, wherein the anti-CD22 antibody is a chimeric antibody comprising the sequences of the light and heavy chain variable domains of the monoclonal antibody set forth in SEQ ID NO:7 and SEQ ID NO:8 respectively.

62. The method of claim 60, wherein the anti-CD22 antibody comprises a hybrid CDR comprising a truncated donor CDR sequence wherein the missing portion of the donor CDR is replaced by a different sequence and forms a functional CDR.

63. The method of claim 46, wherein the calicheamicin derivative is a gamma calicheamicin or a N-acetyl gamma calicheamicin derivative.

64. The method of claim 46, wherein the calicheamicin derivative is functionalized with 3-mercapto-3-methyl butanoyl hydrazide.

65. The method of claim 46, wherein the hydrolyzable linker is a bifunctional linker that is capable of releasing the calicheamicin derivative from the conjugate after binding and entry into target cells.

66. The method of claim 65, wherein the bifunctional linker is 4-(4-acetylphenoxy) butanoic acid (AcBut).

67. The method of claim 46, additionally comprising administering to the subject an anti-cytokine agent.

68. A method of treating an autoimmune disease in a subject comprising administering a therapeutically effective amount of a stable lyophilized composition of a monomeric cytotoxic drug/B cell depleting agent conjugate, said conjugate being prepared by a method comprising:
dissolving the monomeric cytotoxic drug/B cell depleting agent conjugate to a final concentration of 0.5 to 2 mg/mL in a solution comprising a cryoprotectant at a concentration of 1.5%-5% by weight, a polymeric bulking agent at a concentration of 0.5-1.5% by weight, electrolytes at a concentration of 0.01 M to 0.1 M, a solubility facilitating agent at a concentration of 0.005-0.05% by weight, buffering agent at a concentration of 5-50 mM such that the final pH of the solution is 7.8-8.2, and water; dispensing the above solution into vials at a temperature of +5° C. to +10° C.;
freezing the solution at a freezing temperature of −35° C. to −50° C.;
subjecting the frozen solution to an initial freeze drying step at a primary drying pressure of 20 to 80 microns at a shelf-temperature at −10° C. to −40° C. for 24 to 78 hours; and
subjecting the freeze-dried product of step (d) to a secondary drying step at a drying pressure of 20 to 80 microns at a shelf temperature of +10° C. to +35° C. for 15 to 30 hours.

69. The method of claim 68, additionally comprising administering to the subject an anti-cytokine agent.

70. A method for treating an autoimmune disease in a subject comprising:
administering to the subject a therapeutically effective amount of a cytotoxic drug/B cell depleting agent conjugate, wherein said B cell depleting agent is an antibody.

71. The method of claim 70, wherein the antibody is selected from the group consisting of anti-CD19, anti-CD20, and anti-CD22 antibodies.

72. The method of claim 70, wherein the antibody is a humanized antibody.

73. The method of claim 72, wherein the humanized antibody is a humanized anti-CD22 antibody.

74. The method of claim 73, wherein the humanized anti-CD22 antibody is a CDR-grafted antibody, and comprises a light chain variable region 5/44-gL1 (SEQ ID NO:19), and a heavy chain variable region 5/44-gH7 (SEQ ID NO:27).

75. The method of claim 73, wherein the humanized anti-CD22 antibody is a CDR-grafted antibody comprising a light chain having a sequence set forth in SEQ ID NO: 28.

76. The method of claim 73, wherein the humanized anti-CD22 antibody is a CDR-grafted antibody comprising a heavy chain having a sequence set forth in SEQ ID NO:30.

77. The method of claim 73, wherein the humanized anti-CD22 antibody is a CDR-grafted antibody comprising a light chain having a sequence set forth in SEQ ID NO: 28 and a heavy chain having a sequence set forth in SEQ ID NO: 30.

78. The method of claim 73, wherein the humanized anti-CD22 antibody is a CDR-grafted antibody that is a variant antibody obtained by an affinity maturation protocol and has increased specificity for human CD22.

79. A method for treating an autoimmune disease in a subject comprising:
administering to the subject a therapeutically effective amount of a B cell depleting agent, wherein the B cell depleting agent is a humanized antibody against CD22, CD19 or CD20.

80. The method of claim 79, wherein the humanized antibody is a humanized anti-CD22 antibody.

81. The method of claim 80, wherein the humanized anti-CD22 antibody is a CDR-grafted antibody, and comprises a light chain variable region 5/44-gL1 (SEQ ID NO:19), and a heavy chain variable region 5/44-gH7 (SEQ ID NO:27).

82. The method of claim 80, wherein the humanized anti-CD22 antibody is a CDR-grafted antibody comprising a light chain having a sequence set forth in SEQ ID NO: 28.

83. The method of claim 80, wherein the humanized anti-CD22 antibody is a CDR-grafted antibody comprising a heavy chain having a sequence set forth in SEQ ID NO:30.

84. The method of claim 80, wherein the humanized anti-CD22 antibody is a CDR-grafted antibody comprising a light chain having a sequence set forth in SEQ ID NO: 28 and a heavy chain having a sequence set forth in SEQ ID NO: 30.

85. The method of claim 80, wherein the humanized anti-CD22 antibody is a CDR-grafted antibody that is a variant antibody obtained by an affinity maturation protocol and has increased specificity for human CD22.

86. The method of claim 1, wherein the B cell depleting agent is used in the preparation of a medicament for the treatment of autoimmune disease in a subject.

87. A composition comprising:
(a) a cytotoxic drug/B cell depleting agent conjugate comprising at least one cytotoxic drug conjugated to at least one B cell depleting agent; and
(b) at least one anti-cytokine agent.

88. The composition of claim 87, wherein the at least one B cell depleting agent conjugated to the cytotoxic drug is selected from a group consisting of a monoclonal antibody, a chimeric antibody, a human antibody, a humanized antibody, a human antibody produced in a transgenic animal, a single chain antibody, a Fab fragment and a F(ab)2 fragment.

89. The composition of claim 87, wherein the antibody conjugated to the cytotoxic drug is selected from a group consisting of anti-CD19, anti-CD20, and anti-CD22 antibodies.

90. The composition of claim 87, wherein the cytotoxic drug/B cell depleting agent conjugate is prepared by a method comprising:
(4) adding the cytotoxic drug derivative to the bioactive agent wherein the cytotoxic drug derivative is 4.5-11% by weight of the bioactive agent;
(5) incubating the cytotoxic drug derivative and a bioactive agent in a non-nucleophilic, protein-compatible, buffered solution having a pH in the range from about 7 to 9 to produce a monomeric cytotoxic drug/B cell depleting agent conjugate, wherein the solution further comprises (a) an organic cosolvent, and (b) an additive comprising at least one C₆-C₁₈ carboxylic acid or its salt, and wherein the incubation is conducted at a temperature ranging from about 30° C. to about 35° C. for a period of time ranging from about 15 minutes to 24 hours; and
(6) subjecting the conjugate produced in step (2) to a chromatographic separation process to separate monomeric cytotoxic drug derivative/bioactive agent conjugates with a loading in the range of 4-10% by weight cytotoxic drug and with low conjugated fraction (LCF) below 10 percent from unconjugated bioactive agent, cytotoxic drug derivative, and aggregated conjugates.


SEQUENCE LISTING (51 sequences)

SEQ ID NO: 1 - protein, 5 aa, Mus musculus
NYWIH

SEQ ID NO: 2 - protein, 17 aa, Mus musculus
GINPGNNYTTYKRNLKG

SEQ ID NO: 3 - protein, 12 aa, Mus musculus
EGYGNYGAWFAY

SEQ ID NO: 4 - protein, 16 aa, Mus musculus
RSSQSLANSYGNTFLS

SEQ ID NO: 5 - protein, 7 aa, Mus musculus
GISNRFS

SEQ ID NO: 6 - protein, 9 aa, Mus musculus
LQGTHQPYT

SEQ ID NO: 7 - protein, 113 aa, Mus musculus
DVVVTQTPLSLPVSFGDQVSISCRSSQSLANSYGNTFLSWYLHKPGQSPQLLIYGISNRFSGVPDRFTGSGSGTDFTLKISTIKPEDLGMYYCLQGTHQPYTFGGGTKLEIKR

SEQ ID NO: 8 - protein, 121 aa, Mus musculus
EVQLQQSGTVLARPGASVKMSCKASGYRFTNYWIHWVKQRPGQGLEWIGGINPGNNYTTYKRNLKGKATLTAVTSASTAYMDLSSLTSEDSAVYYCTREGYGNYGAWFAYWGQGTLVTVSS

SEQ ID NO: 9 - protein, 13 aa, Artificial Sequence
  Description of Artificial Sequence Synthetic peptide
GNNYTTYKRNLKG

SEQ ID NO: 10 - protein, 13 aa, Artificial Sequence
  Description of Artificial Sequence Synthetic peptide
GNQYTTYKRNLKG

SEQ ID NO: 11 - protein, 13 aa, Artificial Sequence
  Description of Artificial Sequence Synthetic peptide
GNNYATYKRNLKG

SEQ ID NO: 12 - protein, 13 aa, Artificial Sequence
  Description of Artificial Sequence Synthetic peptide
GNNYVTYKRNLKG

SEQ ID NO: 13 - protein, 19 aa, Artificial Sequence
  Description of Artificial Sequence Synthetic peptide
GINPGLYNNYATYKRNLKG

SEQ ID NO: 14 - protein, 13 aa, Artificial Sequence
  Description of Artificial Sequence Synthetic peptide
GNNYTTYRRNLKG

SEQ ID NO: 15 - protein, 17 aa, Artificial Sequence
  Description of Artificial Sequence Synthetic peptide
GINPGNNYTTYRRNLKG

SEQ ID NO: 16 - protein, 17 aa, Artificial Sequence
  Description of Artificial Sequence Synthetic peptide
GINPGNNYATYRRNLKG

SEQ ID NO: 17 - protein, 70 aa, Homo sapiens
DIQMTQSPSSLSASVGDRVTITCWYQQKPGKAPKLLIYGVPSRFSGSGSGTDFTLTISSLQPEDFATYYC

SEQ ID NO: 18 - protein, 11 aa, Homo sapiens
FGQGTKVEIKR

SEQ ID NO: 19 - protein, 113 aa, Artificial Sequence
  Description of Artificial Sequence Synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 20 - protein, 113 aa, Artificial Sequence
  Description of Artificial Sequence Synthetic construct
DVVVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 21 - protein, 80 aa, Homo sapiens
QVQLVQSGAEVKKPGASVKVSCKASGYTFTWVRQAPGGGLEWMGKFQGRVTMTRDTSTSTVYMELSSLRSEATAVYYCAR

SEQ ID NO: 22 - protein, 11 aa, Homo sapiens
WGQGTLVTVSS

SEQ ID NO: 23 - protein, 121 aa, Artificial Sequence
  Description of Artificial Sequence Synthetic construct
EVQLVQSGAEVKKPGASVKVSCKASGYRFTNYWIHWVRQAPGQGLEWIGGINPGNQYTTYKRNLKGRATLTADTSTSTVYMELSSLRSEDTAVYYCTREGYGNYGAWFAYWGQGTLVTVSS

SEQ ID NO: 24 - protein, 121 aa, Artificial Sequence
  Description of Artificial Sequence Synthetic construct
EVQLVQSGAEVKKPGASVKVSCKASGYRFTNYWIHWVRQAPGQGLEWIGGINPGNNYATYRRNLKGRATLTADTSTSTVYMELSSLRSEDTAVYYCTREGYGNYGAWFAYWGQGTLVTVSS

SEQ ID NO: 25 - protein, 121 aa, Artificial Sequence
  Description of Artificial Sequence Synthetic construct
EVQLVQSGAEVKKPGASVKVSCKASGYRFTNYWIHWVRQAPGQGLEWIGGINPGNNYATYRRNLKGRVTMTADTSTSTVYMELSSLRSEDTAVYYCTREGYGNYGAWFAYWGQGTLVTVSS

SEQ ID NO: 26 - protein, 121 aa, Artificial Sequence
  Description of Artificial Sequence Synthetic construct
EVQLVQSGAEVKKPGASVKVSCKASGYRFTNYWIHWVRQAPGQGLEWIGGINPGNNYATYRRKFQGRATLTADTSTSTVYMELSSLRSEDTAVYYCTREGYGNYGAWFAYWGQGTLVTVSS

SEQ ID NO: 27 - protein, 121 aa, Artificial Sequence
  Description of Artificial Sequence Synthetic construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 28 - protein, 239 aa, Artificial Sequence
  Description of Artificial Sequence Synthetic construct
MKLPVRLLVLLLFWIPASRGDVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 781 bp, Artificial Sequence
  Description of Artificial Sequence Synthetic construct
ttcgaagccgccaccatgaagttgcctgttaggctgttggtgcttctgttgttctggattcctgcttcccggggtgacgttcaagtgacccagagcccatccagcctgagcgcatctgtaggagaccgggtcaccatcacttgtagatccagtcagagtcttgcaaacagttatgggaacacctttttgtcttggtatctgcacaaaccaggtaaagccccacaattgctcatctacggaatctctaacagatttagtggtgtaccagacaggttcagcggttccggaagtggtactgatttcaccctcacgatctcgtctctccagccagaagatttcgccacttattactgtttacaaggtacacatcagccgtacacattcggtcagggtactaaagtagaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgggaattc

SEQ ID NO: 30 - protein, 467 aa, Artificial Sequence
  Description of Artificial Sequence Synthetic construct
MDFGFSLVFLALILKGVQCEVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 2160 bp, Artificial Sequence
  Description of Artificial Sequence Synthetic construct
aagcttgccgccaccatggacttcggattctctctcgtgttcctggcactcattctcaagggagtgcagtgtgaggtgcaattggtccagtcaggagcagaggttaagaagcctggtgcttccgtcaaagtttcgtgtaaggctagcggctacaggttcacaaattattggattcattgggtcaggcaggctccgggacaaggcctggaatggatcggtggcattaatcccgggaataactacgctacatataggagaaaattccagggcagagttacgatgaccgcggacacctccacaagcactgtctacatggagctgtcatctctgagatccgaggacaccgcagtgtactattgtactagagaaggctacggtaattacggagcctggttcgcctactggggccagggtaccctagtcacagtctcctcagcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctggctcgaattc

SEQ ID NO: 32 - DNA, 94 bp, Artificial Sequence
  Description of Artificial Sequence Synthetic oligonucleotide
agtgtgaggtgcaattggtccagtcaggagcagaggttaagaagcctggtgcttccgtcaaagtttcgtgtaaggctagcggctacaggttcac

SEQ ID NO: 33 - DNA, 96 bp, Artificial Sequence
  Description of Artificial Sequence Synthetic oligonucleotide
gtggcattaatcccgggaatcagtacactacatataaaagaaatctaaagggcagagcaacgctgaccgcggacacctccacaagcactgtctaca

SEQ ID NO: 34 - DNA, 95 bp, Artificial Sequence
  Description of Artificial Sequence Synthetic oligonucleotide
agagaaggctacggtaattacggagcctggttcgcctactggggccagggtaccctagtcacagtctcctcagcttctacaaagggcccaagaaa

SEQ ID NO: 35 - DNA, 94 bp, Artificial Sequence
  Description of Artificial Sequence Synthetic oligonucleotide
ggaccaattgcacctcacactgcactcccttgagaatgagtgccaggaacacgagagagaatccgaagtccatggtggcggcaagcttttattc

SEQ ID NO: 36 - DNA, 97 bp, Artificial Sequence
  Description of Artificial Sequence Synthetic oligonucleotide
gattcccgggattaatgccaccgatccattccaggccttgtcccggagcctgcctgacccaatgaatccaataatttgtgaacctgtagccgctagc

SEQ ID NO: 37 - DNA, 93 bp, Artificial Sequence
  Description of Artificial Sequence Synthetic oligonucleotide
cgtaattaccgtagccttctctagtacaatagtacactgcggtgtcctcggatctcagagatgacagctccatgtagacagtgcttgtggagg

SEQ ID NO: 38 - DNA, 21 bp, Artificial Sequence
  Description of Artificial Sequence Synthetic oligonucleotide
gaataaaagcttgccgccacc

SEQ ID NO: 39 - DNA, 22 bp, Artificial Sequence
  Description of Artificial Sequence Synthetic oligonucleotide
tttcttgggccctttgtagaag

SEQ ID NO: 40 - DNA, 87 bp, Artificial Sequence
  Description of Artificial Sequence Synthetic oligonucleotide
gcttcccggggtgacgttcaagtgacccagagcccatccagcctgagcgcatctgtaggagaccgggtcaccatcacttgtagatcc

SEQ ID NO: 41 - DNA, 90 bp, Artificial Sequence
  Description of Artificial Sequence Synthetic oligonucleotide
tatctgcacaaaccaggtaaagccccacaattgctcatctacggaatctctaacagatttagtggtgtaccagacaggttcagcggttcc

SEQ ID NO: 42 - DNA, 91 bp, Artificial Sequence
  Description of Artificial Sequence Synthetic oligonucleotide
agatttcgccacttattactgtttacaaggtacacatcagccgtacacattcggtcagggtactaaagtagaaatcaaacgtacggcgtgc

SEQ ID NO: 43 - DNA, 88 bp, Artificial Sequence
  Description of Artificial Sequence Synthetic oligonucleotide
gaacgtcaccccgggaagcaggaatccagaacaacagaagcaccaacagcctaacaggcaacttcatggtggcggcttcgaatcatcc

SEQ ID NO: 44 - DNA, 88 bp, Artificial Sequence
  Description of Artificial Sequence Synthetic oligonucleotide
ctttacctggtttgtgcagataccaagacaaaaaggtgttcccataactgtttgcaagactctgactggatctacaagtgatggtgac

SEQ ID NO: 45 - DNA, 90 bp, Artificial Sequence
  Description of Artificial Sequence Synthetic oligonucleotide
aacagtaataagtggcgaaatcttctggctggagagacgagatcgtgagggtgaaatcagtaccacttccggaaccgctgaacctgtctg

SEQ ID NO: 46 - DNA, 20 bp, Artificial Sequence
  Description of Artificial Sequence Synthetic oligonucleotide
ggatgattcgaagccgccac

SEQ ID NO: 47 - DNA, 21 bp, Artificial Sequence
  Description of Artificial Sequence Synthetic oligonucleotide
gcacgccgtacgtttgatttc

SEQ ID NO: 48 - DNA, 339 bp, Mus musculus
gatgttgtggtgactcaaactccactctccctgcctgtcagctttggagatcaagtttctatctcttgcaggtctagtcagagtcttgcaaacagttatgggaacacctttttgtcttggtacctgcacaagcctggccagtctccacagctcctcatctatgggatttccaacagattttctggggtgccagacaggttcactggcagtggttcagggacagatttcacactcaagatcagcacaataaagcctgaggacttgggaatgtattactgcttacaaggtacacatcagccgtacacgttcggaggggggaccaagctggaaataaaacgt

SEQ ID NO: 49 - DNA, 363 bp, Mus musculus
gaggtccaactgcagcagtctgggactgtactggcaaggcctggggcttccgtgaagatgtcctgcaaggcttctggctacaggtttaccaactactggattcactgggtaaaacagaggcctgggcagggtctagaatggattggtggtattaatcctggaaataattatactacgtataagaggaacttgaagggcaaggccacactgactgcagtcacatccgccagcactgcctacatggacctcagcagcctgacaagtgaggactctgcggtctattactgtacaagagagggctatggtaactacggggcctggtttgcttactggggccaggggactctggtcaccgtctcctca

SEQ ID NO: 50 - DNA, 9 bp, Artificial Sequence
  Description of Artificial Sequence Synthetic oligonucleotide
gccgccacc

SEQ ID NO: 51 - DNA, 101 bp, Artificial Sequence
  Description of Artificial Sequence Synthetic primer
gcgcgcaagcttgccgccaccatggacttcggattctctctcgtgttcctggcactcattctcaagggagtgcagtgtgaggtgcagctcgtcgagtctgg